(19) United States
(12) Patent Application Publication
(10) Pub. No.: US 2025/0325705 A1
(43) Pub. Date: Oct. 23, 2025

(54) GENE THERAPY

(71) Applicants: Ospedale San Raffaele S.r.l., Milan (IT); Fondazione Telethon ETS, Rome (IT)

(72) Inventors: Anna Christina Kajaste-Rudnitski, Milan (IT); Francesco Piras, Milan (IT)

(21) Appl. No.: 18/763,059

(22) Filed: Jul. 3, 2024

Related U.S. Application Data
(63) Continuation of application No. 16/302,120, filed on May 6, 2019, now Pat. No. 12,059,478.

(30) Foreign Application Priority Data
May 20, 2016 (EP) 1608944.3

(51) Int. Cl. A61K 48/00; A61P 7/06; A61P 43/00; C07K 14/47; C12N 15/86

(52) U.S. Cl. CPC A61K 48/0058; A61K 48/0008; A61K 48/0066; A61K 48/0091; A61P 7/06; A61P 43/00; C07K 14/4746; C12N 15/86; C12N 2750/14132; C12N 2750/14141

(57) ABSTRACT

An inhibitor of p53 activation for use in haematopoietic stem and/or progenitor cell gene therapy, preferably wherein the inhibitor is an inhibitor of p53 phosphorylation, more preferably an inhibitor of p53 Serine 15 phosphorylation.

### CROSS-REFERENCE TO RELATED APPLICATIONS

This application is a continuation of U.S. patent application Ser. No. 16/302,120 (§ 371 (e) date May 6, 2019), which is a U.S. national stage filing of International Patent Application No. PCT/EP2017/062197, filed on May 19, 2017, which claims priority under 35 USC § 119 from Application No. 1608944.3, filed on May 20, 2016, in the United Kingdom.

### INCORPORATION BY REFERENCE OF MATERIAL SUBMITTED ELECTRONICALLY

Incorporated by reference in its entirety is a computer-readable nucleotide/amino acid sequence listing identified as follows: 46,909 byte extensible Markup Language (.xml) file named “53682A_SeqListing.xml”; created on Jul. 3, 2024.

### FIELD OF THE INVENTION

The present invention relates to the genetic modification of haematopoietic stem and progenitor cells. In particular, the invention relates to the use of agents for increasing the survival and engraftment of haematopoietic stem and progenitor cells transduced with viral vectors.

### BACKGROUND TO THE INVENTION

The haematopoietic system is a complex hierarchy of cells of different mature cell lineages. These include cells of the immune system that offer protection from pathogens, cells that carry oxygen through the body and cells involved in wound healing. All these mature cells are derived from a pool of haematopoietic stem cells (HSCs) that are capable of self-renewal and differentiation into any blood cell lineage. HSCs have the ability to replenish the entire haematopoietic system.

Haematopoietic cell transplantation (HCT) is a curative therapy for several inherited and acquired disorders. However, allogeneic HCT is limited by the poor availability of matched donors, the mortality associated with the allogeneic procedure which is mostly related to graft-versus-host disease (GvHD), and infectious complications provoked by the profound and long-lasting state of immune dysfunction.

Gene therapy approaches based on the transplantation of genetically modified autologous HSCs offer potentially improved safety and efficacy over allogeneic HCT. They are particularly relevant for patients lacking a matched donor.

The concept of stem cell gene therapy is based on the genetic modification of a relatively small number of stem cells. These persist long-term in the body by undergoing self-renewal, and generate large numbers of genetically “corrected” progeny. This can ensure a continuous supply of corrected cells for the rest of the patient's lifetime. HSCs are particularly attractive targets for gene therapy since their genetic modification will be passed to all blood cell lineages as they differentiate. Furthermore, HSCs can be easily and safely obtained, for example from bone marrow, mobilised peripheral blood and umbilical cord blood.

Efficient long-term gene modification of HSCs and their progeny benefits from technology which permits stable integration of the corrective DNA into the genome, without affecting HSC function. Accordingly, the use of integrating recombinant viral systems such as γ-retroviruses, lentiviruses and spumaviruses has dominated this field (Chang, A. H. et al. (2007) Mol. Ther. 15:445-56). Therapeutic benefits have already been achieved in γ-retrovirus-based clinical trials for Adenosine Deaminase Severe Combined Immunodeficiency (ADA-SCID; Aiuti, A. et al. (2009) N. Engl. J. Med. 360:447-58), X-linked Severe Combined Immunodeficiency (SCID-X1; Hacein-Bey-Abina, S. et al. (2010) N. Engl. J. Med. 363:355-64) and Wiskott-Aldrich syndrome (WAS; Boztug, K. et al. (2010) N. Engl. J. Med. 363:1918-27). In addition, lentiviruses have been employed as delivery vehicles in the treatment of X-linked adrenoleukodystrophy (ALD; Cartier, N. et al. (2009) Science 326: 818-23) and beta-thalassemia (Cartier, N. et al. (2010) Bull. Acad. Natl. Med. 194:255-264; discussion 264-258), and recently for metachromatic leukodystrophy (MLD; Biffi, A. et al. (2013) Science 341:1233158) and WAS (Aiuti, A. et al. (2013) Science 341:1233151).

In addition to the use of retro- and lentiviral-based vectors, vectors derived from other viruses, such as adenoviruses and adeno-associated viruses (AAV), may also be utilised for the modification of haematopoietic stem and progenitor cells.

Although substantial progress has been made in this area, difficulties remain with the methods employed for the genetic modification of haematopoietic stem and progenitor cells. In particular, the multiple hits of high vector doses required and prolonged ex vivo transduction times associated with existing methods give rise to problems with survival of the transduced haematopoietic stem and progenitor cells during culture and potentially impact their biological properties. Furthermore, improvements in the engraftment of transduced cells will greatly benefit clinical applications.

### SUMMARY OF THE INVENTION

The inventors have surprisingly found that, instead of triggering canonical innate immune pathways, transduction with lentiviral vectors (LVs) triggers ataxia telangiectasia mutated (ATM)-dependent DNA damage responses in human haematopoietic stem and progenitor cells. The inventors observed that this induction requires synthesis and nuclear import of the vector DNA, but is independent of genomic integration. Similarly, non-integrating adeno-associated viral (AAV) DNA was observed to induce p53 signalling, while gamma-retroviral transduction was found to trigger type I IFN responses through cytosolic RNA sensing.

In addition, the inventors found that LV-mediated signalling led to a delay in haematopoietic stem and progenitor cell proliferation, G0 arrest and slightly increased apoptosis in culture. These acute responses led to reduced engraftment of transduced cells in vivo at limiting cell doses, although no long-term consequences or competitive disadvantage were detected.

Following from these findings, the inventors demonstrated that inhibition of ATM prevented p53 activation and partially rescued in vitro apoptosis as well as in vivo engraftment of haematopoietic stem and progenitor cells.

While not wishing to be bound by theory, the inventors' findings suggest that the inhibition of p53 activation, for example by inhibition of phosphorylation of p53 (e.g. at Serine 15), in particular by inhibiting kinases that catalyse that phosphorylation (e.g. ATM kinase, and ataxia telangiectasia and Rad3-related protein (ATR)) will improve methods for haematopoietic stem and progenitor cell-based gene therapy.

Accordingly, in one aspect the invention provides an inhibitor of p53 activation for use in haematopoietic stem and/or progenitor cell gene therapy.

In one embodiment, the inhibitor is an inhibitor of p53 phosphorylation. In another embodiment, the inhibitor is an inhibitor of p53 Serine 15 phosphorylation.

The haematopoietic stem and/or progenitor cell gene therapy may be, for example, treatment of a disease selected from the group consisting of mucopolysaccharidosis type I (MPS-1), chronic granulomatous disorder (CGD), Fanconi anaemia (FA), sickle cell disease, Pyruvate kinase deficiency (PKD), Leukocyte adhesion deficiency (LAD), metachromatic leukodystrophy (MLD), globoid cell leukodystrophy (GLD), GM₂ gangliosidosis, thalassemia and cancer.

In another aspect, the invention provides an inhibitor of p53 activation for use in reducing or preventing neutropenia associated with haematopoietic stem and/or progenitor cell transplantation.

In another aspect, the invention provides an inhibitor of p53 activation for use in increasing survival and/or engraftment of haematopoietic stem and/or progenitor cells.

In another aspect, the invention provides the use of an inhibitor of p53 activation in a method of culturing an isolated population of haematopoietic stem and/or progenitor cells. In one embodiment, the inhibitor increases survival and/or engraftment of the haematopoietic stem and/or progenitor cells.

In another aspect, the invention provides the use of an inhibitor of p53 activation in a method of transducing an isolated population of haematopoietic stem and/or progenitor cells with a viral vector. In one embodiment, the inhibitor increases survival and/or engraftment of the haematopoietic stem and/or progenitor cells.

In another aspect, the invention provides the use of an inhibitor of p53 activation for increasing cell survival in an isolated population of haematopoietic stem and/or progenitor cells.

In one embodiment, the inhibitor is an inhibitor of p53 phosphorylation. In another embodiment, the inhibitor is an inhibitor of p53 Serine 15 phosphorylation.

In a preferred embodiment, the inhibitor is an ataxia telangiectasia mutated (ATM) kinase inhibitor. In another embodiment, the inhibitor is an ataxia telangiectasia and Rad3-related protein (ATR) inhibitor.

In one embodiment, the inhibitor is a p53 dominant negative peptide. In one embodiment, the inhibitor is GSE56.

In one embodiment, at least 1%, 2%, 3%, 4%, 5%, 6%, 7%, 8%, 9%, 10%, 15%, 20%, 25%, 30%, 40%, 50% or 75%, preferably at least 10%, more cells survive in culture for a period of time (e.g. about 6 or 12 hours, or 1, 2, 3, 4, 5, 6, 7 or more days, preferably about 2 days) when the cells have been exposed to the inhibitor than in its absence. Preferably, the period of time begins with transduction of the cells with a viral vector.

In one embodiment, the inhibitor substantially prevents or reduces apoptosis in the haematopoietic stem and/or progenitor cells, in particular during in vitro culture.

In one embodiment, at least 1%, 2%, 3%, 4%, 5%, 6%, 7%, 8%, 9%, 10%, 15%, 20%, 25%, 30%, 40%, 50% or 75%, preferably at least 25%, fewer cells become apoptotic following culture for a period of time (e.g. about 6 or 12 hours, or 1, 2, 3, 4, 5, 6, 7 or more days, preferably about 2 days) when the cells have been exposed to the inhibitor than in its absence. Preferably, the period of time begins with the transduction of the cells with a viral vector.

In one embodiment, at least 1%, 2%, 3%, 4%, 5%, 6%, 7%, 8%, 9%, 10%, 15%, 20%, 25%, 30%, 40%, 50% or 75%, preferably at least 10%, more transplanted haematopoietic stem and/or progenitor cells and/or their descendant cells (e.g. graft-derived cells) engraft in a host subject when the cells have been exposed to the inhibitor than in its absence.

In a preferred embodiment, the haematopoietic stem and/or progenitor cells are human haematopoietic stem and/or progenitor cells.

In one embodiment, the cells are haematopoietic stem cells. In one embodiment, the cells are haematopoietic progenitor cells. In one embodiment, the cells are short-term re-populating cells. In one embodiment, the cells are long-term re-populating cells.

In one embodiment, the haematopoietic stem and/or progenitor cells are CD34+ cells. In one embodiment, the haematopoietic stem and/or progenitor cells are CD34+CD38− cells. In one embodiment, the haematopoietic stem and/or progenitor cells are CD34+CD38+ cells.

In one embodiment, the population of haematopoietic stem and/or progenitor cells comprises, is enriched in or substantially consists of CD34+ cells. The population of cells may be further enriched for a particular sub-population of cells, for example CD34+CD38− cells.

In one embodiment, the population of haematopoietic stem and/or progenitor cells comprises, is enriched in or substantially consists of CD34+CD38− cells. In one embodiment, the population of haematopoietic stem and/or progenitor cells comprises, is enriched in or substantially consists of CD34+CD38+ cells.

In one embodiment, the haematopoietic stem and/or progenitor cells are transduced by a viral vector. For example, the survival and/or engraftment is increased in haematopoietic stem and/or progenitor cells transduced by a viral vector.

In another aspect, the invention provides a method of culturing a population of haematopoietic stem and/or progenitor cells comprising the step of contacting the population of cells with an inhibitor of p53 activation.

In another aspect, the invention provides a method of transducing a population of haematopoietic stem and/or progenitor cells with a viral vector comprising the steps:
(a) contacting the population of cells with an inhibitor of p53 activation; and
(b) transducing the population of cells with the viral vector.

In one embodiment, the inhibitor is an inhibitor of p53 phosphorylation. In another embodiment, the inhibitor is an inhibitor of p53 Serine 15 phosphorylation.

In a preferred embodiment, the inhibitor is an ataxia telangiectasia mutated (ATM) kinase inhibitor. In another embodiment, the inhibitor is an ataxia telangiectasia and Rad3-related protein (ATR) inhibitor.

In one embodiment, the inhibitor is a p53 dominant negative peptide. In one embodiment, the inhibitor is GSE56.

The haematopoietic stem and/or progenitor cells may be, for example, contacted with the inhibitor, and/or transduced with the viral vector in vitro or as part of an ex vivo procedure. Thus, in one embodiment of the method of transducing a population of haematopoietic stem and/or progenitor cells, steps (a) and (b) are carried out ex vivo or in vitro.

Preferably, the inhibition is transient (transient inhibition of p53 signalling during the ex vivo transduction improved engraftment by about 25%, giving rise to comparable levels of human CD45+ cells detected in the peripheral blood between transduced and control virus-exposed cells).

In one embodiment, the inhibitor is a transient inhibitor (e.g. has an inhibitory action lasting less than about 1, 2, 3, 4, 5, 6, 7 or 14 days), such as a reversible inhibitor. Preferably, the cells are exposed to the inhibitor for about 1-48 or 1-24 hours, preferably 1-24 hours. The cells may be, for example, exposed to the inhibitor at the same time as the viral vector or before the viral vector.

In a preferred embodiment, the haematopoietic stem and/or progenitor cells are human haematopoietic stem and/or progenitor cells.

In one embodiment, the cells are haematopoietic stem cells. In one embodiment, the cells are haematopoietic progenitor cells. In one embodiment, the cells are short-term re-populating cells. In one embodiment, the cells are long-term re-populating cells.

In one embodiment, the haematopoietic stem and/or progenitor cells are CD34+ cells. In one embodiment, the haematopoietic stem and/or progenitor cells are CD34+CD38− cells. In one embodiment, the haematopoietic stem and/or progenitor cells are CD34+CD38+ cells.

In one embodiment, the population of haematopoietic stem and/or progenitor cells comprises, is enriched in or substantially consists of CD34+ cells. The population of cells may be further enriched for a particular sub-population of cells, for example CD34+CD38-cells.

In one embodiment, the population of haematopoietic stem and/or progenitor cells comprises, is enriched in or substantially consists of CD34+CD38− cells. In one embodiment, the population of haematopoietic stem and/or progenitor cells comprises, is enriched in or substantially consists of CD34+CD38+ cells.

In one embodiment, the inhibitor is KU-55933 or a derivative thereof; KU-60019, BEZ235, wortmannin, CP-466722, Torin 2, CGK 733, KU-559403, AZD6738 or derivatives thereof; or an siRNA, shRNA, miRNA or antisense DNA/RNA. Preferably, the inhibitor is KU-55933 or a derivative thereof.

In one embodiment, the inhibitor (e.g. KU-55933 or derivative thereof) is added to the haematopoietic stem and/or progenitor cells (e.g. in an in vitro or ex vivo culture) at a concentration of about 1-50, 1-40, 1-30, 1-20 or 1-15 μM, preferably about 1-15 μM. In another embodiment, the inhibitor (e.g. KU-55933 or derivative thereof) is added to the haematopoietic stem and/or progenitor cells at a concentration of about 5-50, 5-40, 5-30, 5-20 or 5-15 μM, preferably about 5-15 μM.

In one embodiment, the inhibitor (e.g. KU-55933 or derivative thereof) is added to the haematopoietic stem and/or progenitor cells (e.g. in an in vitro or ex vivo culture) at a concentration of about 1, 2, 3, 4, 5, 6, 7, 8, 9, 10, 11, 12, 13, 14, 15, 20, 25, 30, 35, 40, 45 or 50 μM, preferably about 10 μM.

In one embodiment, the haematopoietic stem and/or progenitor cells (e.g. the population of cells in step (a) of the method of transducing a population of haematopoietic stem and/or progenitor cells of the invention) are contacted with the inhibitor about 30 minutes to about 4 hours; about 30 minutes to about 3 hours; or about 30 minutes to about 2 hours before transducing the population of cells with the viral vector. In another embodiment, the haematopoietic stem and/or progenitor cells are contacted with the inhibitor about 1-4 hours; 1-3 hours; or 1-2 hours before transducing the population of cells with the viral vector.

In one embodiment, the haematopoietic stem and/or progenitor cells (e.g. the population of cells in step (a) of the method of transducing a population of haematopoietic stem and/or progenitor cells of the invention) are contacted with the inhibitor about 30 minutes, 1 hour, 1.5 hours, 2 hours, 2.5 hours, 3 hours, 3.5 hours or 4 hours, preferably about 2 hours, before transducing the population of cells with the viral vector.

In one embodiment, the viral vector is a lentiviral or adeno-associated viral (AAV) vector. The lentiviral vector may be, for example, a vector derived from HIV-1, HIV-2, EIAV, SIV or FIV. Preferably, the lentiviral vector is a vector derived from HIV-1. The AAV vector may be, for example, a vector comprising an AAV serotype 1, 2, 3, 4, 5, 6, 7, 8, 9, 10, or 11 capsid (when the vector is in the form of a viral vector particle) and/or genome. Preferably, the AAV vector is a vector comprising an AAV serotype 6 capsid and/or genome.

In one embodiment, the population of haematopoietic stem and/or progenitor cells is obtained from mobilised peripheral blood, bone marrow or umbilical cord blood.

In one embodiment, the method of transducing a population of haematopoietic stem and/or progenitor cells includes a further step of enriching the population for haematopoietic stem and/or progenitor cells.

In another aspect, the invention provides a method of gene therapy comprising the steps:
(a) transducing a population of haematopoietic stem and/or progenitor cells according to the method of the invention; and
(b) administering the transduced cells to a subject.

The method of gene therapy may be, for example, a method of treatment of a disease selected from the group consisting of mucopolysaccharidosis type I (MPS-1), chronic granulomatous disorder (CGD), Fanconi anaemia (FA), sickle cell disease, Pyruvate kinase deficiency (PKD), Leukocyte adhesion deficiency (LAD), metachromatic leukodystrophy (MLD), globoid cell leukodystrophy (GLD), GM₂ gangliosidosis, thalassemia and cancer.

In one embodiment, the subject is a mammalian subject, preferably a human subject.

In one embodiment, the transduced cells are administered to a subject as part of an autologous stem cell transplant procedure or an allogeneic stem cell transplant procedure.

In another aspect, the invention provides a population of haematopoietic stem and/or progenitor cells prepared according to the method of the invention.

In another aspect, the invention provides a pharmaceutical composition comprising the population of haematopoietic stem and/or progenitor cells of the invention.

In another aspect, the invention provides the population of haematopoietic stem and/or progenitor cells of the invention for use in therapy.

In one embodiment, the population of haematopoietic stem and/or progenitor cells of the invention are administered to a subject as part of an autologous stem cell transplant procedure or an allogeneic stem cell transplant procedure.

In another aspect the invention provides the use of an inhibitor of p53 activation for the manufacture of a medicament for haematopoietic stem and/or progenitor cell gene therapy.

In another aspect the invention provides the use of an inhibitor of p53 activation for the manufacture of a medicament for increasing survival and/or engraftment of haematopoietic stem and/or progenitor cells.

In another aspect, the invention provides a method of increasing survival and/or engraftment of haematopoietic stem and/or progenitor cells comprising the steps:
(a) contacting a population of haematopoietic stem and/or progenitor cells with an inhibitor of p53 activation; and
(b) transducing the population of cells with a viral vector.

In another aspect, the invention provides an inhibitor of p53 activation for use in a method of transducing a population of haematopoietic stem and/or progenitor cells with a viral vector.

The inhibitor may be as disclosed herein.

In another aspect, the invention provides a method of screening for an agent capable of increasing survival and/or engraftment of haematopoietic stem and/or progenitor cells comprising the steps:
(a) contacting a population of haematopoietic stem and/or progenitor cells with a candidate agent;
(b) transducing the population of cells with a viral vector; and optionally
(c) administering the transduced cells to a subject,

Cell survival and/or engraftment may be subsequently analysed using a method as disclosed herein. Preferably, the candidate agent has been identified as an inhibitor of p53 activation, in particular p53 phosphorylation, for example Serine 15 phosphorylation (e.g. preferably an ATM kinase inhibitor or ATR inhibitor) using a kinase activity assay, for example as disclosed herein.

### DESCRIPTION OF THE DRAWINGS

FIG. 1. Lentiviral transduction induces p53 and DDR transcriptional signalling in human HSPC. (A) Scheme of the RNA-Seq experiment. After 16 hours of cytokine prestimulation on human cord-blood (CB)-derived CD34+ cells were exposed to Lab-grade (Lab LV) or purified (Purified LV) LV at a multiplicity of infection (MOI) of 100, kept in culture untreated or exposed to p24 equivalent of non-transducing Env-less (Bald LV), heat inactivated purified LV or Poly(I:C). RNA was extracted at different times post-transduction and processed. Bar chart of the pathway enrichment analysis perform on the significant (nom p-value≤0.05) differentially expressed genes over time retrieved from the comparison between (B) Bald LV and the Lab LV or (C) Inactivated purified LV and the Purified LV conditions. The length of the bar represents the significance (p-value Ranking) of that specific pathway. In addition, the brighter the colour, the more significant that term is. (D) Heat map showing the profile over time of the most significantly (nom p-value≤0.01) differentially expressed genes between Lab LV and Bald LV conditions. Genes are clustered upon the semantic distances and represented over time for each separated condition. (E) Genes differentially expressed in the Lab LV condition respect to the Bald LV are highlighted by the red star in the KEGG p53 signalling pathway. (F) Gene expression levels of different p53-induced genes (p48, PUMA, p21, PHLDA3) in CB-CD34+ cells transduced with PGK-GFP SIN LV at MOI 100 (LV) or exposed to p24 equivalent of Env-less (Bald) vector as control were measured 48 hours after the transduction. Expression levels were normalised to HPRT1 and shown respect to Bald set to value of 1. Results are the mean±SEM of four independent experiments, p-values are for One sample t-test (*p≤0.05; ** p≤0.01;**** p≤0.0001).

FIG. 2. p53 induction occurs in all CD34+ subpopulations, requires reverse-transcription but is integration independent. (A) Experimental design of the sorting experiment. After 16 hours of cytokine pre-stimulation CB-CD34+ were sorted based on the expression of the CD133 and CD38 surface marker to discriminate the fraction enriched in stem cells (CD133+CD38−) from the more committed progenitors (CD133+CD38int and CD133+CD38hi). Cells were transduced immediately after sorting with an MOI 100 of PGK-GFP SIN LV or exposed to p24 equivalent of Bald as control. p21 mRNA levels were measured in (B) the different sorted CD34+ subpopulations (n=4); (C) total CB-CD34+ (n=6) or (D) bone marrow (BM)-derived CD34+ (BM-CD34+) cells (n=4) exposed to an MOI of 100 PGK-GFP SIN LV (LV), integrase-defective LV (IDLV), or p24 equivalents of genome-less (Empty LV) or Env-less (Bald) vectors; (E) CB-CD34+ transduced in presence of the integrase inhibitor Raltegravir (Ral) or reverse-transcriptase inhibitor Lamivudin (3TC) (n=4). All data is normalised to HPRT1 and shown respect to Bald set to value of 1. Results are the mean±SEM of n independent experiments as indicated, p-values are for Kruskal-Wallis test (*p≤0.05; ** p≤0.01). (F) Western blot was performed on whole CB-CD34+ cells extracts at 24 hours post-transduction. Immunoblots were sequentially probed with phospho- and non phospho-specific antibodies against p53, phospho-specific antibody against γH2AX, nonphospho-specific antibodies against p21, and β-actin (loading controls). One representative out of two gels is shown. (G) Western blot (Left Panel) and quantifications (Right Panel) were performed on whole mPB-CD34+ cell extracts at 24 hours post-transduction. (H) p21 protein upregulation evaluated by FACS 48 hours after transduction.

FIG. 3. p53 induction requires active nuclear import. mRNA levels of p21 (A-B) and IFN-stimulated genes (IRF7, OAS1 and ISG15) (C) were measured at 48 hours post-exposure of CB-CD34+ to PGK-GFP SIN LV (LV), PGK-GFP LV lacking the central polypurine tract-deleted (ΔcPPT) or PGK-GFP SIN RV (γ-RV) at MOI 100, p24 equivalent of Env-less (Bald) vector or MOI 10000 of PGK-GFP Adeno-associated Vector (AAV6) as indicated. All data are normalised to HPRT1 and shown respect to Bald set to value of 1. Results are the mean±SEM of ≥4 independent experiments p-values are for Kruskal-Wallis test (*p≤0.05; ** p≤0.01; *** p≤0.001); ++p≤0.01 is for Wilcoxon Matched paired test.

FIG. 4. Functional consequences of LV-mediated signalling in human HSPC in vitro. (A) CB-CD34+ cells were stained with the cell proliferation dye and transduced with MOI 100 of PGK-GFP SIN LV (LV), p21 overexpressing LV (p21) or p24 equivalent of Bald. Cell proliferation was followed over time in terms of dye MFI in the different subpopulations gated as shown in FIG. 13B. Representative histograms (one out of four) of the Cell proliferation dye MFI in the different conditions over time is shown in the top panel. Dye MFI at 2 days after the transduction within each subpopulation is shown in the lower panel. Results are the mean±SEM of 4 independent experiments, p-values are for Wilcoxon Matched paired test (*p≤0.05; ** p≤0.01). (B) Cell cycle analysis was performed with Anti-Ki67 antibody and Hoechst at FACS 2 days after the transduction of CB-CD34+ cells with an MOI 100 of PGK-GFP SIN LV (LV), p21 overexpressing LV (p21) or p24 equivalent of Bald. Annexin staining for apoptotic cells was performed two days after exposure of (C) total or (D) sorted CB-CD34+ to PGK-GFP SIN LV (LV), integrase-defective LV (IDLV) at MOI 100 or p24 equivalents of genome-less (Empty LV) or Env-less (Bald) as indicated. (E) A fraction of non-transduced (UT) (Bald, Empty LV, or untreated) and transduced (PGK-GFP SIN LV MOI 100/150) CB-CD34+ cells were plated in semi-solid culture and the number of White and Red colonies were counted after 2 weeks of differentiation. (F) Impact of integrase (Ral) or reverse-transcriptase (3TC) inhibition on apoptosis was evaluated in total CB-CD43+. (G) CB-CD34+ cells were counted 2, 4 and 6 days after the transduction. Results are the mean±SEM of n≥4 independent experiments, p-values are for Kruskal-Wallis test (*P≤0.05; ** P≤0.01;*** P≤0.001) in (C, D, F) and Wilcoxon Matched paired test, *P≤0.05;) in (A and E) and Two-Way ANOVA, P=0.002, with Bonferonni's post-test ** P≤0.01 at day 6 in (G).

FIG. 5. In vivo impact of LV-mediated signalling. (A) Schematic representation in vivo experimental design. 24 hours after the transduction CB-CD34+ cells were washed and injected either separately (Empty LV (n=10), LV (n=9) or in a mixed condition composed of LV:Empty LV exposed cells in a 30:70 ratio (Mix (n=9)). Secondary recipients received 900.000-1.000.000 of CD34+ cells purified from the bone marrow of primary mice sacrificed at 12 weeks post-transplantation. Percentages of (B) total human CD45+ cells as well as (C) myeloid (CD13+) and lymphoid (CD19+ B, CD3+ T) cells within the human CD45+ cells were monitored in the peripheral blood over time and (D) in the bone marrow at the time of sacrifice. The bone marrow composition at time of sacrifice of primary recipients was evaluated. Representative FACS plot and frequencies of (E) CD34+, CD34+38+ and CD34+CD38-within the human CD45+ cells and of (F) CD45RA-90+ (HSC), CD45RA-90-(MPP) and CD45RA+90− (MLP) within the human CD34+38− cells are shown. Results are shown as mean±SEM and p-values are for Kruskal-Wallis test (*P≤0.05; ** P≤0.01, *** p≤0.0001). (G) Peripheral blood and (H) frequency of CD34+ cells within the bone marrow at time of sacrifice of the secondary recipients are shown. (I) Schematic representation of the experimental design and percentages of total human CD45+ cells monitored in the peripheral blood over time and in the bone marrow at the end of the experiments. (J) Percentages and absolute numbers of human CD34+ cells retrieved from the bone marrow of NSG mice 16 h post-transplantation. (K) Table and plot of the calculated HSC frequency obtained by LDA for untransduced (Bald) and MOI 100 transduced (LV) CB-CD34+ cells. Results are the mean±SEM of Empty LV (n=10), LV (n=9), and Mix (n=11) secondary recipients per group. HSC=Hematopoietic Stem Cells, MPP=Multi potent Progenitors, MLP=Multi Lymphoid Progenitors.

FIG. 6. LV-mediated activation of p53 signalling is ATM-dependent. Human CB-CD34+ cells were transduced with MOI of 100 of PGK-GFP SIN LV (LV), or Env-less (Bald) in presence 10 μM KU55933 and levels of p21 mRNA induction (A), of cellular apoptosis (B) and transduction in terms of GFP expression and integrated vector copy numbers (VCN) (C, one representative FACS plot out of 5 is shown) were monitored 48 hours post-exposure. Impact of ATM inhibition on AAV6-mediated induction of p21 mRNA and apoptosis are shown in (D). Results are the mean±SEM of n≥3 independent experiments, p-values are for Friedman's test (n=6, *** P≤0.001) or for Wilcoxon Matched Paired test (++ for P≤0.01). (E) Schematic representation of the experimental design to test in vivo impact of ATM inhibition is shown. After 16 hours of pre-stimulation cells were transduce with PGK-GFP SIN LV at MOI 100 (LV) or p24 equivalent of bald LV in presence of KU55933 or DMSO as a control. Twenty four hours after the transduction cells were washed and injected into sub-lethally irradiated NSG mice. (F) Percentages of total human CD45+ cells over time in the peripheral blood are shown. Results are the mean±SEM of Bald DMSO (n=8), LV DMSO (n=10), and Bald KU55933 (n=11) and LV KU5933 (n=11) NSG mice.

Inhibition of LV signalling rescues HSPC apoptosis and engraftment. (G) p21 mRNA levels measured 24 hours after the second hit of transduction and 6 hours after CPT treatment in CB-CD34+ overexpressing a dominant negative form of p53 (GSE56) or GFP as control. (H) Apoptosis was evaluated 48 hours after the second hit of transduction with (Bald, LV or AAV6 vectors) in CB-CD34+ overexpressing GSE56 or GFP as control. (I) Western Blot was performed on whole mPB-CD34+ and CB-CD34+ 24 and 48 hours after the transduction, respectively, in the presence of 10 μM KU55933. (J-L) Impact of ATM inhibition on apoptosis, CFC assay and p21 mRNA induction on mPB-CD34+ cells after transduction with two hits at MOI 100 of clinical-grade LV or p21 equivalent of Inactivated Clinical LV (Inact LV) as a control. (M) In vivo impact of ATM inhibition on total human CD45+ cells in the peripheral blood over time. Results are the mean±SEM and of n≥3 independent experiments for in vitro. * p≤0.05; ** p≤0.01; *** p≤0.001.

FIG. 7. (A) Principal Component Analysis (PCA) plot. The samples from the RNA-seq experiment are shown in the 3D plane spanned by their first three principal components. The first 3 principal components accounted for the 85% of explained variance among the data set. (B) Bar chart of the pathway enrichment analysis performed with WikiPathways database on the significant (nom p-value $0.05) differentially expressed genes over time (5883) in the untreated condition. The length of the bar represents the significance (p-value Ranking) of that specific pathway. In addition, the brighter the colour, the more significant that term is.

FIG. 8. Heat map of clustered biological terms highlighted by differentially expressed genes in the untreated sample respect to the Poly(I:C) (A) The heat map represents semantic similarity among gene ontology (GO) Biological Process (BP) terms. Rows and columns show the list of enriched GO BP terms derived from term enrichment analysis of the differentially expressed genes over time between the untreated and Poly(I:C) conditions. The colours represent the semantic distances calculated using GOSemSim Bioconductor package. Yellow-red clusters identify groups of terms sharing semantic similarity about biological processes.

FIG. 9. Heat map of clustered biological terms highlighted by differentially expressed genes in the transduced samples respect to the controls (A) The heat map represents semantic similarity among gene ontology (GO) Biological Process (BP) terms. Rows and columns show the list of enriched GO BP terms derived from term enrichment analysis of the differentially expressed genes over time between the Bald LV and Lab LV conditions. The colours represent the semantic distances calculated using GOSemSim Bioconductor package. Yellow-red clusters identify groups of terms sharing semantic similarity about biological processes. (B) The heat map represents semantic similarity among gene ontology (GO) Biological Process (BP) terms. Rows and columns show the list of enriched GO BP terms derived from term enrichment analysis of the differentially expressed genes over time between the Bald LV and Lab LV conditions. The colours represent the semantic distances calculated using GOSemSim Bioconductor package. Yellow-red clusters identify groups of terms sharing semantic similarity about biological processes.

FIG. 10. p21 induction in clinical grade LV (A) p21 mRNA levels were measured after 48 hours from the transduction of CB-CD34+ with an MOI of 100 PGK-GFP SIN LV (LV), MOI of 100 clinical grade LV (Clinical LV), or to p24 equivalent of Env-less (Bald) or inactivated Clinical LV as controls (Inactivated Clinical LV). p21 mRNA was normalised to HPRT1, results are shown respect to Bald set to value 1. Results are shown as mean±SEM of two independent experiments.

FIG. 11. Characterisation of LV-induced p53 signalling. (A) (Left Panel) p21 mRNA levels 48 hours post-transduction or (Right Panel) Vector Copy number (VCN) 3 days after transduction were measured in CB-CD34+ exposed to increasing MOI of 100 PGK-GFP SIN LV (LV) or p24 equivalent of Bald as control. P21 mRNA was normalised to HPRT1. Results are shown respect to Bald set to value 1, (mean±SEM) of three independent experiments. (B) p21 mRNA levels 48 hours post-transduction in different human and murine cell lines or primary cells exposed to PGK-GFP SIN LV or p24 equivalent of Bald as control. p21 mRNA was measured 48 hours after the transduction and normalised to HPRT1 for human cells or Hprt for Murine cells. Results are shown respect to Bald for each cell type set to value 1 (Red threshold). (C) VCN per cell were performed 14 days after exposure of sorted CB-CD34+ to PGK-GFP SIN LV (LV), at MOI 100 or p24 equivalents of Env-less (Bald) as indicated. Values are mean±SEM of 4 independent experiments. (D) Copy number per cell of total viral DNA three days after the transduction of human CB-CD34+ exposed to an MOI of 100 PGK-GFP SIN LV (LV), integrase-defective LV (IDLV), p24 equivalent genome-less (Empty LV) or Env-less (Bald). Results are shown as mean±SEM. (E) Vector copy number results of CB-CD34+ transduced with an MOI of 100 of PGK-GFP SIN LV (LV), or Env-less (Bald) as control. Transductions were performed in presence of the integrase inhibitor Raltegravir (Ral), or the Reverse transcriptase inhibitor Lamivudine (3TC). As control cells were transduced in DMSO or kept untreated (Mock). VCN was performed on the integrated LV genome 14 days after transduction, values are mean±SEM of 4 independent experiments. (F) p21 mRNA levels in CB-CD34+ cells 5 days after transduction with an MOI 100 of LV, IDLV or p24 equivalent of Bald or Empty LV as controls. (G) Western blot (WB) for p53 and p21 of CB-CD34+ transduced with an MOI of 100 of PGK-GFP SIN LV (LV), or Env-less (Bald) as control. Transductions were performed in presence of the integrase inhibitor Raltegravir (Ral), or the Reverse transcriptase inhibitor Lamivudine (3TC). As control cells were transduced in DMSO or kept untreated (Mock). WB was performed 48 hours after transduction. (H) Representative FACS plot of CB-CD34+ cells 48 hours after the transduction with PGK-BFP LV, IDLV, or Bald as control and stained with anti-p21 antibody. (I) Immunofluorescence of CB-CD34+ cells stained for γH2AX (Left Panel), 12 hours after the transduction with the indicated vector or camptothecin (CPT) exposure, and for p21 (Right Panel) 48 hours after the transduction with PGK-mCHERRY LV, IDLV, a p21 overexpressing LV (p21 OE) or Bald as a control. (J) ISG15 and p21 mRNA levels in CB-CD34+ cells exposed to 1000 U/mL of human IFNα for 24 hours and transduced for 48 hours either with an MOI 100 of a PGK-GFP LV or p24 equivalent of Bald as a control.

FIG. 12. γ-RV, AAV6 and ΔcPPT (A) Percentage of GFP positive CB-CD34+ transduced with PGK-GFP SIN LV at MOI 100 (LV), or exposed to p24 equivalent of Env-less (Bald) vector as control, cells were also transduced with an MOI 100 of a PGK-GFP SIN RV or to an MOI 10000 of PGK-GFP Adeno-associated Vector (AAV6). GFP expression was assessed at FACS 5 days after transduction for the integrating vector, and 2 days post TD for the AAV6. Results are shown as mean±SEM. (B) Vector copy number results of CB-CD34+ transduced with PGK-GFP SIN LV or PGK-GFP SIN RV (RV) at MOI 100 or exposed to p24 equivalent of Env-less (Bald) vector as control. Copy per cell of total viral DNA was assessed three days after transduction, while VCN on the integrated vectors were performed 14 days after transduction. Values are mean±SEM. (C) Comparison of the nuclear import efficiency and (D) total viral DNA three days after transduction with LV or ΔcPPT. Nuclear import efficiency is reported as the percentage of 2LTR circles on the total reverse transcribed viral DNA. The same analysis was performed in 293T cells (MOI 1) (Left Panel) and in human CBCD34+ cells (MOI 100) (Right Panel). Values are mean±SEM of 6 independent experiments. (E) Interferon stimulated genes results of human CB-CD34+ transduced with an MOI of 100 of PGK-GFP SIN RV (RV), or Env-less (Bald) as control. Transductions were performed in presence of the integrase inhibitor Raltegravir (Ral), or reverse transcriptase inhibitor azidothymidine (AZT). Interferon stimulated genes (IRF7, OAS1, ISG15) were measured 48 hours post-transduction. Expression levels were normalised to HPRT1 and shown respect to Bald set to value of 1. (F) Interferon stimulated genes (IFIT1, IRF7, OAS1, and ISG15) induction of Murine HSPC transduced with an MOI of 100 of PGK-GFP SIN LV (LV), PGK-GFP SIN RV (RV), or to p24 equivalent of Env-less (Bald). Cells were also left untreated as a control in the mock condition. 48 hours post-transduction. Expression levels were normalised to HPRT1 and shown respect to mock set to value of 1.

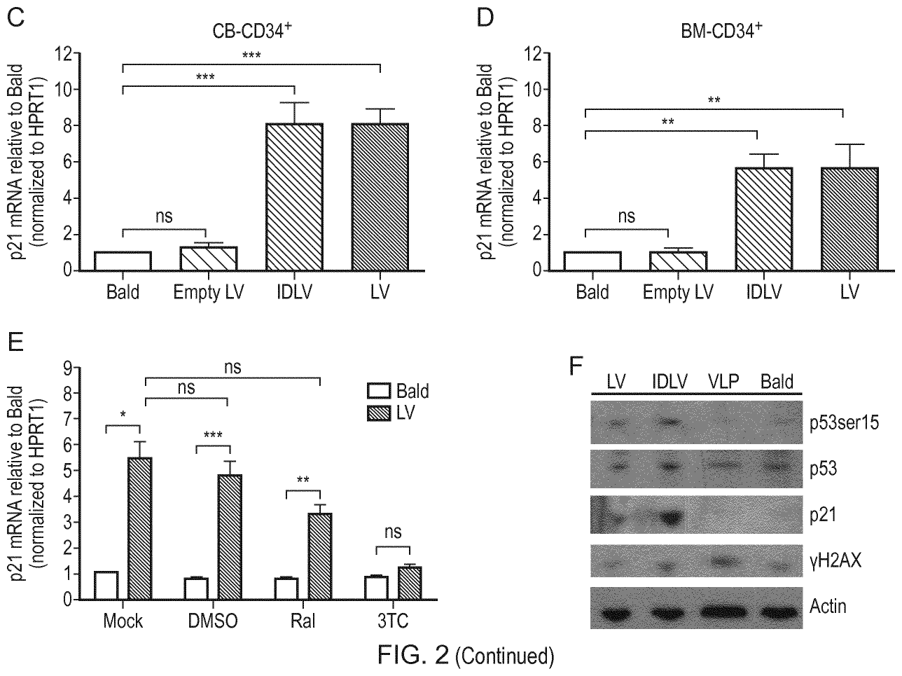

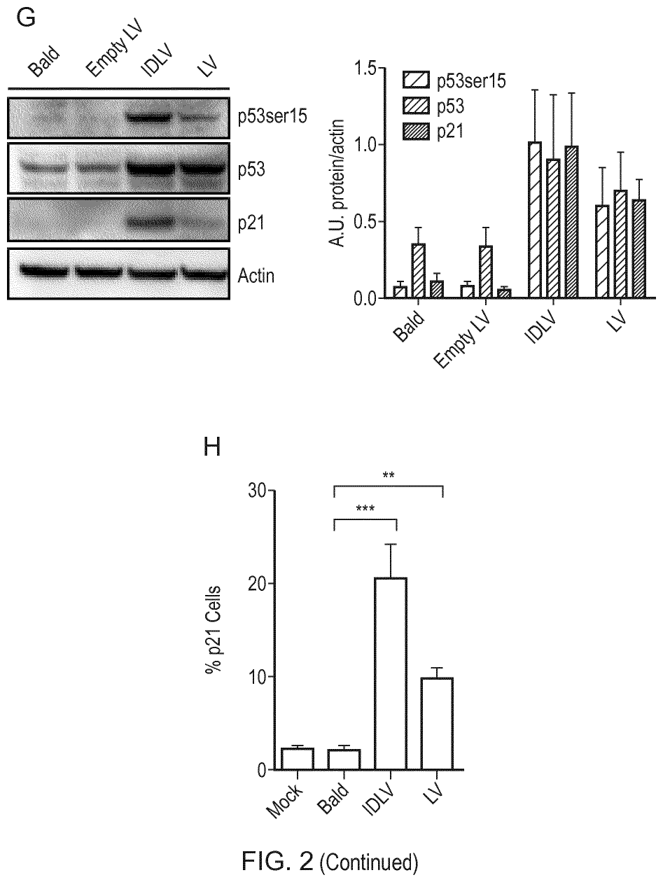

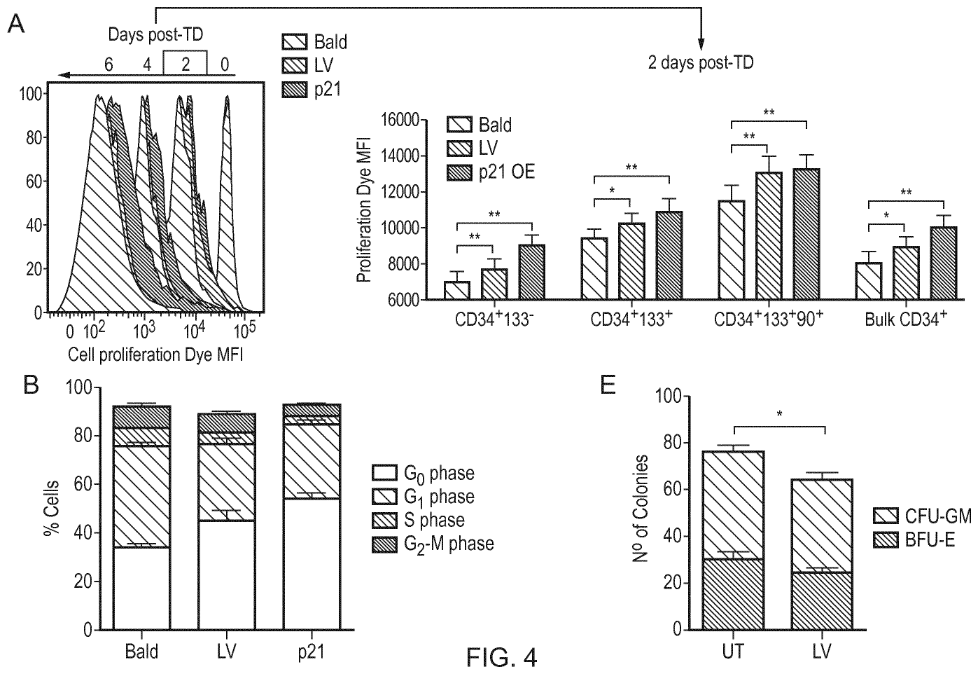

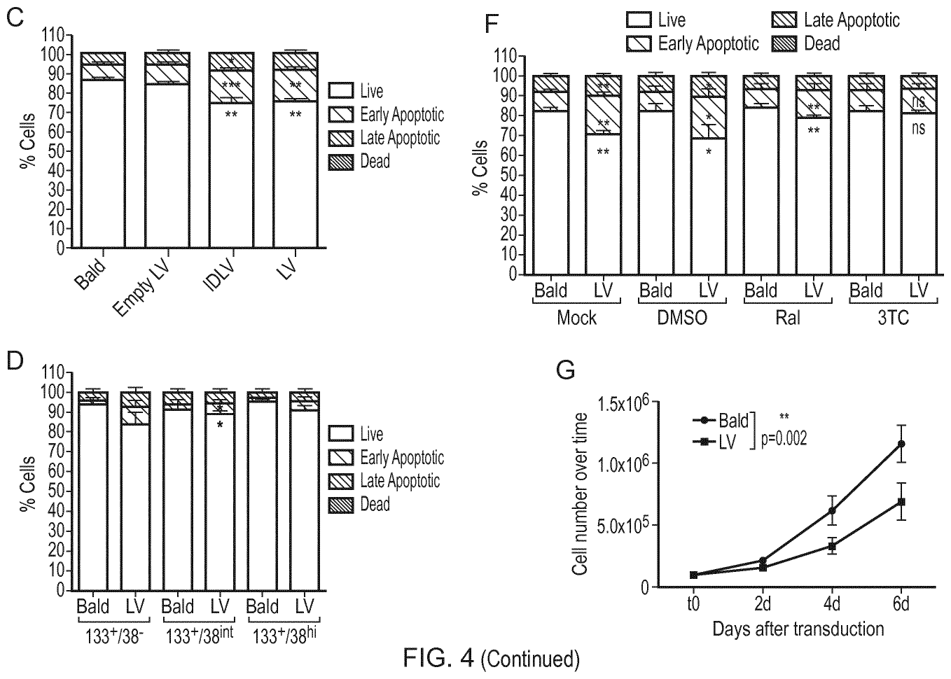

FIG. 13. In vitro impact of LV (A) p21 expression levels 48 hours after the transduction of human CB-CD34+ exposed to an MOI of 100 PGK-GFP SIN LV (LV), p21 overexpressing LV (p21) or p24 equivalent of Bald. p21 mRNA was normalised to HPRT1. Results are mean±SEM of two independent experiments shown respect to Bald set to value 1. (B) Representative gating strategy to analyse the CD34+ cells treated as in FIG. 4A. The upper panel shows the percentage of CD34, CD133 and CD90 at the moment of transduction, the lower panel reports the same markers two days after the transduction. In The left panel the bar graphs show the MFI of the cell proliferation dye at the moment of transduction or 4 and 6 days after the transduction within each subpopulation. Results are shown in mean±SEM of four independent experiments. (C) Representative histograms and (D) bar graphs of cells treated as in FIG. 4A at two days after transduction. The analyses was performed to assess the percentage of CBCD34+ cells that are contained within each gate (C1, C2, C3) for LV, p21 and Bald conditions. The cells within the C1 gate have proliferated less, while the one in the C3 have proliferated more. Results are shown in mean±SEM of four independent experiments. (E) CD34+ HSPCs subpopulation distribution at the moment of transduction or 2, 4 and 6 days after the transduction. (F) After the sorting indicated in FIG. 2A and the transduction, CBCD34+ cells were plated in semisolid, cytokine-containing CFC medium. Colonies were scored after 14 days. Mean±SEM of total colonies per 800 transduced or bald exposed cells per subpopulation (n=4). (G) Annexin V staining for apoptotic cells was performed two days after transduction of human CB-CD34+ exposed to increasing MOI of PGK-GFP SIN LV (LV).

FIG. 14. In vitro impact of clinical-grade LV and other vectors. (A-B) Annexin staining for apoptotic cells was performed two days after transduction of Human CB-CD34+ exposed to an MOI of 100 PGK-GFP SIN LV (LV), MOI of 100 clinical grade LV (Clinical LV), MOI 10000 of AAV6 and MOI 100 OF PGK-GFP SINRV (RV) or to p24 equivalent of Env-less (Bald) or inactivated Clinical LV as controls (Inactivated Clinical LV). Mean±SEM of 2 (A) or n (B) independent experiments. Statistic evaluation was performed by Kruskal-Wallis test (*p≤0.05; ** p≤0.01).

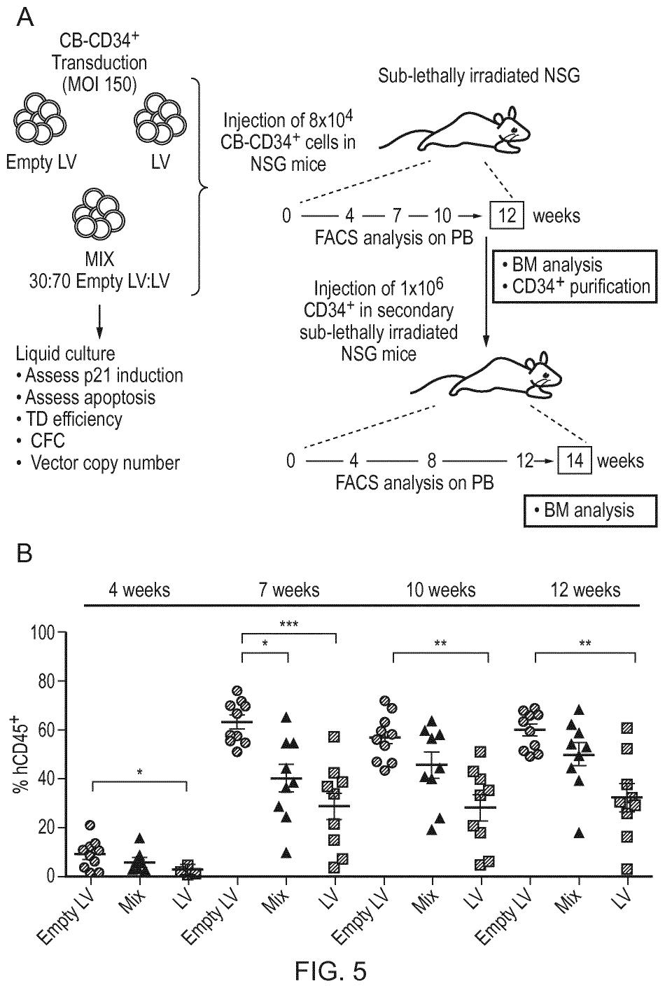

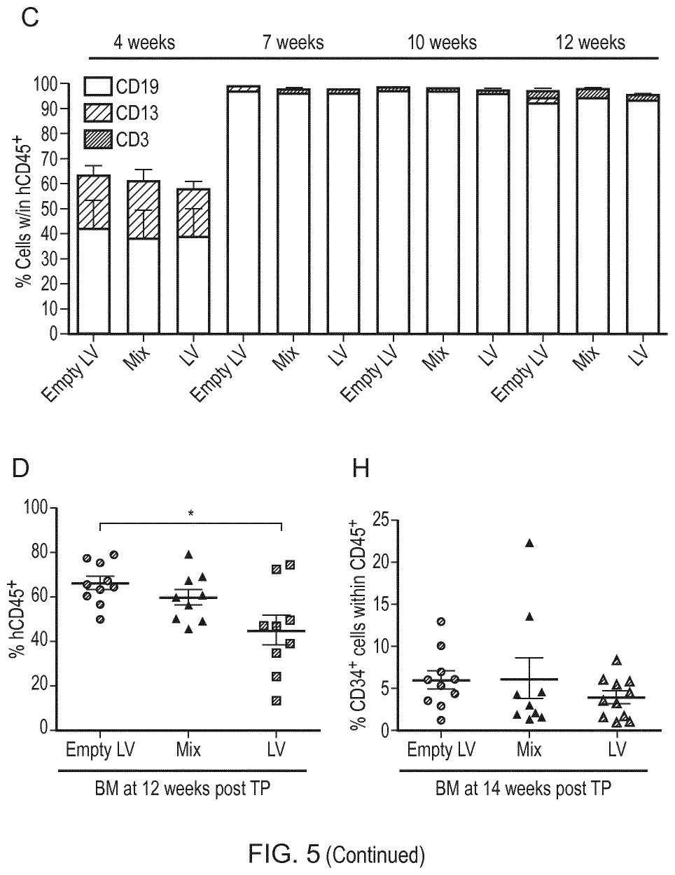

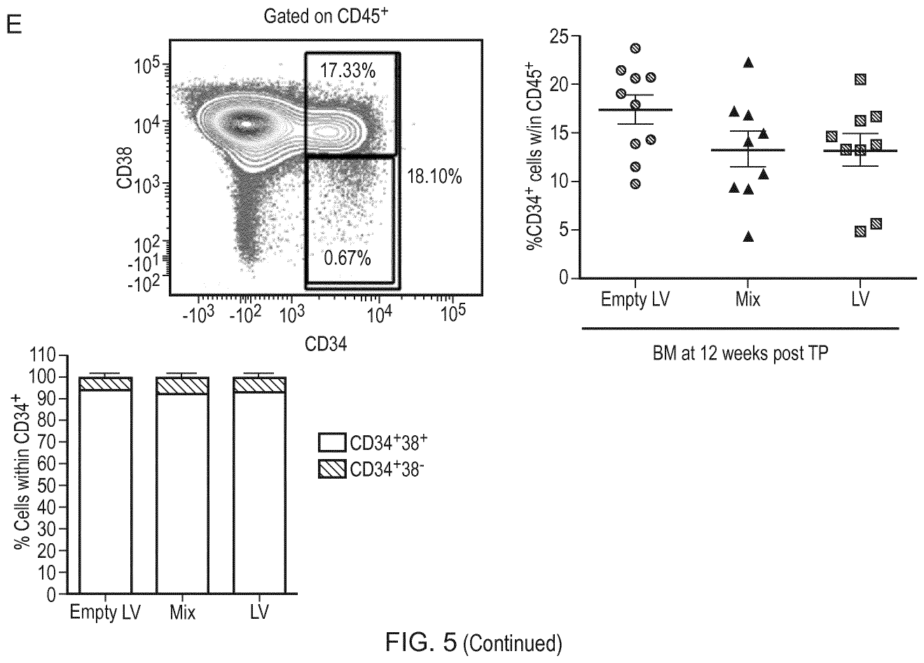

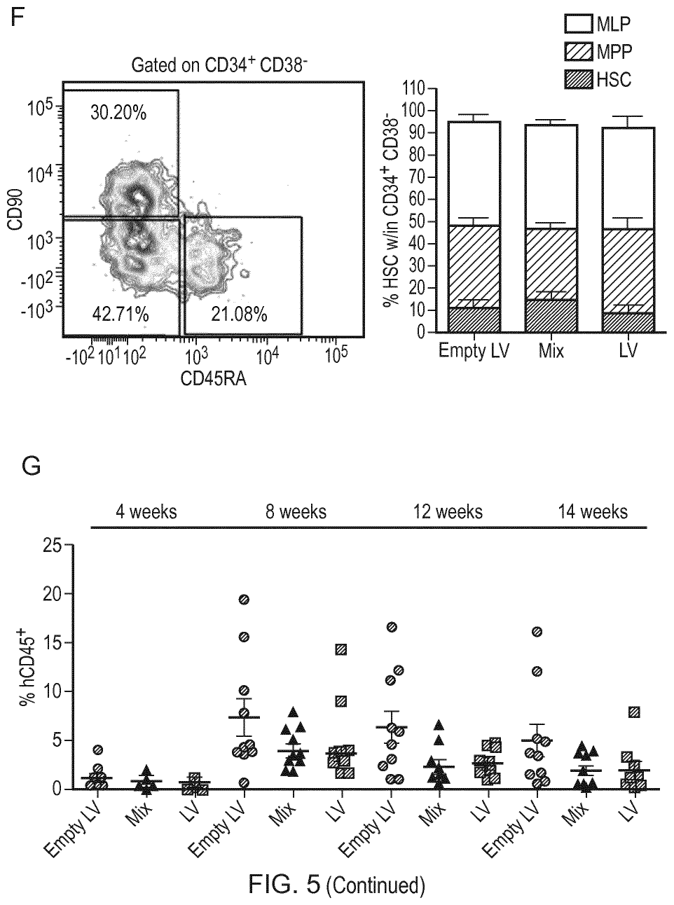

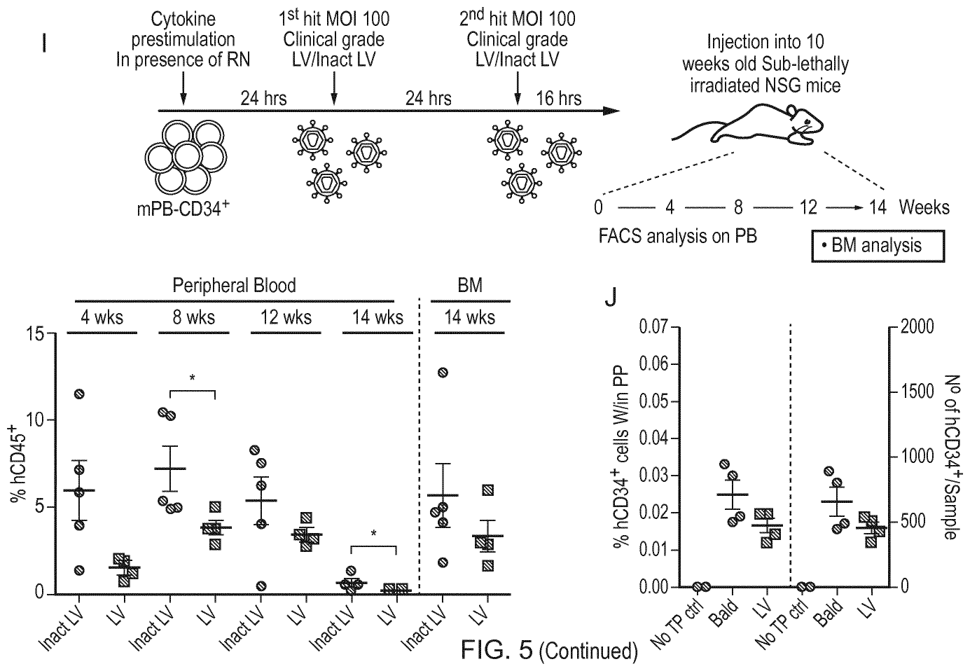

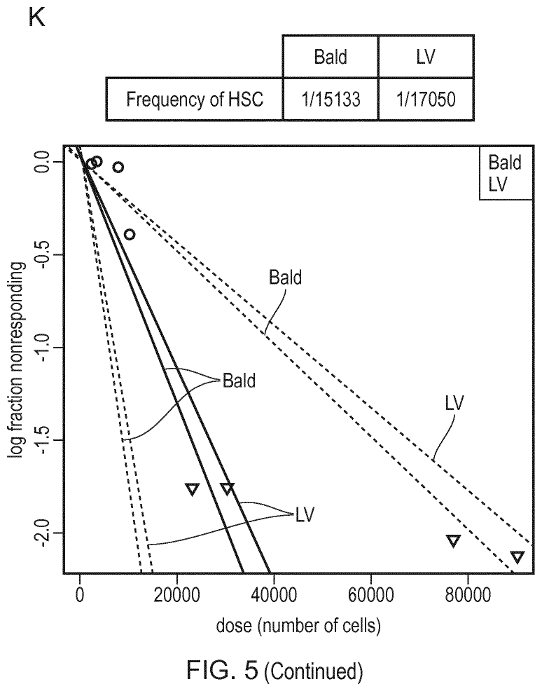

FIG. 15. Liquid culture, CFU and in vivo transduction of primary and secondary transplants. (A) Transduction efficiency at five days, (B) VCN at 14 days after (C) p21 mRNA induction (D) and Annexin V staining at 48 hours after transduction of the cells that were transplanted in NSG Mice (FIG. 5). Results are in mean±SEM of two independent experiments. (E) CB-CD34+ cells were plated in semisolid, cytokine-containing CFC medium. Colonies were scored after 14 days. Mean±SEM of total colonies per 800 transduced or bald exposed cells per subpopulation (n=4). (F) Percentage of the GFP positive cells within the human CD45+ cells in the peripheral blood over time, spleen and BM at the moment of sacrifice of (upper panel) primary or (lower panel) secondary mice. (G) VCN retrieved from the liquid culture of mPB-CD34+14 days after transduction with the two-hit clinical standard protocol as described in FIG. 5I. (H) Percentage of the Myeloid (CD13+), lymphoid (CD19+B, CD3+ T) cells within the human CD45+ cells in the peripheral blood over time, spleen and BM at the end of the experiment of the primary or secondary mice. (I) Frequencies of CD34+38+ and CD34+CD38-within the human CD34+ cells retrieved from the bone marrow of secondary recipients at the end of the experiment. (J) Percentages of human CD45+ cells (Left Panel) detected in the Bone marrow of NSG mice at 16 weeks after the transplant in the Limiting Dilution Assay (LDA) experiment, and (Right Panel) the real number of CB-CD34+ cells injected in the mice for the same experiment. (K) Interferon stimulated genes (Left Panel) mRNA levels in CB-CD34+ 48 hours after the transduction with an MOI 100 of PGK-SIN RV vector in presence of KU55933 ATM inhibitor, (Right Panel) cell proliferation assay in CB CD34+ 2 days after the transduction with MOI 100 of PGK LV in DMSO or KU55933 or MOI 100 of PGK RV or empty RV as control.

FIG. 16. In vivo of ATM inhibition. (A) Myeloid (CD13+) and lymphoid (CD19+B, CD3+ T) cells and (B) GFP+ cells within the human CD45+ cells were monitored in the peripheral blood over time (D=DMSO; and K=KU55933). Results are the mean±SEM of Bald DMSO (n=8), LV DMSO (n=10), and Bald KU55933 (n=11) and LV KU5933 (n=11) NSG mice.

### DETAILED DESCRIPTION OF THE INVENTION

Various preferred features and embodiments of the present invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O′D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### Cell Survival and Engraftment

The term “survival” refers to the ability of the haematopoietic stem and/or progenitor cells to remain alive (e.g. not die or become apoptotic) during in vitro or ex vivo culture. Haematopoietic stem and/or progenitor cells may, for example, undergo increased apoptosis following transduction with a viral vector during cell culture; thus, the surviving cells may have avoided apoptosis and/or cell death.

Cell survival may be readily analysed by the skilled person. For example, the numbers of live, dead and/or apoptotic cells in a cell culture may be quantified at the beginning of culture and/or following culture for a period of time (e.g. about 6 or 12 hours, or 1, 2, 3, 4, 5, 6, 7 or more days; preferably, the period of time begins with the transduction of the cells with a viral vector). The effect of an agent, such as an inhibitor of the invention, on cell survival may be assessed by comparing the numbers and/or percentages of live, dead and/or apoptotic cells at the beginning and/or end of the culture period between experiments carried out in the presence and absence of the agent, but under otherwise substantially identical conditions.

Cell numbers and/or percentages in certain states (e.g. live, dead or apoptotic cells) may be quantified using any of a number of methods known in the art, including use of haemocytometers, automated cell counters, flow cytometers and fluorescence activated cell sorting machines. These techniques may enable distinguishing between live, dead and/or apoptotic cells. In addition or in the alternative, apoptotic cells may be detected using readily available apoptosis assays (e.g. assays based on the detection of phosphatidylserine (PS) on the cell membrane surface, such as through use of Annexin V, which binds to exposed PS; apoptotic cells may be quantified through use of fluorescently-labelled Annexin V), which may be used to complement other techniques.

The term “engraftment” refers to the ability of the haematopoietic stem and/or progenitor cells to populate and survive in a subject following their transplantation, i.e. in the short and/or long term after transplantation. For example, engraftment may refer to the number and/or percentages of haematopoietic cells descended from the transplanted haematopoietic stem and/or progenitor cells (e.g. graft-derived cells) that are detected about 1 day to 24 weeks, 1 day to 10 weeks, or 1-30 days or 10-30 days after transplantation. In the xenograft model of human haematopoietic stem and/or progenitor cell engraftment and repopulation, engraftment may be evaluated in the peripheral blood as the percentage of cells deriving from the human xenograft (e.g. positive for the CD45 surface marker), for example. In one embodiment, engraftment is assessed at about 1, 2, 3, 4, 5, 6, 7, 8, 9, 10, 15, 20, 25 or 30 days after transplantation. In another embodiment, engraftment is assessed at about 4, 5, 6, 7, 8, 9, 10, 11, 12, 13, 14, 15, 16, 17, 18, 19, 20, 21, 22, 23 or 24 weeks after transplantation. In another embodiment, engraftment is assessed at about 16-24 weeks, preferably 20 weeks, after transplantation.

Engraftment may be readily analysed by the skilled person. For example, the transplanted haematopoietic stem and/or progenitor cells may be engineered to comprise a marker (e.g. a reporter protein, such as a fluorescent protein), which can be used to quantify the graft-derived cells. Samples for analysis may be extracted from relevant tissues and analysed ex vivo (e.g. using flow cytometry).

### P53 Activation

The term “p53 activation” refers to an increase in the activity of p53, for example through a post-translational modification of the p53 protein. Example post-translational modifications include phosphorylation, acetylation and methylation, and are described in Kruse, J. P. et al. (2008) SnapShot: p53 Posttranslational Modifications Cell 133:930-931. In the context of the invention, the p53 activation preferably results from phosphorylation of p53, particularly preferably at amino acid Serine 15.

Methods for analysing such post-translational modifications are known in the art (example methods for analysing kinase activity are disclosed herein, further methods include, for example, mass spectrometry- and antibody recognition-based methods).

An example amino acid sequence of p53, which may be used to provide an amino acid numbering convention, is:

MEEPQSDPSVEPPLSQETFSDLWKLLPENNVLSPLPSQAMDDLMLSPDDI
EQWFTEDPGPDEAPRMPEAAPPVAPAPAAPTPAAPAPAPSWPLSSSVPSQ
KTYQGSYGFRLGFLHSGTAKSVTCTYSPALNKMFCQLAKTCPVQLWVDST
PPPGTRVRAMAIYKQSQHMTEVVRRCPHHERCSDSDGLAPPQHLIRVEGN
LRVEYLDDRNTFRHSVVVPYEPPEVGSDCTTIHYNYMCNSSCMGGMNRRP
ILTIITLEDSSGNLLGRNSFEVRVCACPGRDRRTEEENLRKKGEPHHELP
PGSTKRALPNNTSSSPQPKKKPLDGEYFTLQIRGRERFEMFRELNEALEL
KDAQAGKEPGGSRAHSSHLKSKKGQSTSRHKKLMFKTEGPDSD
(SEQ ID NO: 1; NCBI Accession No. 000537.3)

### Ataxia Telangiectasia Mutated (ATM) Kinase

Ataxia telangiectasia mutated (ATM) kinase (also known as “ataxia telangiectasia mutated”) is a serine/threonine kinase which is recruited to and activated by double-strand DNA breaks. ATM kinase is known to phosphorylate a number of proteins (including p53, CHK2, BRCA1, NBS1 and H2AX) that initiate activation of the DNA damage checkpoint, leading to cell cycle arrest, DNA repair or apoptosis.

In one embodiment, the ATM kinase is human ATM kinase.

In one embodiment, the amino acid sequence of ATM kinase is the sequence deposited under NCBI Accession No. NP_000042.3.

An example amino acid sequence of the ATM kinase is:

(SEQ ID NO: 2)
MSLVLNDLLICCRQLEHDRATERKKEVEKFKRLIRDPETIKHLDRHSDSKQGKYLNWDAVFRFLQKYIQKET
ECLRIAKPNVSASTQASRQKKMQEISSLVKYFIKCANRRAPRLKCQELLNYIMDTVKDSSNGAIYGADCSNI
LLKDILSVRKYWCEISQQQWLELFSVYFRLYLKPSQDVHRVLVARIIHAVTKGCCSQTDGLNSKFLDFFSKA
IQCARQEKSSSGLNHILAALTIFLKTLAVNFRIRVCELGDEILPTLLYIWTQHRLNDSLKEVIIELFQLQIY
IHHPKGAKTQEKGAYESTKWRSILYNLYDLLVNEISHIGSRGKYSSGFRNIAVKENLIELMADICHQVFNED
TRSLEISQSYTTTQRESSDYSVPCKRKKIELGWEVIKDHLQKSQNDFDLVPWLQIATQLISKYPASLPNCEL
SPLLMILSQLLPQQRHGERTPYVLRCLTEVALCQDKRSNLESSQKSDLLKLWNKIWCITFRGISSEQIQAEN
FGLLGAIIQGSLVEVDREFWKLFTGSACRPSCPAVCCLTLALTTSIVPGTVKMGIEQNMCEVNRSFSLKESI
MKWLLFYQLEGDLENSTEVPPILHSNFPHLVLEKILVSLTMKNCKAAMNFFQSVPECEHHQKDKEELSFSEV
EELFLQTTFDKMDFLTIVRECGIEKHQSSIGFSVHQNLKESLDRCLLGLSEQLLNNYSSEITNSETLVRCSR
LLVGVLGCYCYMGVIAEEEAYKSELFQKAKSLMQCAGESITLFKNKTNEEFRIGSLRNMMQLCTRCLSNCTK
KSPNKIASGFFLRLLTSKLMNDIADICKSLASFIKKPFDRGEVESMEDDTNGNLMEVEDQSSMNLFNDYPDS
SVSDANEPGESQSTIGAINPLAEEYLSKQDLLFLDMLKFLCLCVTTAQTNTVSFRAADIRRKLLMLIDSSTL
EPTKSLHLHMYLMLLKELPGEEYPLPMEDVLELLKPLSNVCSLYRRDQDVCKTILNHVLHVVKNLGQSNMDS
ENTRDAQGQFLTVIGAFWHLTKERKYIFSVRMALVNCLKTLLEADPYSKWAILNVMGKDFPVNEVFTQFLAD
NHHQVRMLAAESINRLFQDTKGDSSRLLKALPLKLQQTAFENAYLKAQEGMREMSHSAENPETLDEIYNRKS
VLLTLIAVVLSCSPICEKQALFALCKSVKENGLEPHLVKKVLEKVSETFGYRRLEDFMASHLDYLVLEWLNL
QDTEYNLSSFPFILLNYTNIEDFYRSCYKVLIPHLVIRSHFDEVKSIANQIQEDWKSLLTDCFPKILVNILP
YFAYEGTRDSGMAQQRETATKVYDMLKSENLLGKQIDHLFISNLPEIVVELLMTLHEPANSSASQSTDLCDF
SGDLDPAPNPPHFPSHVIKATFAYISNCHKTKLKSILEILSKSPDSYQKILLAICEQAAETNNVYKKHRILK
IYHLFVSLLLKDIKSGLGGAWAFVLRDVIYTLIHYINQRPSCIMDVSLRSFSLCCDLLSQVCQTAVTYCKDA
LENHLHVIVGTLIPLVYEQVEVQKQVLDLLKYLVIDNKDNENLYITIKLLDPFPDHVVFKDLRITQQKIKYS
RGPFSLLEEINHFLSVSVYDALPLTRLEGLKDLRRQLELHKDQMVDIMRASQDNPQDGIMVKLVVNLLQLSK
MAINHTGEKEVLEAVGSCLGEVGPIDFSTIAIQHSKDASYTKALKLFEDKELQWTFIMLTYLNNTLVEDCVK
VRSAAVTCLKNILATKTGHSFWEIYKMTTDPMLAYLQPFRTSRKKFLEVPRFDKENPFEGLDDINLWIPLSE
NHDIWIKTLTCAFLDSGGTKCEILQLLKPMCEVKTDFCQTVLPYLIHDILLQDTNESWRNLLSTHVQGFFTS
CLRHFSQTSRSTTPANLDSESEHFFRCCLDKKSQRTMLAVVDYMRRQKRPSSGTIFNDAFWLDLNYLEVAKV
AQSCAAHFTALLYAEIYADKKSMDDQEKRSLAFEEGSQSTTISSLSEKSKEETGISLQDLLLEIYRSIGEPD
SLYGCGGGKMLQPITRLRTYEHEAMWGKALVTYDLETAIPSSTRQAGIIQALQNLGLCHILSVYLKGLDYEN
KDWCPELEELHYQAAWRNMQWDHCTSVSKEVEGTSYHESLYNALQSLRDREFSTFYESLKYARVKEVEEMCK
RSLESVYSLYPTLSRLQAIGELESIGELFSRSVTHRQLSEVYIKWQKHSQLLKDSDFSFQEPIMALRTVILE
ILMEKEMDNSQRECIKDILTKHLVELSILARTFKNTQLPERAIFQIKQYNSVSCGVSEWQLEEAQVFWAKKE
QSLALSILKQMIKKLDASCAANNPSLKLTYTECLRVCGNWLAETCLENPAVIMQTYLEKAVEVAGNYDGESS
DELRNGKMKAFLSLARFSDTQYQRIENYMKSSEFENKQALLKRAKEEVGLLREHKIQTNRYTVKVQRELELD
ELALRALKEDRKRFLCKAVENYINCLLSGEEHDMWVFRLCSLWLENSGVSEVNGMMKRDGMKIPTYKFLPLM
YQLAARMGTKMMGGLGFHEVLNNLISRISMDHPHHTLFIILALANANRDEFLTKPEVARRSRITKNVPKQSS
QLDEDRTEAANRIICTIRSRRPQMVRSVEALCDAYIILANLDATQWKTQRKGINIPADQPITKLKNLEDVVV
PTMEIKVDHTGEYGNLVTIQSFKAEFRLAGGVNLPKIIDCVGSDGKERRQLVKGRDDLRQDAVMQQVFQMCN
TLLQRNTETRKRKLTICTYKVVPLSQRSGVLEWCTGTVPIGEFLVNNEDGAHKRYRPNDFSAFQCQKKMMEV
QKKSFEEKYEVFMDVCQNFQPVFRYFCMEKFLDPAIWFEKRLAYTRSVATSSIVGYILGLGDRHVQNILINE
QSAELVHIDLGVAFEQGKILPTPETVPFRLTRDIVDGMGITGVEGVFRRCCEKTMEVMRNSQETLLTIVEVL
LYDPLFDWTMNPLKALYLQQRPEDETELHPTLNADDQECKRNLSDIDQSFNKVAERVLMRLQEKLKGVEEGT
VLSVGGQVNLLIQQAIDPKNLSRLFPGWKAWV

An example nucleic acid sequence encoding the ATM kinase is:

ATGAGTCTAGTACTTAATGATCTGCTTATCTGCTGCCGTCAACTAGAACATGATAGAGCTACAGAACGAA
AGAAAGAAGTTGAGAAATTTAAGCGCCTGATTCGAGATCCTGAAACAATTAAACATCTAGATCGGCATTC
AGATTCCAAACAAGGAAAATATTTGAATTGGGATGCTGTTTTTAGATTTTTACAGAAATATATTCAGAAA
GAAACAGAATGTCTGAGAATAGCAAAACCAAATGTATCAGCCTCAACACAAGCCTCCAGGCAGAAAAAGA
TGCAGGAAATCAGTAGTTTGGTCAAATACTTCATCAAATGTGCAAACAGAAGAGCACCTAGGCTAAAATG
TCAAGAACTCTTAAATTATATCATGGATACAGTGAAAGATTCATCTAATGGTGCTATTTACGGAGCTGAT
TGTAGCAACATACTACTCAAAGACATTCTTTCTGTGAGAAAATACTGGTGTGAAATATCTCAGCAACAGT
GGTTAGAATTGTTCTCTGTGTACTTCAGGCTCTATCTGAAACCTTCACAAGATGTTCATAGAGTTTTAGT
GGCTAGAATAATTCATGCTGTTACCAAAGGATGCTGTTCTCAGACTGACGGATTAAATTCCAAATTTTTG
GACTTTTTTTCCAAGGCTATTCAGTGTGCGAGACAAGAAAAGAGCTCTTCAGGTCTAAATCATATCTTAG
CAGCTCTTACTATCTTCCTCAAGACTTTGGCTGTCAACTTTCGAATTCGAGTGTGTGAATTAGGAGATGA
AATTCTTCCCACTTTGCTTTATATTTGGACTCAACATAGGCTTAATGATTCTTTAAAAGAAGTCATTATT
GAATTATTTCAACTGCAAATTTATATCCATCATCCGAAAGGAGCCAAAACCCAAGAAAAAGGTGCTTATG
AATCAACAAAATGGAGAAGTATTTTATACAACTTATATGATCTGCTAGTGAATGAGATAAGTCATATAGG
AAGTAGAGGAAAGTATTCTTCAGGATTTCGTAATATTGCCGTCAAAGAAAATTTGATTGAATTGATGGCA
GATATCTGTCACCAGGTTTTTAATGAAGATACCAGATCCTTGGAGATTTCTCAATCTTACACTACTACAC
AAAGAGAATCTAGTGATTACAGTGTCCCTTGCAAAAGGAAGAAAATAGAACTAGGCTGGGAAGTAATAAA
AGATCACCTTCAGAAGTCACAGAATGATTTTGATCTTGTGCCTTGGCTACAGATTGCAACCCAATTAATA
TCAAAGTATCCTGCAAGTTTACCTAACTGTGAGCTGTCTCCATTACTGATGATACTATCTCAGCTTCTAC
CCCAACAGCGACATGGGGAACGTACACCATATGTGTTACGATGCCTTACGGAAGTTGCATTGTGTCAAGA
CAAGAGGTCAAACCTAGAAAGCTCACAAAAGTCAGATTTATTAAAACTCTGGAATAAAATTTGGTGTATT
ACCTTTCGTGGTATAAGTTCTGAGCAAATACAAGCTGAAAACTTTGGCTTACTTGGAGCCATAATTCAGG
GTAGTTTAGTTGAGGTTGACAGAGAATTCTGGAAGTTATTTACTGGGTCAGCCTGCAGACCTTCATGTCC
TGCAGTATGCTGTTTGACTTTGGCACTGACCACCAGTATAGTTCCAGGAACGGTAAAAATGGGAATAGAG
CAAAATATGTGTGAAGTAAATAGAAGCTTTTCTTTAAAGGAATCAATAATGAAATGGCTCTTATTCTATC
AGTTAGAGGGTGACTTAGAAAATAGCACAGAAGTGCCTCCAATTCTTCACAGTAATTTTCCTCATCTTGT
ACTGGAGAAAATTCTTGTGAGTCTCACTATGAAAAACTGTAAAGCTGCAATGAATTTTTTCCAAAGCGTG
CCAGAATGTGAACACCACCAAAAAGATAAAGAAGAACTTTCATTCTCAGAAGTAGAAGAACTATTTCTTC
AGACAACTTTTGACAAGATGGACTTTTTAACCATTGTGAGAGAATGTGGTATAGAAAAGCACCAGTCCAG
TATTGGCTTCTCTGTCCACCAGAATCTCAAGGAATCACTGGATCGCTGTCTTCTGGGATTATCAGAACAG
CTTCTGAATAATTACTCATCTGAGATTACAAATTCAGAAACTCTTGTCCGGTGTTCACGTCTTTTGGTGG
GTGTCCTTGGCTGCTACTGTTACATGGGTGTAATAGCTGAAGAGGAAGCATATAAGTCAGAATTATTCCA
GAAAGCCAAGTCTCTAATGCAATGTGCAGGAGAAAGTATCACTCTGTTTAAAAATAAGACAAATGAGGAA
TTCAGAATTGGTTCCTTGAGAAATATGATGCAGCTATGTACACGTTGCTTGAGCAACTGTACCAAGAAGA
GTCCAAATAAGATTGCATCTGGCTTTTTCCTGCGATTGTTAACATCAAAGCTAATGAATGACATTGCAGA
TATTTGTAAAAGTTTAGCATCCTTCATCAAAAAGCCATTTGACCGTGGAGAAGTAGAATCAATGGAAGAT
GATACTAATGGAAATCTAATGGAGGTGGAGGATCAGTCATCCATGAATCTATTTAACGATTACCCTGATA
GTAGTGTTAGTGATGCAAACGAACCTGGAGAGAGCCAAAGTACCATAGGTGCCATTAATCCTTTAGCTGA
AGAATATCTGTCAAAGCAAGATCTACTTTTCTTAGACATGCTCAAGTTCTTGTGTTTGTGTGTAACTACT
GCTCAGACCAATACTGTGTCCTTTAGGGCAGCTGATATTCGGAGGAAATTGTTAATGTTAATTGATTCTA
GCACGCTAGAACCTACCAAATCCCTCCACCTGCATATGTATCTAATGCTTTTAAAGGAGCTTCCTGGAGA
AGAGTACCCCTTGCCAATGGAAGATGTTCTTGAACTTCTGAAACCACTATCCAATGTGTGTTCTTTGTAT
CGTCGTGACCAAGATGTTTGTAAAACTATTTTAAACCATGTCCTTCATGTAGTGAAAAACCTAGGTCAAA
GCAATATGGACTCTGAGAACACAAGGGATGCTCAAGGACAGTTTCTTACAGTAATTGGAGCATTTTGGCA
TCTAACAAAGGAGAGGAAATATATATTCTCTGTAAGAATGGCCCTAGTAAATTGCCTTAAAACTTTGCTT
GAGGCTGATCCTTATTCAAAATGGGCCATTCTTAATGTAATGGGAAAAGACTTTCCTGTAAATGAAGTAT
TTACACAATTTCTTGCTGACAATCATCACCAAGTTCGCATGTTGGCTGCAGAGTCAATCAATAGATTGTT
CCAGGACACGAAGGGAGATTCTTCCAGGTTACTGAAAGCACTTCCTTTGAAGCTTCAGCAAACAGCTTTT
GAAAATGCATACTTGAAAGCTCAGGAAGGAATGAGAGAAATGTCCCATAGTGCTGAGAACCCTGAAACTT
TGGATGAAATTTATAATAGAAAATCTGTTTTACTGACGTTGATAGCTGTGGTTTTATCCTGTAGCCCTAT
CTGCGAAAAACAGGCTTTGTTTGCCCTGTGTAAATCTGTGAAAGAGAATGGATTAGAACCTCACCTTGTG
AAAAAGGTTTTAGAGAAAGTTTCTGAAACTTTTGGATATAGACGTTTAGAAGACTTTATGGCATCTCATT
TAGATTATCTGGTTTTGGAATGGCTAAATCTTCAAGATACTGAATACAACTTATCTTCTTTTCCTTTTAT
TTTATTAAACTACACAAATATTGAGGATTTCTATAGATCTTGTTATAAGGTTTTGATTCCACATCTGGTG
ATTAGAAGTCATTTTGATGAGGTGAAGTCCATTGCTAATCAGATTCAAGAGGACTGGAAAAGTCTTCTAA
CAGACTGCTTTCCAAAGATTCTTGTAAATATTCTTCCTTATTTTGCCTATGAGGGTACCAGAGACAGTGG
GATGGCACAGCAAAGAGAGACTGCTACCAAGGTCTATGATATGCTTAAAAGTGAAAACTTATTGGGAAAA
CAGATTGATCACTTATTCATTAGTAATTTACCAGAGATTGTGGTGGAGTTATTGATGACGTTACATGAGC
CAGCAAATTCTAGTGCCAGTCAGAGCACTGACCTCTGTGACTTTTCAGGGGATTTGGATCCTGCTCCTAA
TCCACCTCATTTTCCATCGCATGTGATTAAAGCAACATTTGCCTATATCAGCAATTGTCATAAAACCAAG
TTAAAAAGCATTTTAGAAATTCTTTCCAAAAGCCCTGATTCCTATCAGAAAATTCTTCTTGCCATATGTG
AGCAAGCAGCTGAAACAAATAATGTTTATAAGAAGCACAGAATTCTTAAAATATATCACCTGTTTGTTAG
TTTATTACTGAAAGATATAAAAAGTGGCTTAGGAGGAGCTTGGGCCTTTGTTCTTCGAGACGTTATTTAT
ACTTTGATTCACTATATCAACCAAAGGCCTTCTTGTATCATGGATGTGTCATTACGTAGCTTCTCCCTTT
GTTGTGACTTATTAAGTCAGGTTTGCCAGACAGCCGTGACTTACTGTAAGGATGCTCTAGAAAACCATCT
TCATGTTATTGTTGGTACACTTATACCCCTTGTGTATGAGCAGGTGGAGGTTCAGAAACAGGTATTGGAC
TTGTTGAAATACTTAGTGATAGATAACAAGGATAATGAAAACCTCTATATCACGATTAAGCTTTTAGATC
CTTTTCCTGACCATGTTGTTTTTAAGGATTTGCGTATTACTCAGCAAAAAATCAAATACAGTAGAGGACC
CTTTTCACTCTTGGAGGAAATTAACCATTTTCTCTCAGTAAGTGTTTATGATGCACTTCCATTGACAAGA
CTTGAAGGACTAAAGGATCTTCGAAGACAACTGGAACTACATAAAGATCAGATGGTGGACATTATGAGAG
CTTCTCAGGATAATCCGCAAGATGGGATTATGGTGAAACTAGTTGTCAATTTGTTGCAGTTATCCAAGAT
GGCAATAAACCACACTGGTGAAAAAGAAGTTCTAGAGGCTGTTGGAAGCTGCTTGGGAGAAGTGGGTCCT
ATAGATTTCTCTACCATAGCTATACAACATAGTAAAGATGCATCTTATACCAAGGCCCTTAAGTTATTTG
AAGATAAAGAACTTCAGTGGACCTTCATAATGCTGACCTACCTGAATAACACACTGGTAGAAGATTGTGT
CAAAGTTCGATCAGCAGCTGTTACCTGTTTGAAAAACATTTTAGCCACAAAGACTGGACATAGTTTCTGG
GAGATTTATAAGATGACAACAGATCCAATGCTGGCCTATCTACAGCCTTTTAGAACATCAAGAAAAAAGT
TTTTAGAAGTACCCAGATTTGACAAAGAAAACCCTTTTGAAGGCCTGGATGATATAAATCTGTGGATTCC
TCTAAGTGAAAATCATGACATTTGGATAAAGACACTGACTTGTGCTTTTTTGGACAGTGGAGGCACAAAA
TGTGAAATTCTTCAATTATTAAAGCCAATGTGTGAAGTGAAAACTGACTTTTGTCAGACTGTACTTCCAT
ACTTGATTCATGATATTTTACTCCAAGATACAAATGAATCATGGAGAAATCTGCTTTCTACACATGTTCA
GGGATTTTTCACCAGCTGTCTTCGACACTTCTCGCAAACGAGCCGATCCACAACCCCTGCAAACTTGGAT
TCAGAGTCAGAGCACTTTTTCCGATGCTGTTTGGATAAAAAATCACAAAGAACAATGCTTGCTGTTGTGG
ACTACATGAGAAGACAAAAGAGACCTTCTTCAGGAACAATTTTTAATGATGCTTTCTGGCTGGATTTAAA
TTATCTAGAAGTTGCCAAGGTAGCTCAGTCTTGTGCTGCTCACTTTACAGCTTTACTCTATGCAGAAATC
TATGCAGATAAGAAAAGTATGGATGATCAAGAGAAAAGAAGTCTTGCATTTGAAGAAGGAAGCCAGAGTA
CAACTATTTCTAGCTTGAGTGAAAAAAGTAAAGAAGAAACTGGAATAAGTTTACAGGATCTTCTCTTAGA
AATCTACAGAAGTATAGGGGAGCCAGATAGTTTGTATGGCTGTGGTGGAGGGAAGATGTTACAACCCATT
ACTAGACTACGAACATATGAACACGAAGCAATGTGGGGCAAAGCCCTAGTAACATATGACCTCGAAACAG
CAATCCCCTCATCAACACGCCAGGCAGGAATCATTCAGGCCTTGCAGAATTTGGGACTCTGCCATATTCT
TTCCGTCTATTTAAAAGGATTGGATTATGAAAATAAAGACTGGTGTCCTGAACTAGAAGAACTTCATTAC
CAAGCAGCATGGAGGAATATGCAGTGGGACCATTGCACTTCCGTCAGCAAAGAAGTAGAAGGAACCAGTT
ACCATGAATCATTGTACAATGCTCTACAATCTCTAAGAGACAGAGAATTCTCTACATTTTATGAAAGTCT
CAAATATGCCAGAGTAAAAGAAGTGGAAGAGATGTGTAAGCGCAGCCTTGAGTCTGTGTATTCGCTCTAT
CCCACACTTAGCAGGTTGCAGGCCATTGGAGAGCTGGAAAGCATTGGGGAGCTTTTCTCAAGATCAGTCA
CACATAGACAACTCTCTGAAGTATATATTAAGTGGCAGAAACACTCCCAGCTTCTCAAGGACAGTGATTT
TAGTTTTCAGGAGCCTATCATGGCTCTACGCACAGTCATTTTGGAGATCCTGATGGAAAAGGAAATGGAC
AACTCACAAAGAGAATGTATTAAGGACATTCTCACCAAACACCTTGTAGAACTCTCTATACTGGCCAGAA
CTTTCAAGAACACTCAGCTCCCTGAAAGGGCAATATTTCAAATTAAACAGTACAATTCAGTTAGCTGTGG
AGTCTCTGAGTGGCAGCTGGAAGAAGCACAAGTATTCTGGGCAAAAAAGGAGCAGAGTCTTGCCCTGAGT
ATTCTCAAGCAAATGATCAAGAAGTTGGATGCCAGCTGTGCAGCGAACAATCCCAGCCTAAAACTTACAT
ACACAGAATGTCTGAGGGTTTGTGGCAACTGGTTAGCAGAAACGTGCTTAGAAAATCCTGCGGTCATCAT
GCAGACCTATCTAGAAAAGGCAGTAGAAGTTGCTGGAAATTATGATGGAGAAAGTAGTGATGAGCTAAGA
AATGGAAAAATGAAGGCATTTCTCTCATTAGCCCGGTTTTCAGATACTCAATACCAAAGAATTGAAAACT
ACATGAAATCATCGGAATTTGAAAACAAGCAAGCTCTCCTGAAAAGAGCCAAAGAGGAAGTAGGTCTCCT
TAGGGAACATAAAATTCAGACAAACAGATACACAGTAAAGGTTCAGCGAGAGCTGGAGTTGGATGAATTA
GCCCTGCGTGCACTGAAAGAGGATCGTAAACGCTTCTTATGTAAAGCAGTTGAAAATTATATCAACTGCT
TATTAAGTGGAGAAGAACATGATATGTGGGTATTCCGACTTTGTTCCCTCTGGCTTGAAAATTCTGGAGT
TTCTGAAGTCAATGGCATGATGAAGAGAGACGGAATGAAGATTCCAACATATAAATTTTTGCCTCTTATG
TACCAATTGGCTGCTAGAATGGGGACCAAGATGATGGGAGGCCTAGGATTTCATGAAGTCCTCAATAATC
TAATCTCTAGAATTTCAATGGATCACCCCCATCACACTTTGTTTATTATACTGGCCTTAGCAAATGCAAA
CAGAGATGAATTTCTGACTAAACCAGAGGTAGCCAGAAGAAGCAGAATAACTAAAAATGTGCCTAAACAA
AGCTCTCAGCTTGATGAGGATCGAACAGAGGCTGCAAATAGAATAATATGTACTATCAGAAGTAGGAGAC
CTCAGATGGTCAGAAGTGTTGAGGCACTTTGTGATGCTTATATTATATTAGCAAACTTAGATGCCACTCA
GTGGAAGACTCAGAGAAAAGGCATAAATATTCCAGCAGACCAGCCAATTACTAAACTTAAGAATTTAGAA
GATGTTGTTGTCCCTACTATGGAAATTAAGGTGGACCACACAGGAGAATATGGAAATCTGGTGACTATAC
AGTCATTTAAAGCAGAATTTCGCTTAGCAGGAGGTGTAAATTTACCAAAAATAATAGATTGTGTAGGTTC
CGATGGCAAGGAGAGGAGACAGCTTGTTAAGGGCCGTGATGACCTGAGACAAGATGCTGTCATGCAACAG
GTCTTCCAGATGTGTAATACATTACTGCAGAGAAACACGGAAACTAGGAAGAGGAAATTAACTATCTGTA
CTTATAAGGTGGTTCCCCTCTCTCAGCGAAGTGGTGTTCTTGAATGGTGCACAGGAACTGTCCCCATTGG
TGAATTTCTTGTTAACAATGAAGATGGTGCTCATAAAAGATACAGGCCAAATGATTTCAGTGCCTTTCAG
TGCCAAAAGAAAATGATGGAGGTGCAAAAAAAGTCTTTTGAAGAGAAATATGAAGTCTTCATGGATGTTT
GCCAAAATTTTCAACCAGTTTTCCGTTACTTCTGCATGGAAAAATTCTTGGATCCAGCTATTTGGTTTGA
GAAGCGATTGGCTTATACGCGCAGTGTAGCTACTTCTTCTATTGTTGGTTACATACTTGGACTTGGTGAT
AGACATGTACAGAATATCTTGATAAATGAGCAGTCAGCAGAACTTGTACATATAGATCTAGGTGTTGCTT
TTGAACAGGGCAAAATCCTTCCTACTCCTGAGACAGTTCCTTTTAGACTCACCAGAGATATTGTGGATGG
CATGGGCATTACGGGTGTTGAAGGTGTCTTCAGAAGATGCTGTGAGAAAACCATGGAAGTGATGAGAAAC
TCTCAGGAAACTCTGTTAACCATTGTAGAGGTCCTTCTATATGATCCACTCTTTGACTGGACCATGAATC
CTTTGAAAGCTTTGTATTTACAGCAGAGGCCGGAAGATGAAACTGAGCTTCACCCTACTCTGAATGCAGA
TGACCAAGAATGCAAACGAAATCTCAGTGATATTGACCAGAGTTTCAACAAAGTAGCTGAACGTGTCTTA
ATGAGACTACAAGAGAAACTGAAAGGAGTGGAAGAAGGCACTGTGCTCAGTGTTGGTGGACAAGTGAATT
TGCTCATACAGCAGGCCATAGACCCCAAAAATCTCAGCCGACTTTTCCCAGGATGGAAAGCTTGGGTGTG
A (SEQ ID NO: 3; NCBI Accession No. NM_000051.3)

### Ataxia Telangiectasia and Rad3-Related Protein (ATR)

Ataxia telangiectasia and Rad3-related protein (ATR), also known as serine/threonine-protein kinase ATR or FRAP-related protein 1 (FRP1), is a serine/threonine-specific kinase involved in DNA damage sensing. It may be involved in activating the DNA damage checkpoint, which leads to cell cycle arrest

In one embodiment, the ATR is human ATR.

In one embodiment, the amino acid sequence of ATR is the sequence deposited under NCBI Accession No. NP_001175.2.

An example amino acid sequence of the ATR is:

(SEQ ID NO: 4)
MGEHGLELASMIPALRELGSATPEEYNTVVQKPRQILCQFIDRILTDVNVVAVELVKKTDSQPTSVMLLDFI
QHIMKSSPLMFVNVSGSHEAKGSCIEFSNWIITRLLRIAATPSCHLLHKKICEVICSLLFLEKSKSPAIFGV
LTKELLQLFEDLVYLHRRNVMGHAVEWPVVMSRFLSQLDEHMGYLQSAPLQLMSMQNLEFIEVTLLMVLTRI
IAIVFFRRQELLLWQIGCVLLEYGSPKIKSLAISFLTELFQLGGLPAQPASTFFSSFLELLKHLVEMDTDQL
KLYEEPLSKLIKTLFPFEAEAYRNIEPVYLNMLLEKLCVMFEDGVLMRLKSDLLKAALCHLLQYFLKFVPAG
YESALQVRKVYVRNICKALLDVLGIEVDAEYLLGPLYAALKMESMEIIEEIQCQTQQENLSSNSDGISPKRR
RLSSSLNPSKRAPKQTEEIKHVDMNQKSILWSALKQKAESLQISLEYSGLKNPVIEMLEGIAVVLQLTALCT
VHCSHQNMNCRTFKDCQHKSKKKPSVVITWMSLDFYTKVLKSCRSLLESVQKLDLEATIDKVVKIYDALIYM
QVNSSFEDHILEDLCGMLSLPWIYSHSDDGCLKLTTFAANLLTLSCRISDSYSPQAQSRCVFLLTLFPRRIF
LEWRTAVYNWALQSSHEVIRASCVSGFFILLQQQNSCNRVPKILIDKVKDDSDIVKKEFASILGQLVCTLHG
MFYLTSSLTEPFSEHGHVDLFCRNLKATSQHECSSSQLKASVCKPFLFLLKKKIPSPVKLAFIDNLHHLCKH
LDFREDETDVKAVLGTLLNLMEDPDKDVRVAFSGNIKHILESLDSEDGFIKELFVLRMKEAYTHAQISRNNE
LKDTLILTTGDIGRAAKGDLVPFALLHLLHCLLSKSASVSGAAYTEIRALVAAKSVKLQSFFSQYKKPICQF
LVESLHSSQMTALPNTPCQNADVRKQDVAHQREMALNTLSEIANVFDFPDLNRFLTRTLQVLLPDLAAKASP
AASALIRTLGKQLNVNRREILINNFKYIFSHLVCSCSKDELERALHYLKNETEIELGSLLRQDFQGLHNELL
LRIGEHYQQVFNGLSILASFASSDDPYQGPRDIISPELMADYLQPKLLGILAFFNMQLLSSSVGIEDKKMAL
NSLMSLMKLMGPKHVSSVRVKMMTTLRTGLRFKDDFPELCCRAWDCFVRCLDHACLGSLLSHVIVALLPLIH
IQPKETAAIFHYLIIENRDAVQDFLHEIYFLPDHPELKKIKAVLQEYRKETSESTDLQTTLQLSMKAIQHEN
VDVRIHALTSLKETLYKNQEKLIKYATDSETVEPIISQLVTVLLKGCQDANSQARLLCGECLGELGAIDPGR
LDFSTTETQGKDFTFVTGVEDSSFAYGLLMELTRAYLAYADNSRAQDSAAYAIQELLSIYDCREMETNGPGH
QLWRRFPEHVREILEPHLNTRYKSSQKSTDWSGVKKPIYLSKLGSNFAEWSASWAGYLITKVRHDLASKIFT
CCSIMMKHDFKVTIYLLPHILVYVLLGCNQEDQQEVYAEIMAVLKHDDQHTINTQDIASDLCQLSTQTVFSM
LDHLTQWARHKFQALKAEKCPHSKSNRNKVDSMVSTVDYEDYQSVTRFLDLIPQDTLAVASFRSKAYTRAVM
HFESFITEKKQNIQEHLGFLQKLYAAMHEPDGVAGVSAIRKAEPSLKEQILEHESLGLLRDATACYDRAIQL
EPDQIIHYHGVVKSMLGLGQLSTVITQVNGVHANRSEWTDELNTYRVEAAWKLSQWDLVENYLAADGKSTTW
SVRLGQLLLSAKKRDITAFYDSLKLVRAEQIVPLSAASFERGSYQRGYEYIVRLHMLCELEHSIKPLFQHSP
GDSSQEDSLNWVARLEMTQNSYRAKEPILALRRALLSLNKRPDYNEMVGECWLQSARVARKAGHHQTAYNAL
LNAGESRLAELYVERAKWLWSKGDVHQALIVLQKGVELCFPENETPPEGKNMLIHGRAMLLVGRFMEETANF
ESNAIMKKYKDVTACLPEWEDGHFYLAKYYDKLMPMVTDNKMEKQGDLIRYIVLHFGRSLQYGNQFIYQSMP
RMLTLWLDYGTKAYEWEKAGRSDRVQMRNDLGKINKVITEHTNYLAPYQFLTAFSQLISRICHSHDEVFVVL
MEIIAKVFLAYPQQAMWMMTAVSKSSYPMRVNRCKEILNKAIHMKKSLEKFVGDATRLTDKLLELCNKPVDG
SSSTLSMSTHFKMLKKLVEEATFSEILIPLQSVMIPTLPSILGTHANHASHEPFPGHWAYIAGFDDMVEILA
SLQKPKKISLKGSDGKFYIMMCKPKDDLRKDCRLMEFNSLINKCLRKDAESRRRELHIRTYAVIPLNDECGI
IEWVNNTAGLRPILTKLYKEKGVYMTGKELRQCMLPKSAALSEKLKVFREFLLPRHPPIFHEWFLRTFPDPT
SWYSSRSAYCRSTAVMSMVGYILGLGDRHGENILFDSLTGECVHVDFNCLFNKGETFEVPEIVPFRLTHNMV
NGMGPMGTEGLFRRACEVTMRLMRDQREPLMSVLKTFLHDPLVEWSKPVKGHSKAPLNETGEVVNEKAKTHV
LDIEQRLQGVIKTRNRVTGLPLSIEGHVHYLIQEATDENLLCQMYLGWTPYM

An example nucleic acid sequence encoding the ATR is:

ATGGGGGAACATGGCCTGGAGCTGGCTTCCATGATCCCCGCCCTGCGGGAGCTGGGCAGTGCCACACCAG
AGGAATATAATACAGTTGTACAGAAGCCAAGACAAATTCTGTGTCAATTCATTGACCGGATACTTACAGA
TGTAAATGTTGTTGCTGTAGAACTTGTAAAGAAAACTGACTCTCAGCCAACCTCCGTGATGTTGCTTGAT
TTCATCCAGCATATCATGAAATCCTCCCCACTTATGTTTGTAAATGTGAGTGGAAGCCATGAGGCCAAAG
GCAGTTGTATTGAATTCAGTAATTGGATCATAACGAGACTTCTGCGGATTGCAGCAACTCCCTCCTGTCA
TTTGTTACACAAGAAAATCTGTGAAGTCATCTGTTCATTATTATTTCTTTTTAAAAGCAAGAGTCCTGCT
ATTTTTGGGGTACTCACAAAAGAATTATTACAACTTTTTGAAGACTTGGTTTACCTCCATAGAAGAAATG
TGATGGGTCATGCTGTGGAATGGCCAGTGGTCATGAGCCGATTTTTAAGTCAATTAGATGAACACATGGG
ATATTTACAATCAGCTCCTTTGCAGTTGATGAGTATGCAAAATTTAGAATTTATTGAAGTCACTTTATTA
ATGGTTCTTACTCGTATTATTGCAATTGTGTTTTTTAGAAGGCAAGAACTCTTACTTTGGCAGATAGGTT
GTGTTCTGCTAGAGTATGGTAGTCCAAAAATTAAATCCCTAGCAATTAGCTTTTTAACAGAACTTTTTCA
GCTTGGAGGACTACCAGCACAACCAGCTAGCACTTTTTTCAGCTCATTTTTGGAATTATTAAAACACCTT
GTAGAAATGGATACTGACCAATTGAAACTCTATGAAGAGCCATTATCAAAGCTGATAAAGACACTATTTC
CCTTTGAAGCAGAAGCTTATAGAAATATTGAACCTGTCTATTTAAATATGCTGCTGGAAAAACTCTGTGT
CATGTTTGAAGACGGTGTGCTCATGCGGCTTAAGTCTGATTTGCTAAAAGCAGCTTTGTGCCATTTACTG
CAGTATTTCCTTAAATTTGTGCCAGCTGGGTATGAATCTGCTTTACAAGTCAGGAAGGTCTATGTGAGAA
ATATTTGTAAAGCTCTTTTGGATGTGCTTGGAATTGAGGTAGATGCAGAGTACTTGTTGGGCCCACTTTA
TGCAGCTTTGAAAATGGAAAGTATGGAAATCATTGAGGAGATTCAATGCCAAACTCAACAGGAAAACCTC
AGCAGTAATAGTGATGGAATATCACCCAAAAGGCGTCGTCTCAGCTCGTCTCTAAACCCTTCTAAAAGAG
CACCAAAACAGACTGAGGAAATTAAACATGTGGACATGAACCAAAAGAGCATATTATGGAGTGCACTGAA
ACAGAAAGCTGAATCCCTTCAGATTTCCCTTGAATACAGTGGCCTAAAGAATCCTGTTATTGAGATGTTA
GAAGGAATTGCTGTTGTCTTACAACTGACTGCTCTGTGTACTGTTCATTGTTCTCATCAAAACATGAACT
GCCGTACTTTCAAGGACTGTCAACATAAATCCAAGAAGAAACCTTCTGTAGTGATAACTTGGATGTCATT
GGATTTTTACACAAAAGTGCTTAAGAGCTGTAGAAGTTTGTTAGAATCTGTTCAGAAACTGGACCTGGAG
GCAACCATTGATAAGGTGGTGAAAATTTATGATGCTTTGATTTATATGCAAGTAAACAGTTCATTTGAAG
ATCATATCCTGGAAGATTTATGTGGTATGCTCTCACTTCCATGGATTTATTCCCATTCTGATGATGGCTG
TTTAAAGTTGACCACATTTGCCGCTAATCTTCTAACATTAAGCTGTAGGATTTCAGATAGCTATTCACCA
CAGGCACAATCACGATGTGTGTTTCTTCTGACTCTGTTTCCAAGAAGAATATTCCTTGAGTGGAGAACAG
CAGTTTACAACTGGGCCCTGCAGAGCTCCCATGAAGTAATCCGGGCTAGTTGTGTTAGTGGATTTTTTAT
CTTATTGCAGCAGCAGAATTCTTGTAACAGAGTTCCCAAGATTCTTATAGATAAAGTCAAAGATGATTCT
GACATTGTCAAGAAAGAATTTGCTTCTATACTTGGTCAACTTGTCTGTACTCTTCACGGCATGTTTTATC
TGACAAGTTCTTTAACAGAACCTTTCTCTGAACACGGACATGTGGACCTCTTCTGTAGGAACTTGAAAGC
CACTTCTCAACATGAATGTTCATCTTCTCAACTAAAAGCTTCTGTCTGCAAGCCATTCCTTTTCCTACTG
AAAAAAAAAATACCTAGTCCAGTAAAACTTGCTTTCATAGATAATCTACATCATCTTTGTAAGCATCTTG
ATTTTAGAGAAGATGAAACAGATGTAAAAGCAGTTCTTGGAACTTTATTAAATTTAATGGAAGATCCAGA
CAAAGATGTTAGAGTGGCTTTTAGTGGAAATATCAAGCACATATTGGAATCCTTGGACTCTGAAGATGGA
TTTATAAAGGAGCTTTTTGTCTTAAGAATGAAGGAAGCATATACACATGCCCAAATATCAAGAAATAATG
AGCTGAAGGATACCTTGATTCTTACAACAGGGGATATTGGAAGGGCCGCAAAAGGAGATTTGGTACCATT
TGCACTCTTACACTTATTGCATTGTTTGTTATCCAAGTCAGCATCTGTCTCTGGAGCAGCATACACAGAA
ATTAGAGCTCTGGTTGCAGCTAAAAGTGTTAAACTGCAAAGTTTTTTCAGCCAGTATAAGAAACCCATCT
GTCAGTTTTTGGTAGAATCCCTTCACTCTAGTCAGATGACAGCACTTCCGAATACTCCATGCCAGAATGC
TGACGTGCGAAAACAAGATGTGGCTCACCAGAGAGAAATGGCTTTAAATACGTTGTCTGAAATTGCCAAC
GTTTTCGACTTTCCTGATCTTAATCGTTTTCTTACTAGGACATTACAAGTTCTACTACCTGATCTTGCTG
CCAAAGCAAGCCCTGCAGCTTCTGCTCTCATTCGAACTTTAGGAAAACAATTAAATGTCAATCGTAGAGA
GATTTTAATAAACAACTTCAAATATATTTTTTCTCATTTGGTCTGTTCTTGTTCCAAAGATGAATTAGAA
CGTGCCCTTCATTATCTGAAGAATGAAACAGAAATTGAACTGGGGAGCCTGTTGAGACAAGATTTCCAAG
GATTGCATAATGAATTATTGCTGCGTATTGGAGAACACTATCAACAGGTTTTTAATGGTTTGTCAATACT
TGCCTCATTTGCATCCAGTGATGATCCATATCAGGGCCCGAGAGATATCATATCACCTGAACTGATGGCT
GATTATTTACAACCCAAATTGTTGGGCATTTTGGCTTTTTTTAACATGCAGTTACTGAGCTCTAGTGTTG
GCATTGAAGATAAGAAAATGGCCTTGAACAGTTTGATGTCTTTGATGAAGTTAATGGGACCCAAACATGT
CAGTTCTGTGAGGGTGAAGATGATGACCACACTGAGAACTGGCCTTCGATTCAAGGATGATTTTCCTGAA
TTGTGTTGCAGAGCTTGGGACTGCTTTGTTCGCTGCCTGGATCATGCTTGTCTGGGCTCCCTTCTCAGTC
ATGTAATAGTAGCTTTGTTACCTCTTATACACATCCAGCCTAAAGAAACTGCAGCTATCTTCCACTACCT
CATAATTGAAAACAGGGATGCTGTGCAAGATTTTCTTCATGAAATATATTTTTTACCTGATCATCCAGAA
TTAAAAAAGATAAAAGCCGTTCTCCAGGAATACAGAAAGGAGACCTCTGAGAGCACTGATCTTCAGACAA
CTCTTCAGCTCTCTATGAAGGCCATTCAACATGAAAATGTCGATGTTCGTATTCATGCTCTTACAAGCTT
GAAGGAAACCTTGTATAAAAATCAGGAAAAACTGATAAAGTATGCAACAGACAGTGAAACAGTAGAACCT
ATTATCTCACAGTTGGTGACAGTGCTTTTGAAAGGTTGCCAAGATGCAAACTCTCAAGCTCGGTTGCTCT
GTGGGGAATGTTTAGGGGAATTGGGGGCGATAGATCCAGGTCGATTAGATTTCTCAACAACTGAAACTCA
AGGAAAAGATTTTACATTTGTGACTGGAGTAGAAGATTCAAGCTTTGCCTATGGATTATTGATGGAGCTA
ACAAGAGCTTACCTTGCGTATGCTGATAATAGCCGAGCTCAAGATTCAGCTGCCTATGCCATTCAGGAGT
TGCTTTCTATTTATGACTGTAGAGAGATGGAGACCAACGGCCCAGGTCACCAATTGTGGAGGAGATTTCC
TGAGCATGTTCGGGAAATACTAGAACCTCATCTAAATACCAGATACAAGAGTTCTCAGAAGTCAACCGAT
TGGTCTGGAGTAAAGAAGCCAATTTACTTAAGTAAATTGGGTAGTAACTTTGCAGAATGGTCAGCATCTT
GGGCAGGTTATCTTATTACAAAGGTTCGACATGATCTTGCCAGTAAAATTTTCACCTGCTGTAGCATTAT
GATGAAGCATGATTTCAAAGTGACCATCTATCTTCTTCCACATATTCTGGTGTATGTCTTACTGGGTTGT
AATCAAGAAGATCAGCAGGAGGTTTATGCAGAAATTATGGCAGTTCTAAAGCATGACGATCAGCATACCA
TAAATACCCAAGACATTGCATCTGATCTGTGTCAACTCAGTACACAGACTGTGTTCTCCATGCTTGACCA
TCTCACACAGTGGGCAAGGCACAAATTTCAGGCACTGAAAGCTGAGAAATGTCCACACAGCAAATCAAAC
AGAAATAAGGTAGACTCAATGGTATCTACTGTGGATTATGAAGACTATCAGAGTGTAACCCGTTTTCTAG
ACCTCATACCCCAGGATACTCTGGCAGTAGCTTCCTTTCGCTCCAAAGCATACACACGAGCTGTAATGCA
CTTTGAATCATTTATTACAGAAAAGAAGCAAAATATTCAGGAACATCTTGGATTTTTACAGAAATTGTAT
GCTGCTATGCATGAACCTGATGGAGTGGCCGGAGTCAGTGCAATTAGAAAGGCAGAACCATCTCTAAAAG
AACAGATCCTTGAACATGAAAGCCTTGGCTTGCTGAGGGATGCCACTGCTTGTTATGACAGGGCTATTCA
GCTAGAACCAGACCAGATCATTCATTATCATGGTGTAGTAAAGTCCATGTTAGGTCTTGGTCAGCTGTCT
ACTGTTATCACTCAGGTGAATGGAGTGCATGCTAACAGGTCCGAGTGGACAGATGAATTAAACACGTACA
GAGTGGAAGCAGCTTGGAAATTGTCACAGTGGGATTTGGTGGAAAACTATTTGGCAGCAGATGGAAAATC
TACAACATGGAGTGTCAGACTGGGACAGCTATTATTATCAGCCAAAAAAAGAGATATCACAGCTTTTTAT
GACTCACTGAAACTAGTGAGAGCAGAACAAATTGTACCTCTTTCAGCTGCAAGCTTTGAAAGAGGCTCCT
ACCAACGAGGATATGAATATATTGTGAGATTGCACATGTTATGTGAGTTGGAGCATAGCATCAAACCACT
TTTCCAGCATTCTCCAGGTGACAGTTCTCAAGAAGATTCTCTAAACTGGGTAGCTCGACTAGAAATGACC
CAGAATTCCTACAGAGCCAAGGAGCCTATCCTGGCTCTCCGGAGGGCTTTACTAAGCCTCAACAAAAGAC
CAGATTACAATGAAATGGTTGGAGAATGCTGGCTGCAGAGTGCCAGGGTAGCTAGAAAGGCTGGTCACCA
CCAGACAGCCTACAATGCTCTCCTTAATGCAGGGGAATCACGACTCGCTGAACTGTACGTGGAAAGGGCA
AAGTGGCTCTGGTCCAAGGGTGATGTTCACCAGGCACTAATTGTTCTTCAAAAAGGTGTTGAATTATGTT
TTCCTGAAAATGAAACCCCACCTGAGGGTAAGAACATGTTAATCCATGGTCGAGCTATGCTACTAGTGGG
CCGATTTATGGAAGAAACAGCTAACTTTGAAAGCAATGCAATTATGAAAAAATATAAGGATGTGACCGCG
TGCCTGCCAGAATGGGAGGATGGGCATTTTTACCTTGCCAAGTACTATGACAAATTGATGCCCATGGTCA
CAGACAACAAAATGGAAAAGCAAGGTGATCTCATCCGGTATATAGTTCTTCATTTTGGCAGATCTCTACA
ATATGGAAATCAGTTCATATATCAGTCAATGCCACGAATGTTAACTCTATGGCTTGATTATGGTACAAAG
GCATATGAATGGGAAAAAGCTGGCCGCTCCGATCGTGTACAAATGAGGAATGATTTGGGTAAAATAAACA
AGGTTATCACAGAGCATACAAACTATTTAGCTCCATATCAATTTTTGACTGCTTTTTCACAATTGATCTC
TCGAATTTGTCATTCTCACGATGAAGTTTTTGTTGTCTTGATGGAAATAATAGCCAAAGTATTTCTAGCC
TATCCTCAACAAGCAATGTGGATGATGACAGCTGTGTCAAAGTCATCTTATCCCATGCGTGTGAACAGAT
GCAAGGAAATCCTCAATAAAGCTATTCATATGAAAAAATCCTTAGAGAAGTTTGTTGGAGATGCAACTCG
CCTAACAGATAAGCTTCTAGAATTGTGCAATAAACCGGTTGATGGAAGTAGTTCCACATTAAGCATGAGC
ACTCATTTTAAAATGCTTAAAAAGCTGGTAGAAGAAGCAACATTTAGTGAAATCCTCATTCCTCTACAAT
CAGTCATGATACCTACACTTCCATCAATTCTGGGTACCCATGCTAACCATGCTAGCCATGAACCATTTCC
TGGACATTGGGCCTATATTGCAGGGTTTGATGATATGGTGGAAATTCTTGCTTCTCTTCAGAAACCAAAG
AAGATTTCTTTAAAAGGCTCAGATGGAAAGTTCTACATCATGATGTGTAAGCCAAAAGATGACCTGAGAA
AGGATTGTAGACTAATGGAATTCAATTCCTTGATTAATAAGTGCTTAAGAAAAGATGCAGAGTCTCGTAG
AAGAGAACTTCATATTCGAACATATGCAGTTATTCCACTAAATGATGAATGTGGGATTATTGAATGGGTG
AACAACACTGCTGGTTTGAGACCTATTCTGACCAAACTATATAAAGAAAAGGGAGTGTATATGACAGGAA
AAGAACTTCGCCAGTGTATGCTACCAAAGTCAGCAGCTTTATCTGAAAAACTCAAAGTATTCCGAGAATT
TCTCCTGCCCAGGCATCCTCCTATTTTTCATGAGTGGTTTCTGAGAACATTCCCTGATCCTACATCATGG
TACAGTAGTAGATCAGCTTACTGCCGTTCCACTGCAGTAATGTCAATGGTTGGTTATATTCTGGGGCTTG
GAGACCGTCATGGTGAAAATATTCTCTTTGATTCTTTGACTGGTGAATGCGTACATGTAGATTTCAATTG
TCTTTTCAATAAGGGAGAAACCTTTGAAGTTCCAGAAATTGTGCCATTTCGCCTGACTCATAATATGGTT
AATGGAATGGGTCCTATGGGAACAGAGGGTCTTTTTCGAAGAGCATGTGAAGTTACAATGAGGCTGATGC
GTGATCAGCGAGAGCCTTTAATGAGTGTCTTAAAGACTTTTCTACATGATCCTCTTGTGGAATGGAGTAA
ACCAGTGAAAGGGCATTCCAAAGCGCCACTGAATGAAACTGGAGAAGTTGTCAATGAAAAGGCCAAGACC
CATGTTCTTGACATTGAGCAGCGACTACAAGGTGTAATCAAGACTCGAAATAGAGTGACAGGACTGCCGT
TATCTATTGAAGGACATGTGCATTACCTTATACAGGAAGCTACTGATGAAAACTTACTATGCCAGATGTA
TCTTGGTTGGACTCCATATATGTGA
(SEQ ID NO: 5; NCBI Accession No. NM_001184.3)

### Kinase Inhibitors

In one aspect the invention provides an ataxia telangiectasia mutated (ATM) kinase inhibitor or an ataxia telangiectasia and Rad3-related protein (ATR) inhibitor for use in haematopoietic stem and/or progenitor cell gene therapy.

The invention also provides methods for identifying agents that are capable of acting as ATM kinase or ATR inhibitors and agents that are identified by such methods.

The activity of ATM kinase and ATR may be analysed directly, for example by analysing the enzymatic activity of the ATM kinase or ATR in vitro.

The ability of a candidate agent to inhibit (e.g. reduce the activity) ATM kinase or ATR may be expressed in terms of an IC50 value, which is the concentration of an agent that is required to give rise to a 50% reduction in the activity of the kinase. Preferably, the inhibitors of the invention have an IC50 value for inhibition (e.g. of ATM kinase or ATR) of less than 100 μM, more preferably less than 10 μM, for example less than 1 μM, less than 100 nM or less than 10 nM (e.g. KU-55933 has an IC50 value of about 13 nM for ATM kinase).

A number of techniques are known in the art for measuring kinase activity. Preferably, the kinase activity assays are carried out on a kinase (e.g. ATM kinase or ATR) that has been isolated from a cell. The kinase may have been expressed using recombinant techniques, and preferably has been purified. For example, kinase activity may be determined by monitoring the incorporation of radiolabelled phosphate from [γ-³²P]-labelled ATP into a substrate. Such assay techniques are described in, for example, Hastie et al. (Hastie, C. J. et al. (2006) Nat. Protocols 1:968-971).

Preferably, the inhibitors are of low toxicity for mammals, such as humans, and in particular are of low toxicity for haematopoietic stem and/or progenitor cells.

A candidate inhibitor may be further analysed for its ability to increase cell survival and/or engraftment using a method as disclosed herein.

Preferably, the inhibitor is a transient inhibitor (e.g. has an inhibitory action lasting less than about 1, 2, 3, 4, 5, 6, 7 or 14 days).

Preferably, the inhibitor is a pharmacological inhibitor.

KU-55933

In a preferred embodiment, the inhibitor is KU-55933 or a derivative thereof.

KU-55933 (CAS No. 587871-26-9) is a selective, competitive ATM kinase inhibitor having the following structure:

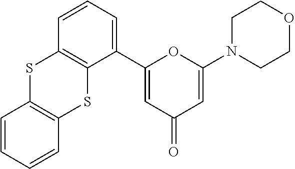 [SMILES: O=c1cc(-c2cccc3c2Sc2ccccc2S3)oc(N2CCOCC2)c1]

Solutions of KU-55933 for use in the invention may be prepared using routine methods known in the art, for example KU-55933 is known to be soluble in DMSO and ethanol.

The concentration at which KU-55933 or a derivative thereof is applied to a population of haematopoietic stem and/or progenitor cells may be adjusted for different vector systems to optimise cell survival (e.g. during in vitro or ex vivo culture) and/or engraftment.

The invention encompasses the use of KU-55933 and derivatives of KU-55933. The KU-55933 derivatives of the invention are those which increase the survival (e.g. during in vitro or ex vivo culture) and/or engraftment of haematopoietic stem and/or progenitor cells, in particular cells transduced by a viral vector.

KU-55933 derivatives of the invention may have been developed, for example, for increased solubility, increased stability and/or reduced toxicity.

KU-55933 derivatives of the invention are preferably of low toxicity for mammals, in particular humans. Preferably, KU-55933 derivatives of the invention are of low toxicity for haematopoietic stem and/or progenitor cells.

Suitable KU-55933 derivatives may be identified using methods known in the art for determining cell survival in culture and/or engraftment. Examples of such methods have been described above. The method employed is preferably one which is amenable to automation and/or high throughput screening of candidate KU-55933 derivatives. The candidate KU-55933 derivatives may form part of a library of KU-55933 derivatives.

### Additional Inhibitors

Further kinase inhibitors that may be used in the invention include:
KU-60019, which is an improved analogue of KU-55933 and has an IC50 of 6.3 nM for ATM kinase in cell-free assays. KU-60019 has the structure:

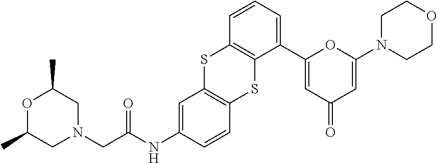 [SMILES: C[C@@H]1CN(CC(=O)Nc2ccc3c(c2)Sc2cccc(-c4cc(=O)cc(N5CCOCC5)o4)c2S3)C[C@H](C)O1]

BEZ235 (NVP-BEZ235, Dactolisib), which is a dual ATP-competitive PI3K and mTOR inhibitor for p110α/γ/δ/β and mTOR (p70S6K), and inhibits ATR with an IC50 of about 21 nM in 3T3TopBP1-ER cells. BEZ235 has the structure:

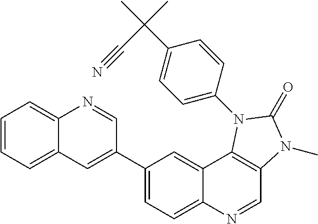 [SMILES: Cn1c(=O)n(-c2ccc(C(C)(C)C#N)cc2)c2c3cc(-c4cnc5ccccc5c4)ccc3ncc21]

Wortmannin, which has the structure:

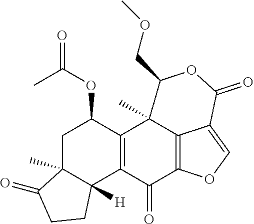 [SMILES: COC[C@H]1OC(=O)c2coc3c2[C@@]1(C)C1=C(C3=O)[C@@H]2CCC(=O)[C@@]2(C)C[C@H]1OC(C)=O]

CP-466722, which is a potent and reversible ATM kinase inhibitor, but does not affect ATR. CP-466722 has the structure:

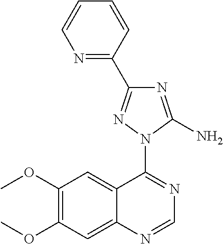 [SMILES: COc1cc2ncnc(-n3nc(-c4ccccn4)nc3N)c2cc1OC]

Torin 2, which ATM kinase and ATR with EC50 values of 28 nM and 35 nM, respectively. Torin 2 has the structure:

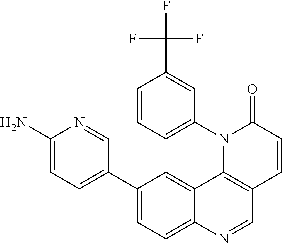 [SMILES: Nc1ccc(-c2ccc3ncc4ccc(=O)n(-c5cccc(C(F)(F)F)c5)c4c3c2)cn1]

CGK 733 (CAS No. 905973-89-9), which is a potent and selective inhibitor of ATM kinase and ATR with IC50 values of about 200 nM. CGK 733 has the structure:

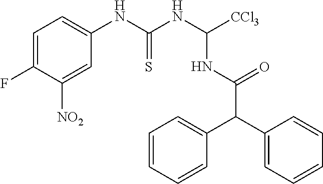

KU-559403 (Weber et al. (2015) Pharmacology & Therapeutics 149:124-138). KU-559403 has the structure:

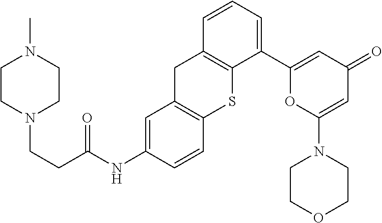 [SMILES: CN1CCN(CCC(=O)Nc2ccc3c(c2)Cc2cccc(-c4cc(=O)cc(N5CCOCC5)o4)c2S3)CC1]

AZD6738, which has the structure:

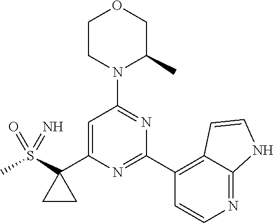 [SMILES: C[C@@H]1COCCN1c1cc(C2([S@](C)(=N)=O)CC2)nc(-c2ccnc3[nH]ccc23)n1]

Derivatives of these inhibitors, possessing characteristics as described for the KU-55933 derivatives, may also be used in the invention, and may be identified using analogous methods to those described for the KU-55933 derivatives.

siRNAs, shRNAs, miRNAs and antisense DNAs/RNAs

Inhibition (e.g. of the kinase) may be achieved using post-transcriptional gene silencing (PTGS). Post-transcriptional gene silencing mediated by double-stranded RNA (dsRNA) is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi) (Ralph et al. (2005) Nat. Medicine 11:429-433). The mechanism of RNAi involves the processing of long dsRNAs into duplexes of about 21-25 nucleotide (nt) RNAs. These products are called small interfering or silencing RNAs (siRNAs) which are the sequence-specific mediators of mRNA degradation. In differentiated mammalian cells, dsRNA >30 bp has been found to activate the interferon response leading to shut-down of protein synthesis and non-specific mRNA degradation (Stark et al. (1998) Ann. Rev. Biochem. 67:227-64). However, this response can be bypassed by using 21 nt siRNA duplexes (Elbashir et al. (2001) EMBO J. 20:6877-88; Hutvagner et al. (2001) Science 293:834-8) allowing gene function to be analysed in cultured mammalian cells.

shRNAs consist of short inverted RNA repeats separated by a small loop sequence. These are rapidly processed by the cellular machinery into 19-22 nt siRNAs, thereby suppressing the target gene expression.

Micro-RNAs (miRNAs) are small (22-25 nucleotides in length) noncoding RNAs that can effectively reduce the translation of target mRNAs by binding to their 3′ untranslated region (UTR). Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ˜70 nt precursor, which is post-transcriptionally processed into a mature ˜21 nt form. Both let-7 and lin-4 are transcribed as hairpin RNA precursors which are processed to their mature forms by Dicer enzyme.

The antisense concept is to selectively bind short, possibly modified, DNA or RNA molecules to messenger RNA in cells and prevent the synthesis of the encoded protein.

Methods for the design of siRNAs, shRNAs, miRNAs and antisense DNAs/RNAs to modulate the expression of a target protein, and methods for the delivery of these agents to a cell of interest are well known in the art.

### P53 Dominant Negative Peptide

The term “p53 dominant negative peptide”, as used herein, may refer to a peptide which inhibits the function of wild-type p53 when present in the same cell, for example a p53 dominant negative peptide may reduce or prevent p53 signalling.

In one embodiment, the p53 dominant negative peptide comprises or consists of GSE56 (Ossovskaya V. S. et al. (1996) Proc Natl Acad Sci USA 93:10309-10314).

GSE56 may have the amino acid sequence:

(SEQ ID NO: 6)
CPGRDRRTEEENFRKKEEHCPELPPGSAKRALPTSTSSSPQQKKKPLDGE
YFTLKIRGRERFEMFRELNEALELKDARAAEESGDSRAHSSYPK

In one embodiment, the p53 dominant negative peptide is a variant of GSE56 comprising 1, 2, 3, 4, 5, 6, 7, 8, 9 or 10 amino acid substitutions, additions or deletions, while retaining the activity of GSE56, for example in reducing or preventing p53 signalling.

In one embodiment, the p53 dominant negative peptide comprises an amino acid sequence having at least 70%, 75%, 80%, 85%, 90%, 95%, 96%, 97%, 98% or 99% identity to SEQ ID NO: 6. Preferably, reference to a sequence which has a percent identity to any one of the SEQ ID NOs detailed herein refers to a sequence which has the stated percent identity over the entire length of the SEQ ID NO referred to.

The p53 dominant negative peptide, for example GSE56, may be expressed in a cell using a viral vector (e.g. a retroviral, lentiviral or AAV vector) comprising a nucleotide sequence encoding the peptide.

### Haematopoietic Stem and Progenitor Cells

A stem cell is able to differentiate into many cell types. A cell that is able to differentiate into all cell types is known as totipotent. In mammals, only the zygote and early embryonic cells are totipotent. Stem cells are found in most, if not all, multicellular organisms. They are characterised by the ability to renew themselves through mitotic cell division and differentiate into a diverse range of specialised cell types. The two broad types of mammalian stem cells are embryonic stem cells that are isolated from the inner cell mass of blastocysts, and adult stem cells that are found in adult tissues. In a developing embryo, stem cells can differentiate into all of the specialised embryonic tissues. In adult organisms, stem cells and progenitor cells act as a repair system for the body, replenishing specialised cells, but also maintaining the normal turnover of regenerative organs, such as blood, skin or intestinal tissues.

Haematopoietic stem cells (HSCs) are multipotent stem cells that may be found, for example, in peripheral blood, bone marrow and umbilical cord blood. HSCs are capable of self-renewal and differentiation into any blood cell lineage. They are capable of recolonising the entire immune system, and the erythroid and myeloid lineages in all the haematopoietic tissues (such as bone marrow, spleen and thymus). They provide for life-long production of all lineages of haematopoietic cells.

Haematopoietic progenitor cells have the capacity to differentiate into a specific type of cell. In contrast to stem cells however, they are already far more specific: they are pushed to differentiate into their “target” cell. A difference between stem cells and progenitor cells is that stem cells can replicate indefinitely, whereas progenitor cells can only divide a limited number of times. Haematopoietic progenitor cells can be rigorously distinguished from HSCs only by functional in vivo assay (i.e. transplantation and demonstration of whether they can give rise to all blood lineages over prolonged time periods).

The haematopoietic stem and progenitor cells of the invention comprise the CD34 cell surface marker (denoted as CD34+).

Haematopoietic Stem and/or Progenitor Cell (HSPC) Sources

A population of haematopoietic stem and/or progenitor cells (HSPCs) may be obtained from a tissue sample.

For example, a population of haematopoietic stem and/or progenitor cells may be obtained from peripheral blood (e.g. adult and foetal peripheral blood), umbilical cord blood, bone marrow, liver or spleen. Preferably, these cells are obtained from peripheral blood or bone marrow. They may be obtained after mobilisation of the cells in vivo by means of growth factor treatment.

Mobilisation may be carried out using, for example, G-CSF, plerixaphor or combinations thereof. Other agents, such as NSAIDs and dipeptidyl peptidase inhibitors, may also be useful as mobilising agents.

With the availability of the stem cell growth factors GM-CSF and G-CSF, most haematopoietic stem cell transplantation procedures are now performed using stem cells collected from the peripheral blood, rather than from the bone marrow. Collecting peripheral blood stem cells provides a bigger graft, does not require that the donor be subjected to general anaesthesia to collect the graft, results in a shorter time to engraftment and may provide for a lower long-term relapse rate.

Bone marrow may be collected by standard aspiration methods (either steady-state or after mobilisation), or by using next-generation harvesting tools (e.g. Marrow Miner).

In addition, HSPCs may also be derived from induced pluripotent stem cells.

### Hsc Characteristics

HSCs are typically of low forward scatter and side scatter profile by flow cytometric procedures. Some are metabolically quiescent, as demonstrated by Rhodamine labelling which allows determination of mitochondrial activity. HSCs may comprise certain cell surface markers such as CD34, CD45, CD133, CD90 and CD49f. They may also be defined as cells lacking the expression of the CD38 and CD45RA cell surface markers. However, expression of some of these markers is dependent upon the developmental stage and tissue-specific context of the HSC. Some HSCs called “side population cells” exclude the Hoechst 33342 dye as detected by flow cytometry. Thus, HSCs have descriptive characteristics that allow for their identification and isolation.

### Negative Markers

CD38 is the most established and useful single negative marker for human HSCs.

Human HSCs may also be negative for lineage markers such as CD2, CD3, CD14, CD16, CD19, CD20, CD24, CD36, CD56, CD66b, CD271 and CD45RA. However, these markers may need to be used in combination for HSC enrichment.

By “negative marker” it is to be understood that human HSCs lack the expression of these markers.

### Positive Markers

CD34 and CD133 are the most useful positive markers for HSCs.

Some HSCs are also positive for lineage markers such as CD90, CD49f and CD93. However, these markers may need to be used in combination for HSC enrichment.

By “positive marker” it is to be understood that human HSCs express these markers.

### Differentiated Cells

A differentiated cell is a cell which has become more specialised in comparison to a stem cell or progenitor cell. Differentiation occurs during the development of a multicellular organism as the organism changes from a single zygote to a complex system of tissues and cell types. Differentiation is also a common process in adults: adult stem cells divide and create fully-differentiated daughter cells during tissue repair and normal cell turnover. Differentiation dramatically changes a cell's size, shape, membrane potential, metabolic activity and responsiveness to signals. These changes are largely due to highly-controlled modifications in gene expression. In other words, a differentiated cell is a cell which has specific structures and performs certain functions due to a developmental process which involves the activation and deactivation of specific genes. Here, a differentiated cell includes differentiated cells of the haematopoietic lineage such as monocytes, macrophages, neutrophils, basophils, eosinophils, erythrocytes, megakaryocytes/platelets, dendritic cells, T-cells, B-cells and NK-cells. For example, differentiated cells of the haematopoietic lineage can be distinguished from stem cells and progenitor cells by detection of cell surface molecules which are not expressed or are expressed to a lesser degree on undifferentiated cells. Examples of suitable human lineage markers include CD33, CD13, CD14, CD15 (myeloid), CD19, CD20, CD22, CD79a (B), CD36, CD71, CD235a (erythroid), CD2, CD3, CD4, CD8 (T) and CD56 (NK).

### Isolation and Enrichment of Populations of Cells

By “isolated population” of cells it is to be understood that the population of cells has been previously removed from the body. An isolated population of cells may be cultured and manipulated ex vivo or in vitro using standard techniques known in the art. An isolated population of cells may later be reintroduced into a subject. Said subject may be the same subject from which the cells were originally isolated or a different subject.

A population of cells may be purified selectively for cells that exhibit a specific phenotype or characteristic, and from other cells which do not exhibit that phenotype or characteristic, or exhibit it to a lesser degree. For example, a population of cells that expresses a specific marker (such as CD34) may be purified from a starting population of cells. Alternatively, or in addition, a population of cells that does not express another marker (such as CD38) may be purified.

By “enriching” a population of cells for a certain type of cells it is to be understood that the concentration of that type of cells is increased within the population. The concentration of other types of cells may be concomitantly reduced.

Purification or enrichment may result in the population of cells being substantially pure of other types of cell.

Purifying or enriching for a population of cells expressing a specific marker (e.g. CD34 or CD38) may be achieved by using an agent that binds to that marker, preferably substantially specifically to that marker.

An agent that binds to a cellular marker may be an antibody, for example an anti-CD34 or anti-CD38 antibody.

The term “antibody” refers to complete antibodies or antibody fragments capable of binding to a selected target, and including Fv, ScFv, F(ab′) and F(ab′)₂, monoclonal and polyclonal antibodies, engineered antibodies including chimeric, CDR-grafted and humanised antibodies, and artificially selected antibodies produced using phage display or alternative techniques.

In addition, alternatives to classical antibodies may also be used in the invention, for example “avibodies”, “avimers”, “anticalins”, “nanobodies” and “DARPins”.

The agents that bind to specific markers may be labelled so as to be identifiable using any of a number of techniques known in the art. The agent may be inherently labelled, or may be modified by conjugating a label thereto. By “conjugating” it is to be understood that the agent and label are operably linked. This means that the agent and label are linked together in a manner which enables both to carry out their function (e.g. binding to a marker, allowing fluorescent identification, or allowing separation when placed in a magnetic field) substantially unhindered. Suitable methods of conjugation are well known in the art and would be readily identifiable by the skilled person.

A label may allow, for example, the labelled agent and any cell to which it is bound to be purified from its environment (e.g. the agent may be labelled with a magnetic bead or an affinity tag, such as avidin), detected or both. Detectable markers suitable for use as a label include fluorophores (e.g. green, cherry, cyan and orange fluorescent proteins) and peptide tags (e.g. His tags, Myc tags, FLAG tags and HA tags).

A number of techniques for separating a population of cells expressing a specific marker are known in the art. These include magnetic bead-based separation technologies (e.g. closed-circuit magnetic bead-based separation), flow cytometry, fluorescence-activated cell sorting (FACS), affinity tag purification (e.g. using affinity columns or beads, such as biotin columns to separate avidin-labelled agents) and microscopy-based techniques.

It may also be possible to perform the separation using a combination of different techniques, such as a magnetic bead-based separation step followed by sorting of the resulting population of cells for one or more additional (positive or negative) markers by flow cytometry.

Clinical grade separation may be performed, for example, using the CliniMACS® system (Miltenyi). This is an example of a closed-circuit magnetic bead-based separation technology.

It is also envisaged that dye exclusion properties (e.g. side population or rhodamine labelling) or enzymatic activity (e.g. ALDH activity) may be used to enrich for HSCs.

### Vectors

A vector is a tool that allows or facilitates the transfer of an entity from one environment to another. The vectors used to transduce haematopoietic stem and/or progenitor cells in the invention are viral vectors.

Preferably, the viral vector is in the form of a viral vector particle.

The viral vector may be, for example, a retroviral, lentiviral, adeno-associated viral (AAV) or adenoviral vector. Preferably, the viral vector is a lentiviral or AAV vector, more preferably a lentiviral vector. Preferably, the retroviral vector is not a γ-retroviral vector.

By “vector derived from” a certain type of virus, it is to be understood that the vector comprises at least one component part derivable from that type of virus.

### Retroviral and Lentiviral Vectors

A retroviral vector may be derived from or may be derivable from any suitable retrovirus. A large number of different retroviruses have been identified. Examples include murine leukaemia virus (MLV), human T-cell leukaemia virus (HTLV), mouse mammary tumour virus (MMTV), Rous sarcoma virus (RSV), Fujinami sarcoma virus (FuSV), Moloney murine leukaemia virus (Mo-MLV), FBR murine osteosarcoma virus (FBR MSV), Moloney murine sarcoma virus (Mo-MSV), Abelson murine leukaemia virus (A-MLV), avian myelocytomatosis virus-29 (MC29) and avian erythroblastosis virus (AEV). A detailed list of retroviruses may be found in Coffin, J. M. et al. (1997) Retroviruses, Cold Spring Harbour Laboratory Press, 758-63.

Retroviruses may be broadly divided into two categories, “simple” and “complex”. Retroviruses may be even further divided into seven groups. Five of these groups represent retroviruses with oncogenic potential. The remaining two groups are the lentiviruses and the spumaviruses. A review of these retroviruses is presented in Coffin, J. M. et al. (1997) Retroviruses, Cold Spring Harbour Laboratory Press, 758-63.

The basic structure of retrovirus and lentivirus genomes share many common features such as a 5′ LTR and a 3′ LTR. Between or within these are located a packaging signal to enable the genome to be packaged, a primer binding site, integration sites to enable integration into a host cell genome, and gag, pol and env genes encoding the packaging components—these are polypeptides required for the assembly of viral particles. Lentiviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration and transcription. LTRs also serve as enhancer-promoter sequences and can control the expression of the viral genes.

The LTRs themselves are identical sequences that can be divided into three elements: U3, R and U5. U3 is derived from the sequence unique to the 3′ end of the RNA. R is derived from a sequence repeated at both ends of the RNA. U5 is derived from the sequence unique to the 5′ end of the RNA. The sizes of the three elements can vary considerably among different retroviruses.

In a defective retroviral vector genome gag, pol and env may be absent or not functional.

In a typical retroviral vector, at least part of one or more protein coding regions essential for replication may be removed from the virus. This makes the viral vector replication-defective. Portions of the viral genome may also be replaced by a library encoding candidate modulating moieties operably linked to a regulatory control region and a reporter moiety in the vector genome in order to generate a vector comprising candidate modulating moieties which is capable of transducing a target host cell and/or integrating its genome into a host genome.

Lentivirus vectors are part of the larger group of retroviral vectors. A detailed list of lentiviruses may be found in Coffin, J. M. et al. (1997) Retroviruses, Cold Spring Harbour Laboratory Press, 758-63. In brief, lentiviruses can be divided into primate and non-primate groups. Examples of primate lentiviruses include but are not limited to human immunodeficiency virus (HIV), the causative agent of human acquired immunodeficiency syndrome (AIDS); and simian immunodeficiency virus (SIV). Examples of non-primate lentiviruses include the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

The lentivirus family differs from retroviruses in that lentiviruses have the capability to infect both dividing and non-dividing cells (Lewis, P et al. (1992) EMBO J. 11:3053-8; Lewis, P. F. et al. (1994) J. Virol. 68:510-6). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects cells, expresses genes or is replicated.

The lentiviral vector may be a “primate” vector. The lentiviral vector may be a “non-primate” vector (i.e. derived from a virus which does not primarily infect primates, especially humans).

Examples of non-primate lentiviruses may be any member of the family of lentiviridae which does not naturally infect a primate.

As examples of lentivirus-based vectors, HIV-1- and HIV-2-based vectors are described below.

The HIV-1 vector contains cis-acting elements that are also found in simple retroviruses. It has been shown that sequences that extend into the gag open reading frame are important for packaging of HIV-1. Therefore, HIV-1 vectors often contain the relevant portion of gag in which the translational initiation codon has been mutated. In addition, most HIV-1 vectors also contain a portion of the env gene that includes the RRE. Rev binds to RRE, which permits the transport of full-length or singly spliced mRNAs from the nucleus to the cytoplasm. In the absence of Rev and/or RRE, full-length HIV-1 RNAs accumulate in the nucleus. Alternatively, a constitutive transport element from certain simple retroviruses such as Mason-Pfizer monkey virus can be used to relieve the requirement for Rev and RRE. Efficient transcription from the HIV-1 LTR promoter requires the viral protein Tat.

Most HIV-2-based vectors are structurally very similar to HIV-1 vectors. Similar to HIV-1-based vectors, HIV-2 vectors also require RRE for efficient transport of the full-length or singly spliced viral RNAs.

In one system, the vector and helper constructs are from two different viruses, and the reduced nucleotide homology may decrease the probability of recombination. In addition to vectors based on the primate lentiviruses, vectors based on FIV have also been developed as an alternative to vectors derived from the pathogenic HIV-1 genome. The structures of these vectors are also similar to the HIV-1 based vectors.

Preferably, the viral vector used in the present invention has a minimal viral genome.

By “minimal viral genome” it is to be understood that the viral vector has been manipulated so as to remove the non-essential elements and to retain the essential elements in order to provide the required functionality to infect, transduce and deliver a nucleotide sequence of interest to a target host cell. Further details of this strategy can be found in WO 1998/017815.

Preferably, the plasmid vector used to produce the viral genome within a host cell/packaging cell will have sufficient lentiviral genetic information to allow packaging of an RNA genome, in the presence of packaging components, into a viral particle which is capable of infecting a target cell, but is incapable of independent replication to produce infectious viral particles within the final target cell. Preferably, the vector lacks a functional gag-pol and/or env gene and/or other genes essential for replication.

However, the plasmid vector used to produce the viral genome within a host cell/packaging cell will also include transcriptional regulatory control sequences operably linked to the lentiviral genome to direct transcription of the genome in a host cell/packaging cell. These regulatory sequences may be the natural sequences associated with the transcribed viral sequence (i.e. the 5′ U3 region), or they may be a heterologous promoter, such as another viral promoter (e.g. the CMV promoter).

The vectors may be self-inactivating (SIN) vectors in which the viral enhancer and promoter sequences have been deleted. SIN vectors can be generated and transduce non-dividing cells in vivo with an efficacy similar to that of wild-type vectors. The transcriptional inactivation of the long terminal repeat (LTR) in the SIN provirus should prevent mobilisation by replication-competent virus. This should also enable the regulated expression of genes from internal promoters by eliminating any cis-acting effects of the LTR.

The vectors may be integration-defective. Integration defective lentiviral vectors (IDLVs) can be produced, for example, either by packaging the vector with catalytically inactive integrase (such as an HIV integrase bearing the D64V mutation in the catalytic site; Naldini, L. et al. (1996) Science 272:263-7; Naldini, L. et al. (1996) Proc. Natl. Acad. Sci. USA 93:11382-8; Leavitt, A. D. et al. (1996) J. Virol. 70:721-8) or by modifying or deleting essential att sequences from the vector LTR (Nightingale, S. J. et al. (2006) Mol. Ther. 13:1121-32), or by a combination of the above.

### Adeno-Associated Viral (AAV) Vectors

Adeno-associated virus (AAV) is an attractive vector system for use in the invention as it has a high frequency of integration and it can infect non-dividing cells. This makes it useful for delivery of genes into mammalian cells in tissue culture.

AAV has a broad host range for infectivity. Details concerning the generation and use of AAV vectors are described in U.S. Pat. Nos. 5,139,941 and 4,797,368.

Recombinant AAV vectors have been used successfully for in vitro and in vivo transduction of marker genes and genes involved in human diseases.

### Adenoviral Vectors

The adenovirus is a double-stranded, linear DNA virus that does not go through an RNA intermediate. There are over 50 different human serotypes of adenovirus divided into 6 subgroups based on the genetic sequence homology. The natural targets of adenovirus are the respiratory and gastrointestinal epithelia, generally giving rise to only mild symptoms. Serotypes 2 and 5 (with 95% sequence homology) are most commonly used in adenoviral vector systems and are normally associated with upper respiratory tract infections in the young.

Adenoviruses have been used as vectors for gene therapy and for expression of heterologous genes. The large (36 kb) genome can accommodate up to 8 kb of foreign insert DNA and is able to replicate efficiently in complementing cell lines to produce very high titres of up to 1012. Adenovirus is thus one of the best systems to study the expression of genes in primary non-replicative cells.

The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, they function episomally (independently from the host genome) as a linear genome in the host nucleus. Hence the use of recombinant adenovirus alleviates the problems associated with random integration into the host genome.

### Nucleotide of Interest

The vector of the invention preferably comprises a nucleotide of interest (NOI).

Preferably the nucleotide of interest gives rise to a therapeutic effect.

Suitable NOIs include, but are not limited to, sequences encoding enzymes, cytokines, chemokines, hormones, antibodies, anti-oxidant molecules, engineered immunoglobulin-like molecules, single chain antibodies, fusion proteins, immune co-stimulatory molecules, immunomodulatory molecules, anti-sense RNA, microRNA, shRNA, siRNA, ribozymes, miRNA target sequences, a transdomain negative mutant of a target protein, toxins, conditional toxins, antigens, tumour suppressor proteins, growth factors, transcription factors, membrane proteins, surface receptors, anti-cancer molecules, vasoactive proteins and peptides, anti-viral proteins and ribozymes, and derivatives thereof (such as derivatives with an associated reporter group). The NOIs may also encode pro-drug activating enzymes.

An example of a NOI is the beta-globin chain which may be used for gene therapy of thalassemia/sickle cell disease.

NOIs also include those useful for the treatment of other diseases requiring non-urgent/elective gene correction in the myeloid lineage such as: chronic granulomatous disease (CGD, e.g. the gp91phox transgene), leukocyte adhesion defects, other phagocyte disorders in patients without ongoing severe infections and inherited bone marrow failure syndromes (e.g. Fanconi anaemia), as well as primary immunodeficiencies (SCIDs).

NOIs also include those useful in the treatment of lysosomal storage disorders and immunodeficiencies.

### Pharmaceutical Composition

The cells of the invention may be formulated for administration to subjects with a pharmaceutically acceptable carrier, diluent or excipient. Suitable carriers and diluents include isotonic saline solutions, for example phosphate-buffered saline, and potentially contain human serum albumin.

Handling of cell therapy products is preferably performed in compliance with FACT-JACIE International Standards for cellular therapy.

Haematopoietic Stem and/or Progenitor Cell Transplantation

The invention provides a population of haematopoietic stem and/or progenitor cells prepared according to a method of the invention for use in therapy, for example for use in gene therapy.

The use may be as part of a haematopoietic stem and/or progenitor cell transplantation procedure.

Haematopoietic stem cell transplantation (HSCT) is the transplantation of blood stem cells derived from the bone marrow (in this case known as bone marrow transplantation) or blood. Stem cell transplantation is a medical procedure in the fields of haematology and oncology, most often performed for people with diseases of the blood or bone marrow, or certain types of cancer.

Many recipients of HSCTs are multiple myeloma or leukaemia patients who would not benefit from prolonged treatment with, or are already resistant to, chemotherapy. Candidates for HSCTs include paediatric cases where the patient has an inborn defect such as severe combined immunodeficiency or congenital neutropenia with defective stem cells, and also children or adults with aplastic anaemia who have lost their stem cells after birth. Other conditions treated with stem cell transplants include sickle-cell disease, myelodysplastic syndrome, neuroblastoma, lymphoma, Ewing's Sarcoma, Desmoplastic small round cell tumour and Hodgkin's disease. More recently non-myeloablative, or so-called “mini transplant”, procedures have been developed that require smaller doses of preparative chemotherapy and radiation. This has allowed HSCT to be conducted in the elderly and other patients who would otherwise be considered too weak to withstand a conventional treatment regimen.

In one embodiment, a population of haematopoietic stem and/or progenitor cells prepared according to a method of the invention is administered as part of an autologous stem cell transplant procedure.

In another embodiment, a population of haematopoietic stem and/or progenitor cells prepared according to a method of the invention is administered as part of an allogeneic stem cell transplant procedure.

By “autologous stem cell transplant procedure” it is to be understood that the starting population of cells (which are then transduced according to a method of the invention) is obtained from the same subject as that to which the transduced cell population is administered. Autologous transplant procedures are advantageous as they avoid problems associated with immunological incompatibility and are available to subjects irrespective of the availability of a genetically matched donor.

By “allogeneic stem cell transplant procedure” it is to be understood that the starting population of cells (which are then transduced according to a method of the invention) is obtained from a different subject as that to which the transduced cell population is administered. Preferably, the donor will be genetically matched to the subject to which the cells are administered to minimise the risk of immunological incompatibility.

Suitable doses of transduced cell populations are such as to be therapeutically and/or prophylactically effective. The dose to be administered may depend on the subject and condition to be treated, and may be readily determined by a skilled person.

Haematopoietic progenitor cells provide short term engraftment. Accordingly, gene therapy by administering transduced haematopoietic progenitor cells would provide a non-permanent effect in the subject. For example, the effect may be limited to 1-6 months following administration of the transduced haematopoietic progenitor cells. An advantage of this approach would be better safety and tolerability, due to the self-limited nature of the therapeutic intervention.

Such haematopoietic progenitor cell gene therapy may be suited to treatment of acquired disorders, for example cancer, where time-limited expression of a (potentially toxic) anti-cancer nucleotide of interest may be sufficient to eradicate the disease.

The invention (e.g. the haematopoietic stem and/or progenitor cell gene therapy) may be, for example, useful in the treatment of a disease selected from the group consisting of mucopolysaccharidosis type I (MPS-1), chronic granulomatous disorder (CGD), Fanconi anaemia (FA), sickle cell disease, Pyruvate kinase deficiency (PKD), Leukocyte adhesion deficiency (LAD), metachromatic leukodystrophy (MLD), globoid cell leukodystrophy (GLD), GM₂ gangliosidosis, thalassemia and cancer.

The invention may also be, for example, useful in the treatment of mucopolysaccharidoses disorders and other lysosomal storage disorders.

In addition, or in the alternative, the invention may be useful in the treatment of the disorders listed in WO 1998/005635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the invention may be useful in the treatment of the disorders listed in WO 1998/007859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatics and thrombolytic activity (e.g. for treating haemophilia and stroke); anti-inflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the invention may be useful in the treatment of the disorders listed in WO 1998/009985. For ease of reference, part of that list is now provided: macrophage inhibitory and/or T cell inhibitory activity and thus, anti-inflammatory activity;

anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

### Kit

In another aspect, the invention provides a kit comprising an inhibitor and/or cell populations of the invention.

The inhibitor and/or cell populations may be provided in suitable containers.

The kit may also include instructions for use.

### Method of Treatment

It is to be appreciated that all references herein to treatment include curative, palliative and prophylactic treatment. The treatment of mammals, particularly humans, is preferred. Both human and veterinary treatments are within the scope of the invention.

### Administration

Although the agents for use in the invention (in particular, the populations of cells produced by a method of the invention) can be administered alone, they will generally be administered in admixture with a pharmaceutical carrier, excipient or diluent, particularly for human therapy.

### Dosage

The skilled person can readily determine an appropriate dose of one of the agents of the invention to administer to a subject without undue experimentation. Typically, a physician will determine the actual dosage which will be most suitable for an individual patient and it will depend on a variety of factors including the activity of the specific agent employed, the metabolic stability and length of action of that agent, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

### Subject

A “subject” refers to either a human or non-human animal.

Examples of non-human animals include vertebrates, for example mammals, such as non-human primates (particularly higher primates), dogs, rodents (e.g. mice, rats or guinea pigs), pigs and cats. The non-human animal may be a companion animal.

Preferably, the subject is a human.

### EXAMPLES

### Example 1

### Materials and Methods

### Viral Vectors

Third generation self-inactivating lentiviral vectors (LVs) and integrase-defective LV (IDLV) expressing GFP under the control of an internal PGK promoter (SINLV-GFP) stocks were prepared, concentrated and titered as described in Follenzi et al. and Lombardo et al. (Follenzi, A. et al. (2000) Nature Genetics 25:217-222; Lombardo, A. et al. (2007) Nature Biotechnology 25:1298-1306). Purified LV was obtained as described in Biffi et al. (Biffi, A. et al. (2013) Science 341:1233158). Retroviral Vectors were prepared and used as described in Montini et al. (Montini, E. et al. (2006) Nature Biotechnology 24:687-696). Bald vector, an entry-incompetent LV was produced omitting the Env-encoding plasmid during vector production. Empty LV, a genome-less lentiviral vector was produced omitting the SIN-LV PGK-GFP transfer vector during vector production. To produce the p21 overexpressing LV we replaced the Thymidine Kinase cDNA of a previously described bidirectional LV expression cassette (Amendola, M. et al. (2005) Nature Biotechnology 23:108-116), with the cDNA of the human p21 gene which is expressed under the control of the human PGK promoter. The GFP is expressed from the minimal CMV promoter in the opposite orientation. Inactivated purified LV were obtaining by heating (1 h at 56° C.) purified LV. AAV6-IL2RG-eGFP was as described in Wang et al. (Wang, J. et al. (2015) Nature Biotechnology 33:1256-1263) except that instead of the homology arms for CCR5 or AAVS1 locus, it contains the homology arms for the IL2RG locus, as described in Genovese et al. (Genovese et al. (2014) Nature 510:235-40).

### Cell Culture and Transduction

Human CD34+ hematopoietic stem and progenitor cells (HSPC) and CD4+ T cells were isolated through magnetic bead selection according to the manufacturer's instructions (Milteny) from umbilical cord blood (CB) collected upon informed consent from healthy volunteers according to the Institutional Ethical Committee approved protocol (TIGET01). Otherwise, CB and bone marrow (BM)-derived CD34+ were directly purchased from Lonza. Cells were purified and cultured as described in Petrillo et al. (Petrillo, C. et al. (2015) Molecular Therapy 23:352-362). K562 and Hela cell lines were plated in complete Iscove's modified Dulbecco's medium (Euroclone). HL-60 cells were grown and transduced in complete RPMI. HCT-116 cells were grown and transduced in complete Dulbecco's Modified Eagle Medium (DMEM). Murine Lin-cells were isolated through magnetic bead selection according to the manufacturer's instructions (Miltenyi) from bone marrow of euthanised C57/BL6 mice. For the clinical standard double-hit transduction protocol, cells were washed 16 h after the first vector exposure, left to recover in cytokine-supplemented medium for 10 h and re-exposed to the second hit of vector for another 16 h before transplantation. All animal procedures were performed according to protocols approved by the Animal Care and Use Committee of the Ospedale San Raffaele (IACUC 611) and according to Italian law. All cells were transduced at the indicated multiplicity of infection (MOI) as calculated by titration of vector stocks on 293T cells for Lentiviral and Retroviral vectors. Transductions with the AAV6 vectors were performed at an MOI of 10000 as calculated by titration of the vector preparation expressed in vector genome per mL (vg/mL). All the transductions were performed at 1×10⁶ cells/mL concentration. In the experiment with anti-retroviral inhibitors, the drugs were added together with the vector. Raltegravir and 3TC were used at 10 μM, and AZT at 25 μM. In the ATM inhibition experiment, single hit of the ATM inhibitor KU55933 (Selleck Chemicals) was added to the cells 2 hours before the transduction at 10 μM concentration. All cells were maintained in a 5% CO₂ humidified atmosphere at 37° C.

### Colony-Forming Cell (CFC) Assay and Transplantation of Human HSPC in NSG Mice

CFC assay cells were performed as described in Petrillo et al and Gentner et al. (Petrillo, C. et al. (2015) Molecular Therapy 23:352-362; Gentner, B. et al. (2010) Science Translational Medicine 2: 58ra84). After ten days, colonies were identified as erythroid or myeloid by morphological criteria, counted, plucked and pooled into sets of three to obtain DNA for quantification of vector content. (NSG) mice were purchased from Jackson laboratory. Human CB-derived CD34+ cells were pre-stimulated and transduced as indicated. Eight to ten week old NSG mice were sub-lethally irradiated (radiation dose: 200 cGy for mice weighing 18-25 g and of 220 cGy for mice above 25 g in weight) 24 hours prior to xenotransplantation. 8×10⁴ human CB-derived CD34+ cells were injected into the tail vein of primary NSG mice 24 hours after transduction. Peripheral blood was sampled at indicated times post-transplant and analysed as described in Petrillo et al. (Petrillo, C. et al. (2015) Molecular Therapy 23:352-362). At sacrifice, the cells from the spleen and BM isolated from the primary recipients were analysed by flow cytometry and the CD34+ cells were purified from the BM through positive magnetic bead selection on LD and MS columns (Miltenyi) according to the manufacturer's instructions. Purity was verified by FACS prior to pooling by condition and injection into secondary recipients. Between 9×10⁵ and 1×10⁶ CD34+ cells isolated from the primary hosts were injected into the tail vein of sub-lethally irradiated secondary NSG mice (8-10 weeks old). Peripheral blood was sampled at indicated times post-transplant and analysed as described above. At 14 weeks of age, all mice were sacrificed by CO₂ to analyse the BM and the spleen of the secondary mice as described above.

### Limiting Dilution Assay

Limiting dilution assays were performed as previously described (Lechman, E. R. et al. (2012) Cell Stem Cell 11:799-811; Petrillo, C. et al. (2015) Mol Ther 23:352-362) by transplanting into irradiated 8-week old NSG mice 4 different doses of CB-CD34+ cells counted prior to transduction (3000, 10000, 30000 or 90000) either for Bald and LV groups. Cells were injected 24 hours after transduction. A mouse was scored as positively engrafted if it had >0.1% engraftment in multiple lineages in the BM at the time of sacrifice (16 weeks). HSC frequency was estimated by linear regression analysis and Poisson statistics using publicly available ELDA (Extreme Limiting Dilution Analysis, http COLON SLASH SLASH bioinf.wehi.edu.au/software/elda/) software (Hu et al. (2009) J Immunol Methods 347:70-78).

### Homing Assay

For homing assays, 5×10⁵ CB-derived CD34+ cells (pre-treatment dose) were transplanted 24 h after transduction into irradiated 8-week old NSG mice that were sacrificed 16 h post-transplantation for analysis. The whole bone marrow was harvested from the lower leg of the mice. During the FACS analysis cell counts beads (Flow-Count Fluorospheres) by BECKMAN CULTER were added in each sample to estimate the absolute number of human CD34+ cells per sample. Cell counts for all in vivo experiments before and after treatment, prior to transplantation are provided in Table 9.

### RNA-Seq Data Generation and Analysis

Total RNA was extracted with the RNeasy Plus Micro kit (Qiagen) according to the manufacturer's instructions. RNA integrity was analysed with the Agilent 2100 Bioanalyzer (Agilent Technologies, Santa Clara, CA). Libraries, prepared from 100 ng of total RNA/sample with the Illumina TruSeq RNA Sample Prep kit v2 procedure, were quantified by the Qubit BR assay (Life Technologies, Illkirch, France) and the Agilent 2100 Bioanalyzer. Sequencing was performed on the Illumina HiSeq 2000 platform using SBS 2×100PE protocol. On average for each sample we obtained 30M reads. Each gene was then characterised by the total number of reads overlapping it. Normalisation and differential gene expression was performed with the Bioconductor of the limma package (Smyth, G. K. (2004) Statistical Applications in Genetics and Molecular Biology 3: Article 3). Pathway analysis was initially performed using EnrichrR platform (Chen, E. Y. et al. (2013) BMC Bioinformatics 14:128), most of the advanced network modelling was performed using Cytoscape.

### FACS and Flow Cytometry

Human HSPC fluorescence associated cell sorting was performed as described in Petrillo et al. (Petrillo, C. et al. (2015) Molecular Therapy 23:352-362). All cytometric analyses were performed using the FACS Canto III instrument and LSRFortessa instruments (BD Biosciences, San Jose, CA) and analysed with the FACS Express software (De Novo Software, Glendale, CA). GFP expression in transduced cells was measured 5-7 days post-transduction. To exclude dead cells from the analysis, cells were washed and resuspended in PBS containing 10 ng/ml 7-aminoactinomycin D (7-AAD, Sigma-Aldrich). The apoptosis assays were performed with the Annexin V Apoptosis Detection Kit I (BD Pharmigen) according to the manufacturer's instructions and 48 hours after transduction, if not otherwise indicated. The cell proliferation assay was performed with the Cell Proliferation Dye eFluor 670 (eBioscience) according to the manufacturer's instructions. To calibrate the cytometer, rainbow beads (Spherotech) were used to set the dye signal on the different days of the analysis. The cell cycle analysis was performed by Ki67 (BD Pharmigen) and Hoechst (Invitrogen) staining as described in Lechman et al. (Lechman, E. R. et al. (2012) Cell Stem Cell 11:799-811) 48 hours after the transduction. Antibodies are shown in Table 4.

### RNA, DNA and Proteins

Total RNA was extracted with the RNeasy Plus Micro kit or RNeasy Micro Kit (Qiagen) and Reverse transcription was performed using SuperScriptVILO cDNA Synthesis Kit (ThermoFisher Scientific) according to the manufacturers' instructions. Gene expression analysis was performed by Taqman probe (Thermo Fisher, Table 6) as described in Petrillo et al. (Petrillo, C. et al. (2015) Molecular Therapy 23:352-362), human HPRT1 or murine Hprt were used to normalise the total quantity of human or mouse cDNA input respectively. Table 6 shows the complete list of Taqman Probe reagents.

### Vector Copy Number (VCN)

For vector copy number (VCN), total DNA was extracted using a Maxwell 16 instrument (Promega) or Blood & Cell Culture DNA micro kit (Qiagen). Copy number of the integrated lentiviral vector was assessed as described in Lombardo et al., Petrillo et al. and Santoni de Sio et al. (Lombardo, A. et al. (2007) Nature Biotechnology 25:1298-1306; Petrillo, C. et al. (2015) Molecular Therapy 23:352-362; Santoni de Sio, F. R. et al. (2008) Stem Cells 26:2142-2152) or by digital droplet PCR (dd-PCR; BIO-RAD, California, USA) according to the manufacturers' instructions using hTERT gene as a normaliser. Copy number of the total lentiviral DNA (integrated and non-integrated) was analysed as described in Matrai et al. (Matrai, J. et al. (2011) Hepatology 53:1696-1707) at three days post-transduction. Copy number of the reverse transcribed retroviral vector genome (both integrated and non-integrated) was performed by dd-PCR discriminating it from plasmid carried over from the transient transfection using the following primers: RT-RV; AU3 sense: 5′-CGAGCTCAATAAAAGAGCCCAC-3′ (SEQ ID NO: 7), PBS antisense: 5′-GAGTCCTGCGTCGGAGAGAG-3′ (SEQ ID NO: 8). The amount of human DNA loaded in the reaction was quantified with a qPCR or ddPCR designed to amplify the hTERT gene as described in Lombardo et al. (Lombardo, A. et al. (2007) Nature Biotechnology 25:1298-1306). The 2LTR circle copy number was performed in dd-PCR with primers as described in Petrillo et al. (Petrillo, C. et al. (2015) Molecular Therapy 23:352-362). Table 7 shows a complete list of primers. Unless otherwise specified, copy numbers are expressed as amplicon copies per cell (diploid genome).

### Western Blot

Western Blot was performed as described in Petrillo et al. and Kajaste-Rudnitski et al. (Petrillo, C. et al. (2015) Molecular Therapy 23:352-362; Kajaste-Rudnitski, A. et al. (2006) The Journal of Biological Chemistry 281:4624-4637). Samples were subjected to SDS-PAGE on Bolt 4-12% Bis-Tris Plus gels (ThermoFisher, CAT #NW04120BOX) and transferred to PVDF membrane by electroblotting. Table 5 shows a complete list of antibodies.

### Statistical Analysis

Data is expressed as mean±standard error of the mean (SEM). Statistical tests were performed as indicated for each experiment. Significance was considered at p<0.05.

### Results

### Lentiviral Transduction Triggers DNA-Damage Responses in Human HSPCs

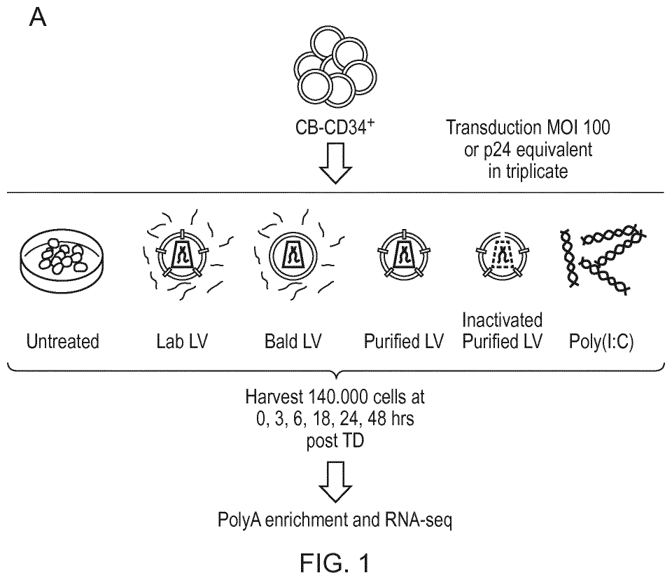

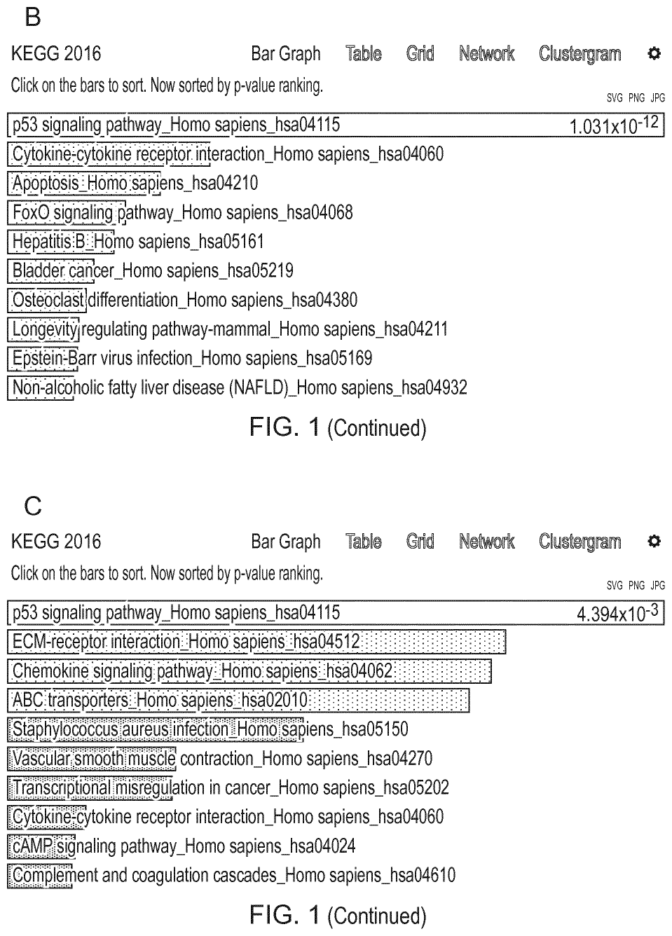

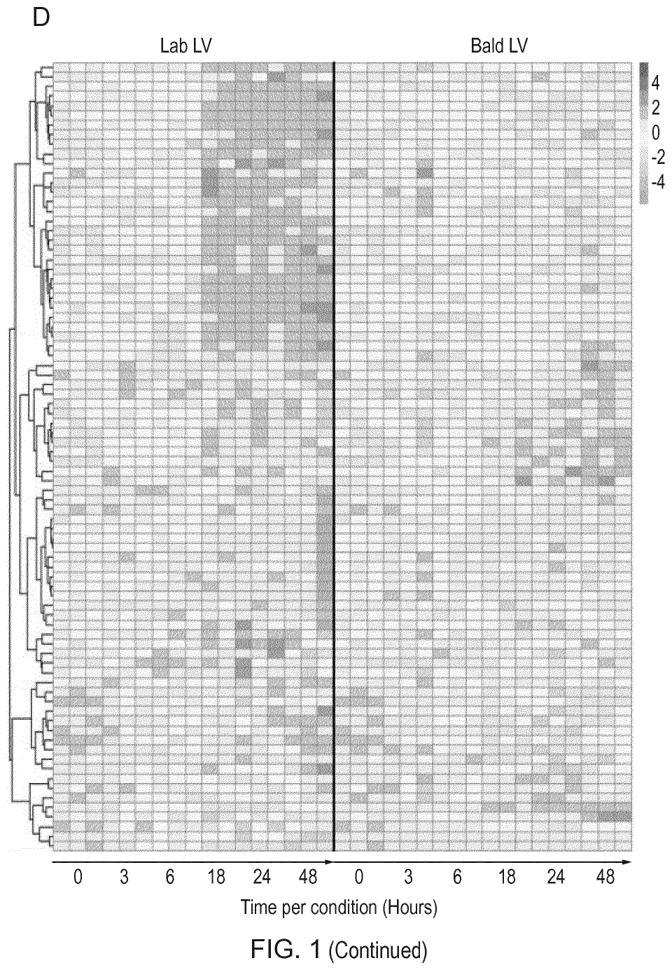

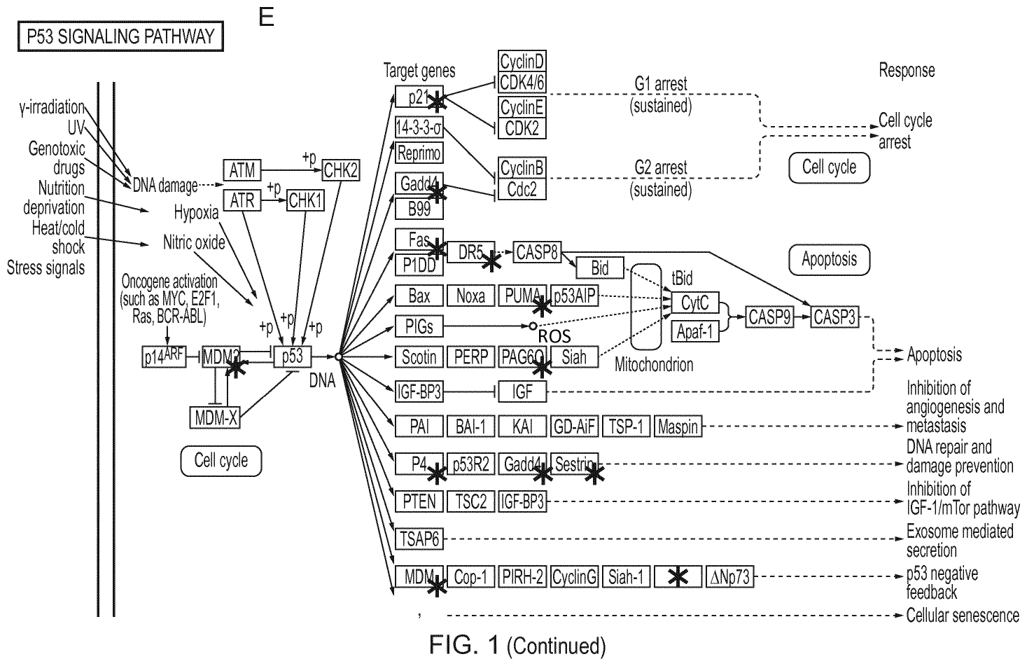

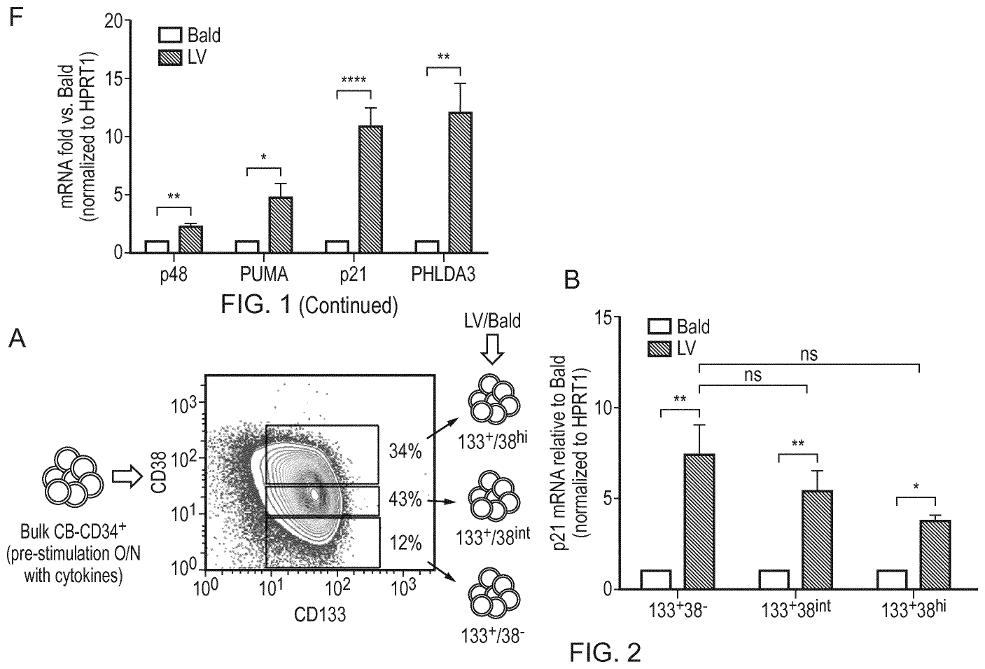

To extensively address the signalling potentially occurring upon exposure of human HSPCs to LVs, we performed RNA-Seq analysis in time-course on cord-blood (CB)-derived CD34+ cells exposed to either research- or clinical-grade VSV-g pseudotyped (SIN) LV at a high multiplicity of infection, matching current clinical vector dose requirements. As controls, cells were exposed to poly(I:C), non-transducing Env-less (Bald) or heat inactivated control vectors or kept in culture untreated (FIG. 1A). Key pathways significantly modulated in time by the different treatments were assessed by Term Enrichment Analysis considering various annotated pathway databases (KEGG and WikiPathways) and Gene Ontology Biological Processes (GO-BP), further clustered according to their semantical similarities. The greatest transcriptional variance within our dataset was time in culture, as samples clustered in three distinct temporal groups following Principal Component Analysis (PCA), independently of the treatment group (FIG. 7A). The mere culture of HSPCs in the presence of growth-promoting cytokines resulted in the transcriptional modulation of around 6000-9000 genes over time for all treatment categories (Table 1). For untreated HSPCs, the most enriched pathway was the MAPK signalling (FIG. 7B), in accordance with growth factor and cytokine-induced stimulation (Geest, C. R. et al. (2009) J. Leukoc. Biol. 86:237-250). Poly(I:C)-exposed HSPCs strongly upregulated innate immune responses, significantly mobilising a total of 2691 genes (nominal p-value<0.05) as compared to untreated controls (Table 2). We performed Term Enrichment analysis of the significantly modulated genes to highlight the biological processes affected by Poly(I:C) exposure in HSPCs. Regulation of immune responses, NF-κB signalling, antiviral responses, programmed cell death and antigen processing were among the most represented GO-BP categories (FIG. 8). Instead, LV-exposed HSPCs showed much milder responses, modulating 321 and 281 genes (nominal p-value<0.05) with research- and clinical-grade vectors, respectively (Table 2). The genes altered by LV, whether research- or clinical-grade, converged significantly into DNA-damage responses and in particular the p53 signalling pathway (p-value 6.09×10⁻⁹ for lab-grade LV and 5.1×10⁻³ for purified LV) (FIGS. 1B-C; FIGS. 9A and B). No evidence of significant innate immune activation related to TLR-signalling or activation of NF-κB/Interferon Stimulated Gene (ISG) transcription could be detected, even when comparing LV-exposed HSPCs to untreated cells. A closer look into genes primarily involved in innate immune activation did not show significant changes in their patterns of modulation over time (Table 3). Instead, analysis of the differentially expressed genes with a nominal p-value<0.01 in the LV-exposed HSPCs compared to Bald-exposed controls revealed the upregulation, around 18 hours post-transduction, of a cluster of genes mapping to the KEGG p53 signalling pathway (FIGS. 1D and 1E).

Upregulation of some of the most significantly modulated genes involved in p53 signalling was confirmed by Taqman in HSPCs exposed either to LV or the entry-incompetent Bald LV (FIG. 1F). In particular, p21 and PHLDA3, both direct targets of p53 (Kawase, T. et al. (2009) Cell 136:535-550; Espinosa, J. M. et al. (2001) Molecular Cell 8:57-69), were highly induced by LV, reaching 10-fold higher expression levels compared to controls at 48 h post-transduction. In agreement with the RNA-Seq results using the purified LV (FIG. 1), a clinical-grade LV expressing the human ARSA used in recent trials to treat MLD (Biffi, A. et al. (2013) Science 341:1233158) resulted in comparable levels of p21 induction as with the research-grade LV (FIG. 10A). These results suggest that potential contaminants such as plasmid DNA present in research-grade vector preparations (Merten, O. W. et al. (2011) Human Gene Therapy 22:343-356) are not involved in the observed signalling.

Taken together, these observations indicate that LV transduction of human HSPCs specifically triggers early DNA-damage responses rather than innate immune signalling, despite their capacity to rapidly upregulate these pathways upon poly(I:C) exposure.

### Lv-Induced p53 Signalling Requires Reverse Transcription but is Integration-Independent

To further investigate how LV induces p53 signalling in HSPCs, we chose to track p21 mRNA induction as a marker of p53 activation, as it was one of the most responsive genes in our transcriptome profiling experiments (FIG. 1). Induction of p21 was dose-dependent (FIG. 11A) and was specific to human HSPCs, as CD4+ T cells, Lin-murine HSPCs and different human cell lines of hematopoietic origin did not up-regulate p21 upon LV exposure (FIG. 11B). We then investigated induction of p21 in the different CD34+ subpopulations (FIG. 2A), as distinct DNA damage responses have been shown to occur between more primitive haematopoietic stem cells (HSCs) and committed progenitors (Milyavsky, M. et al. (2010) Cell Stem Cell 7:186-197). Overall, LV induced p21 expression to a similar extent in all CD34+ subpopulations, although the most primitive CD34+CD133+CD38-fraction showed higher levels of induction, correlating with the higher transduction levels reached in this cell fraction (FIGS. 2B and 11C).

The p53 pathway can be triggered by various cellular stress signals such as DNA damage, hypoxia or oncogene activation (Riley, T. et al. (2008) Nature Reviews. Molecular Cell Biology 9:402-412). HIV-1 infection has been shown to induce p53-dependent apoptosis of CD4+ T cells through activation of the DNA-dependent protein kinase (DNA-PK) during viral integration (Cooper, A. et al. (2013) Nature 498:376-379). To address whether activation of p53 was dependent on proviral integration also in the context of transduced HSPCs, we exposed cells to equal doses of either integration-competent or defective LV (IDLV) and measured p21 induction at 48 h post transduction. The two vectors induced p21 to similar extent both in CB as well as in bone marrow (BM)-derived CD34+ HSPCs (FIGS. 2C and 2D) at comparable vector DNA input (FIG. 11D). Of note, triggering was not due to vector stock contaminants, LV particle entry nor exposure of cells to viral cores, as neither the Env-less Bald vector nor the genome-less Empty LV lead to upregulation of p21 (FIGS. 2C and 2D). In agreement with the IDLV-mediated up-regulation, p21 induction still occurred in HSPCs transduced in presence of the integrase inhibitor Raltegravir (FIG. 2E), despite an efficient block in LV integration (FIG. 11E). Integration-independent activation of p53 signalling was confirmed by Western blot, FACS and indirect immunofluorescence (IFI) staining in terms of phosphorylation of p53 at Serine 15, increase in basal p53 levels as well as induction of p21 by both LV and IDLV also in mobilised peripheral blood (mPB)-derived CD34+ HSPCs (FIGS. 2G-H and FIGS. 11G-I). Furthermore, no changes in phosphorylated Histone 2AX (γH2AX) foci were observed, in line with a DNA break-independent induction of p53 (FIG. 11I). Finally, p21 up-regulation was abrogated in the presence of the reverse-transcriptase inhibitor 3TC (FIGS. 2E and 11E), suggesting that lentiviral DNA synthesis is required for p53 signalling to occur in HSPCs.

### P53 Induction Requires Active Nuclear Import of Vector DNA

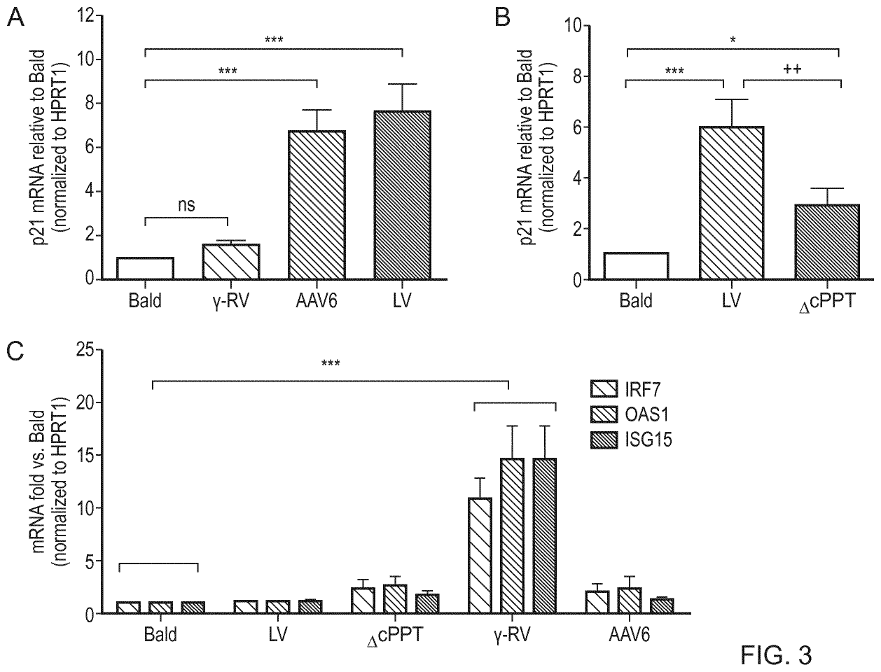

Other viral vectors used to introduce exogenous genetic material into HSPCs (Naldini, L. (2011) Nature Reviews. Genetics 12:301-315) will also expose the cells to exogenous DNA. Similarly to LV, gamma-retroviral vectors (γRV) reverse-transcribe their genomic RNA into DNA that will be integrated into the host genome (Coffin, J. M., Hughes, S. H., and Varmus, H. E. (1997). The Interactions of Retroviruses and their Hosts. In Retroviruses. J. M. Coffin, S. H. Hughes, and H. E. Varmus, editors. Cold Spring Harbor (NY)). Different serotypes of adeno-associated virus vectors (AAV) are instead DNA-based non-integrating viral vectors widely used for transient gene expression or as donor DNA during gene editing (Mingozzi, F. et al. (2011) Nat. Rev. Genet. 12:341-355; Naldini, L. (2015) Nature 526:351-360). We tested the capacity of both γRV and AAV-6 to induce p21 in HSPCs. AAV-6-exposed HSPC showed robust p21 induction, while γRV very moderately affected p21 expression at clinically relevant vector doses (FIGS. 3A and 12A-B). As opposed to HIV-1-derived LV and AAV-6 that actively import the vector DNA into the nucleus of target cells, γRV relies on cellular mitosis to access the host genome (Bushman, F. et al. (2005) Nature Reviews. Microbiology 3:848-858; Nonnenmacher, M. et al. (2012) Gene Therapy 19:649-658). To test if active nuclear import of LV DNA is required to trigger p53 signalling in HSPCs, we generated a vector devoid of the central polypurine tract (cPPT) required for efficient nuclear import of the pre-integration complex (PIC) (Follenzi, A. et al. (2000) Nature Genetics 25:217-222; Zennou, V. et al. (2000) Cell 101:173-185). Up to 3-fold lower nuclear import of the ΔcPPT LV compared to unmodified LV was verified in both 293T cells and CD34+ HSPCs, as measured by lower percentages of nuclear 2LTR circles over total viral DNA 3 days post transduction at comparable input viral cDNA (FIGS. 12C-D). In human HSPCs, the ΔcPPT LV induced p21 mRNA 2-fold less compared to WT LV (FIG. 3B), suggesting that efficient nuclear import of vector DNA is required to activate p53 signalling in HSPCs.

As p53 signalling has recently been linked with type I IFN pathways (Yu, Q. et al. (2015) Cell Reports 11:785-797; Hartlova, A. et al. (2015) Immunity 42:332-343), we also examined expression of several ISG in human HSPCs after exposure to the different vectors. In agreement with our RNA-Seq dataset, no activation of ISG could be evidenced in human HSPCs upon LV transduction, independently of the presence of the cPPT (FIG. 3C). No type I IFN responses were observed in AAV-6 transduced human HSPCs, while γRV triggered significant up-regulation of all ISG tested (FIG. 3C). As opposed to LV-mediated signalling, innate immune activation upon exposure of human HSPC to γRV was independent of reverse-transcription and integration, as ISG induction occurred also in presence of the reverse-transcriptase inhibitor azidothymidine (AZT) or Raltegravir (FIG. 12E). γRV-mediated ISG induction was inhibited when the type I IFN receptor signalling was blocked, but it does not explain lack of p53 signalling upon γRV exposure, as pre-treatment of HSPCs with type I IFN did not prevent induction of p21 by LV (FIG. 11J). Of note, both LV and γRV readily triggered ISG expression in murine Lin-cells (FIG. 12F), indicating that the capacity of LV to avoid type I IFN activation is specific to human HSPCs.

### Functional Consequences of LV-Induced Signalling in HSPCs In Vitro

p53 has a pivotal role in regulating HSC quiescence and homeostasis during both steady-state haematopoiesis as well as under replicative stress (Liu, Y. et al. (2009) Cell Cycle 8:3120-3124; Liu, Y. et al. (2009) Cell Stem Cell 4:37-48; Milyavsky, M. et al. (2010) Cell Stem Cell 7:186-197; Lane, A. A. et al. (2010) Cell 142:360-362; Mohrin, M. et al. (2010) Cell Stem Cell 7:174-185). Depending on the extent of the damage and the transcriptional program activated, cells can undergo several faiths upon p53 activation (Riley, T. et al. (2008) Nature Reviews. Molecular Cell Biology 9:402-412; Brady, C. A. et al. (2011) Cell 145:571-583). Although p21 and PHDLA3 were among the most strongly upregulated genes in our RNA-Seq dataset (FIG. 1), no evident skewing towards a particular p53 transcriptional program could be seen. Therefore, we studied several of the possible functional outcomes of LV-induced p53 signalling in HSPCs. The 10-fold p21 induction observed upon LV transduction (FIG. 13A) led to a slight but significant delay in cellular proliferation rates two days after transduction (FIG. 4A). Similar growth arrest was observed in p21 overexpressing HSPC used as a positive control for this assay (FIG. 4A and FIG. 13A). Slower proliferation was seen in particular in the most primitive CD34+CD133+CD90+ fraction in which this effect seemed to persist up to 6 days post transduction (FIGS. 4A and 13B). In agreement, the portion of cells that had undergone fewer divisions, falling into the population with the highest MFI of cell proliferation dye (C1 group in FIG. 13C), tended to be more represented in the LV-exposed condition as compared to control cells in the bulk CD34+ HSPCs (FIG. 13D). These differences were not due to an increase in the slower proliferating CD34+CD133+CD90+ cells, as no significant difference in the CD34+ subpopulation composition could be observed over time between the different conditions (FIG. 13E). In agreement with slower cell proliferation, transduced HSPCs displayed a higher fraction of cells in the GO cell cycle phase as compared to controls (FIG. 4B). Furthermore, within the transduced population, a stronger proliferation delay was observed for the GFPhigh fraction, likely harbouring more vector copies, compared to the GFPlow ones (FIG. 13D). In agreement, higher p21 induction was detected by FACS in the more transduced BFPhigh cells (FIG. 11G).

LV-exposed HSPCs showed a slight but significant dose-dependent increase in apoptotic cells (FIGS. 4C and 13G). The combined effects of lower proliferation and apoptosis of LV transduced HSPCs was also reflected in terms of total cell counts over time in culture (FIG. 4G). Apoptosis occurred to a similar extent in all CD34+ subpopulations (FIG. 4D) and lower GM-CFU colony output was observed in LV-exposed total HSPCs (FIG. 4E). Looking at the CFU output capacity of the different CD34+× subpopulations, the LV-exposed CD133+CD38− and CD133+CD38int fractions were the ones to show significantly lower GM-CFU counts as compared to controls (FIG. 13F), correlating with higher p53 activation (FIG. 2B). Increased apoptosis also followed exposure of HSPCs to AAV-6 and clinical-grade LV, but does not necessarily correlate with p53 activation as also γRV-exposed HSPC showed similar percentages of apoptotic cells (FIGS. 14A and B). Inhibition of reverse-transcription, but not of integration, completely inhibited LV-mediated induction of apoptosis in HSPCs, correlating with their impact on p21 induction (FIGS. 4F and 2E).

Taken together, these results indicate that the major in vitro functional consequences of p53 triggering upon LV transduction in human HSPCs are a slight delay in proliferation, a higher fraction of cells in the GO phase of the cell cycle as well as increased apoptosis and decrease in clonogenic output.

### Functional Consequences of LV-Induced Signalling in HSPCs In Vivo

Although our in vitro results suggest a functional impact of LV exposure on HSPCs, including on the more primitive CD34+CD133+CD90+ cells, in vivo repopulation studies remain the gold standard to probe HSC self-renewal and differentiation capacity. To determine how LV transduction would affect human HSC function, we transplanted immunocompromised NSG mice with equal and limiting numbers of CB-derived CD34+ HSPCs exposed to either a transducing LV or a genome-less “Empty LV” control vector. To investigate an eventual selective advantage or disadvantage over time of transduced HSPCs, a group of mice received a mix of LV and Empty LV exposed cells in a 3:1 ratio (FIG. 5A). The level of transduction, p53 activation and consequent impact on cell viability and clonogenicity of transplanted HSPCs was verified in vitro (FIGS. 15A-E). Haematopoietic output in peripheral blood, measured as the percentage of human CD45+ cells, was followed over time up to 12 weeks post-transplantation. Despite equal cellular input, LV-exposed HSPCs showed a significantly lower engraftment at all time points compared to cells exposed to the empty vector (FIG. 5B). Decreased engraftment was also confirmed in mPB-derived HSPCs transduced according to the current clinical standard protocol based on two subsequent rounds of transduction with a VSV-g pseudotyped clinical-grade LV (FIGS. 51 and 15G). Of note, no significant differences in the numbers of CD34+ cells retrieved from the bone marrow of NSG mice 16 h after transplantation could be detected between transduced and control HSPCs (FIG. 5J), suggesting that LV transduction does not alter HSPC homing capacity. Nevertheless, once engrafted, no selective disadvantage of transduced HSPCs over controls could be seen. Mice transplanted with the mix of transduced and control HSPCs yielded stable engraftment levels proportional to the original in vitro ratio and taking into account the lower engraftment observed for the LV-exposed cells alone (FIG. 5B). Accordingly, the percentages of transduced GFP+ cells remained constant over time (FIG. 15F). Furthermore, despite reduced engraftment, LV transduction did not alter the lineage composition of human cells in periphery (FIG. 5C).

To assess the impact of LV-induced signalling on long-term repopulating HSCs, we performed secondary transplantations with the CD34+ cells recovered from the three different groups of primary recipients at 12 weeks post-transplantation (FIG. 5A). The percentages of human CD45+ cells retrieved from the bone marrow of the primary recipients at the time of sacrifice reflect the levels observed in the periphery and confirmed significantly lower engraftment of LV-exposed HSPCs (FIG. 5D). However, upon more detailed examination of the bone marrow stem cell compartment, no significant differences in the percentages of total CD34+ cells could be observed between the different groups and equal frequency of more primitive CD34+CD38− and committed CD34+CD38+ cells was seen (FIG. 5E). Within the more primitive CD34+CD38− fraction, the proportion of HSCs, immature lymphoid progenitors (MLP) and multipotent progenitors (MPP) (Doulatov, S. et al. (2012) Cell Stem Cell 10:120-136; Notta, F. et al. (2016) Science 351: aab2116) remained conserved between groups (FIG. 5F), and no differences in lineage composition could be observed in the spleen of sacrificed primary recipients. Upon secondary transplantation, all mice were successfully repopulated independently of the treatment group (FIG. 5G). Although a trend towards lower engraftment of LV-exposed HSPCs was still observed in secondary recipients, the differences compared to the control group were no longer significant. Moreover, no differences were observed between the LV condition and the mice having received a mixed population of transduced and control HSPCs (FIG. 5G). Absence of selective advantage of untransduced control HSPCs was confirmed in this setting as the level of engraftment and percentage of GFP+ cells remained stable over time in the mix condition (FIGS. 5G and 15F). At sacrifice, no major differences in the bone marrow composition were seen in terms of total CD34+ HSPCs and frequency of more primitive CD34+CD38− and committed CD34+CD38+ cells (FIG. 5H). In agreement, a limiting dilution assay (LDA) further confirmed that LV transduction does not significantly alter the long-term repopulating stem cell frequencies (FIG. 5K), although engraftment levels were again slightly lower in the LV-exposed group due to lower numbers of viable cells infused from matched treatment doses (FIG. 15J).

Overall, these results indicate that although exposure to LV negatively impacts HSPC maintenance ex vivo and engraftment in vivo due to acute induction of apoptosis, it does not affect their homing, composition, lineage output or long-term repopulating capacity.

### Inhibition of p53 Activation Rescues HSPC Apoptosis and Engraftment

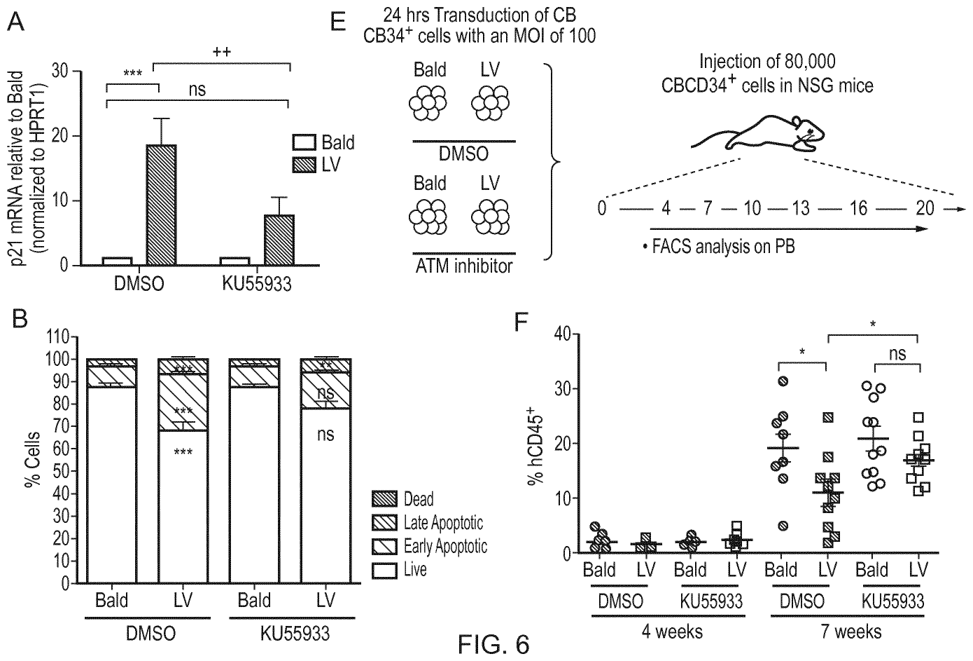

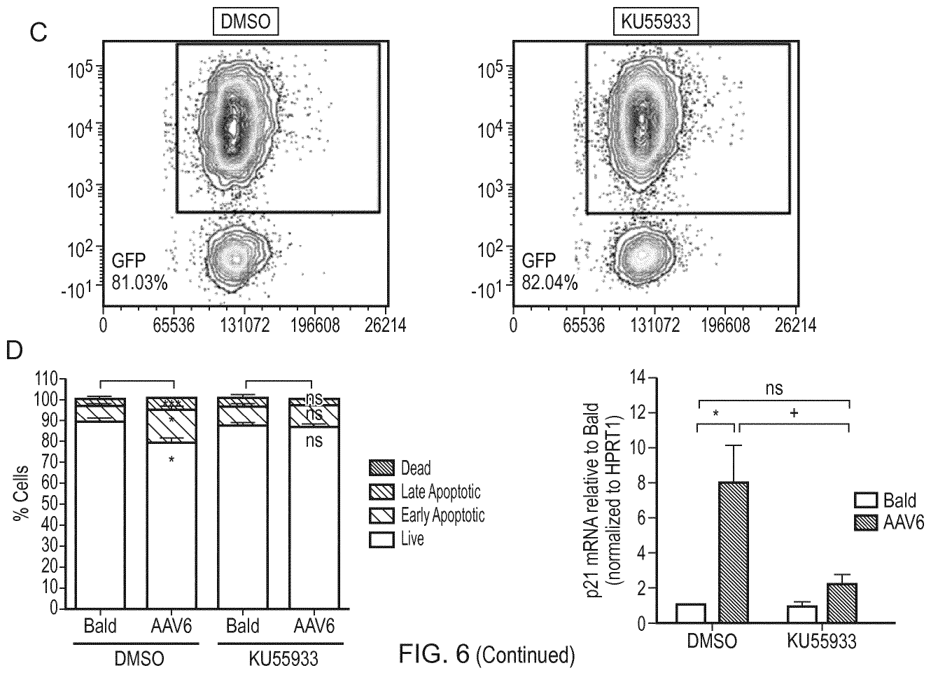

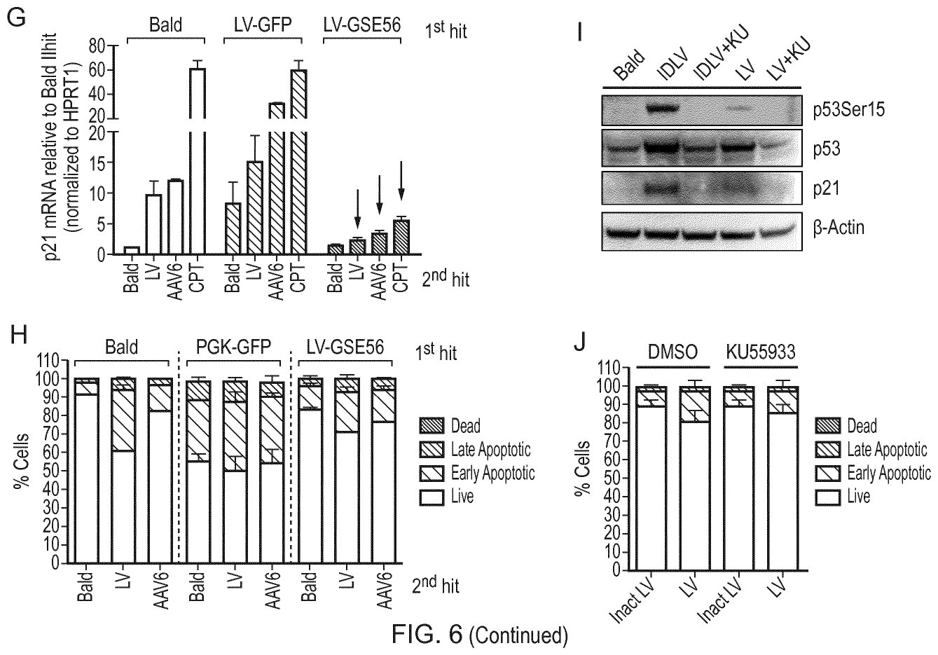

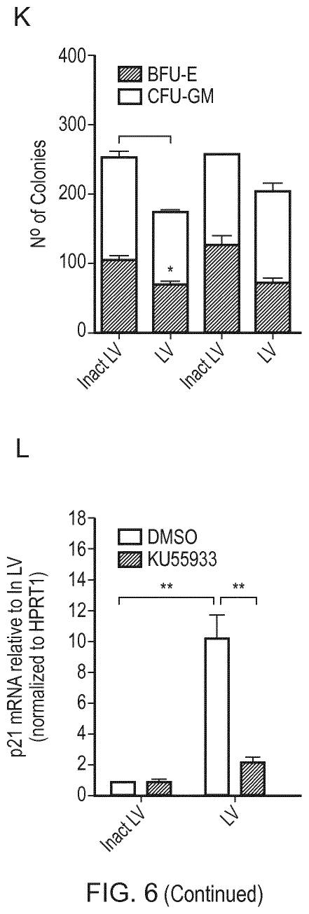

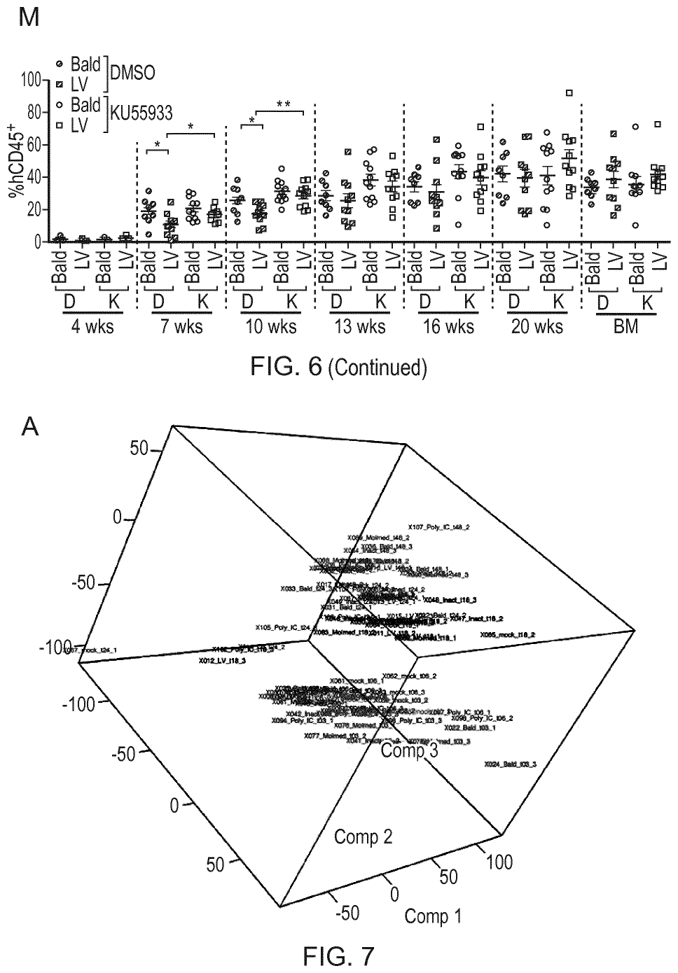

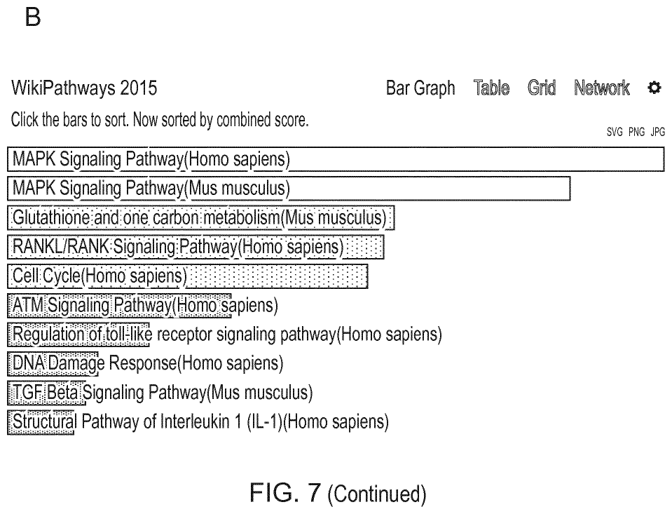

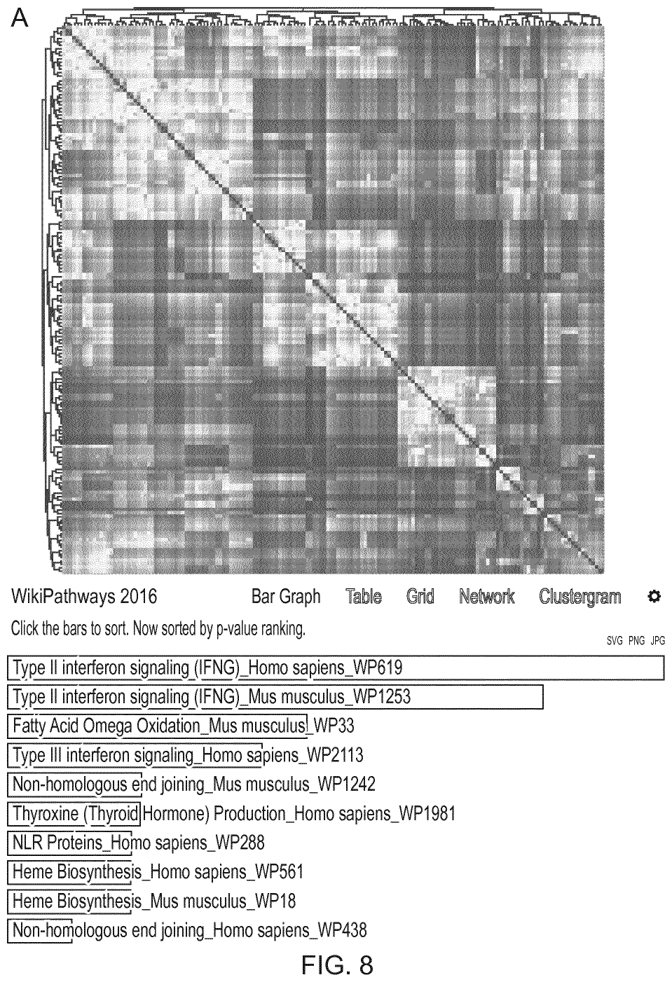

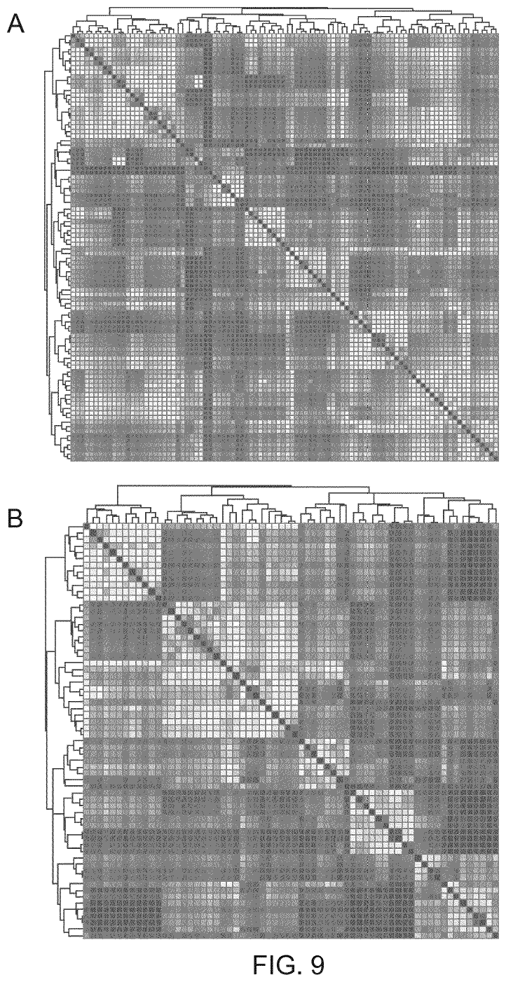

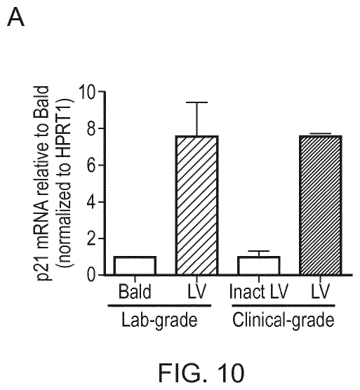

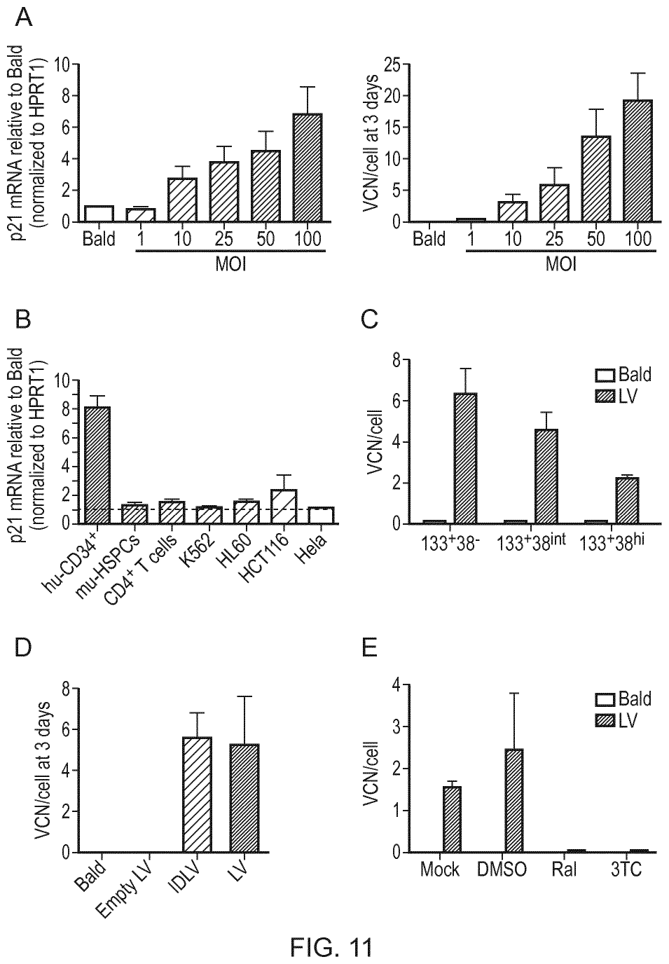

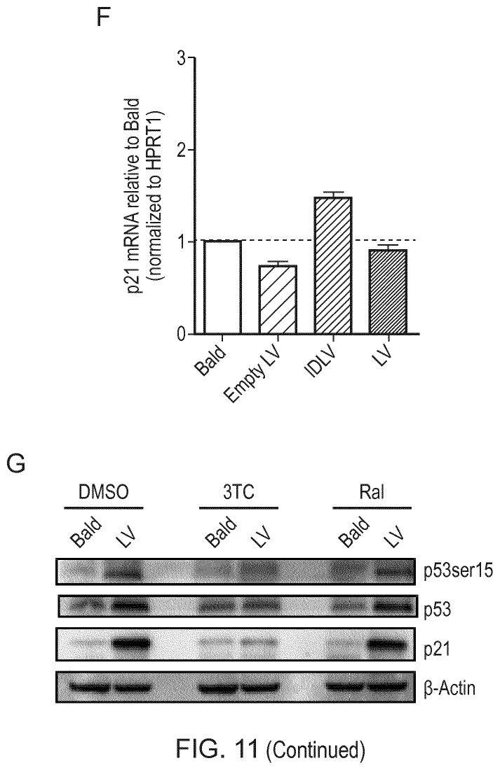

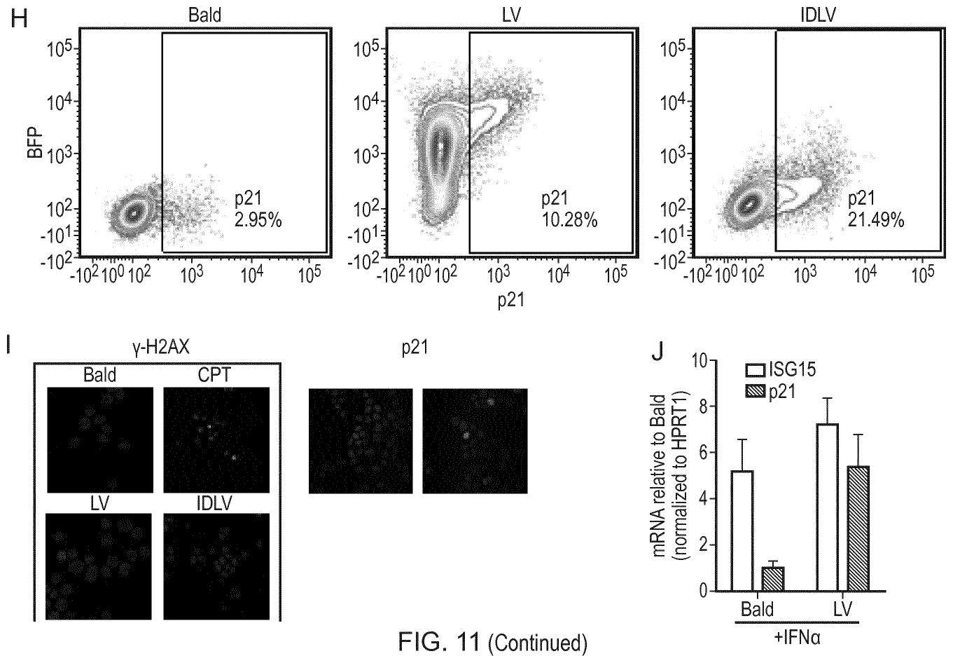

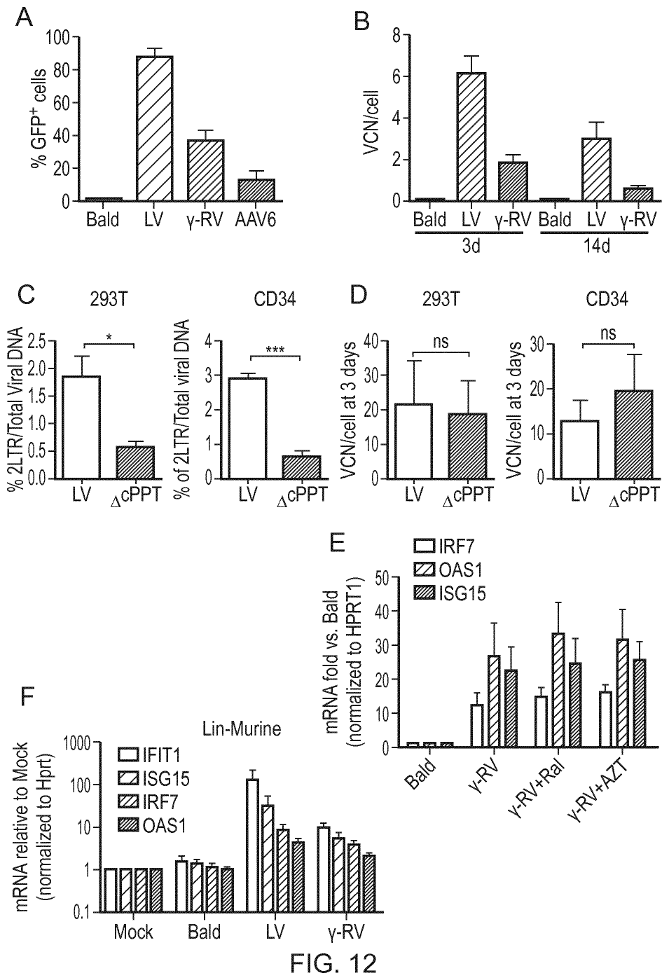

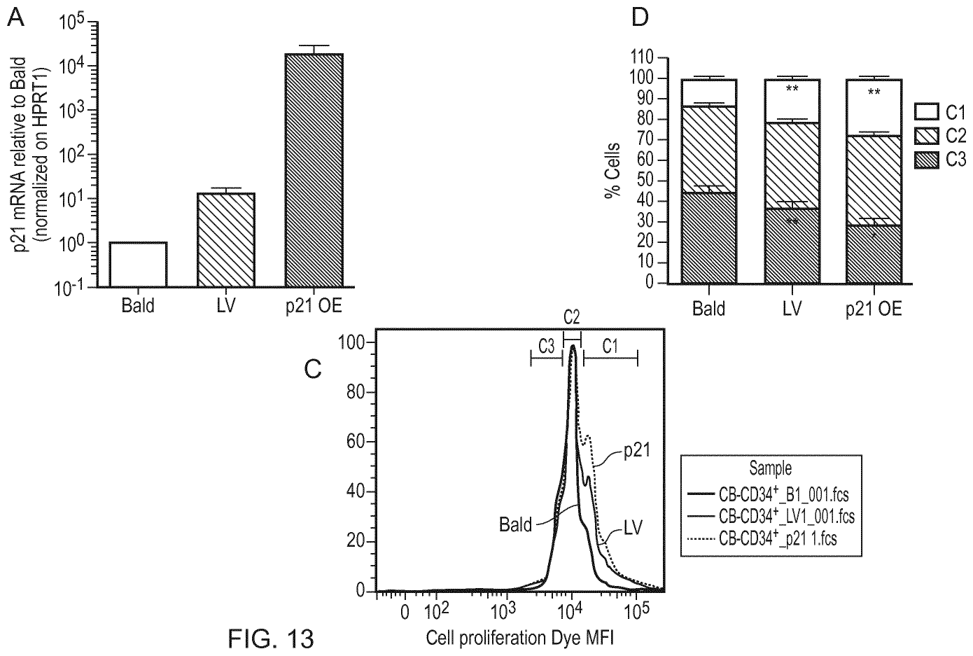

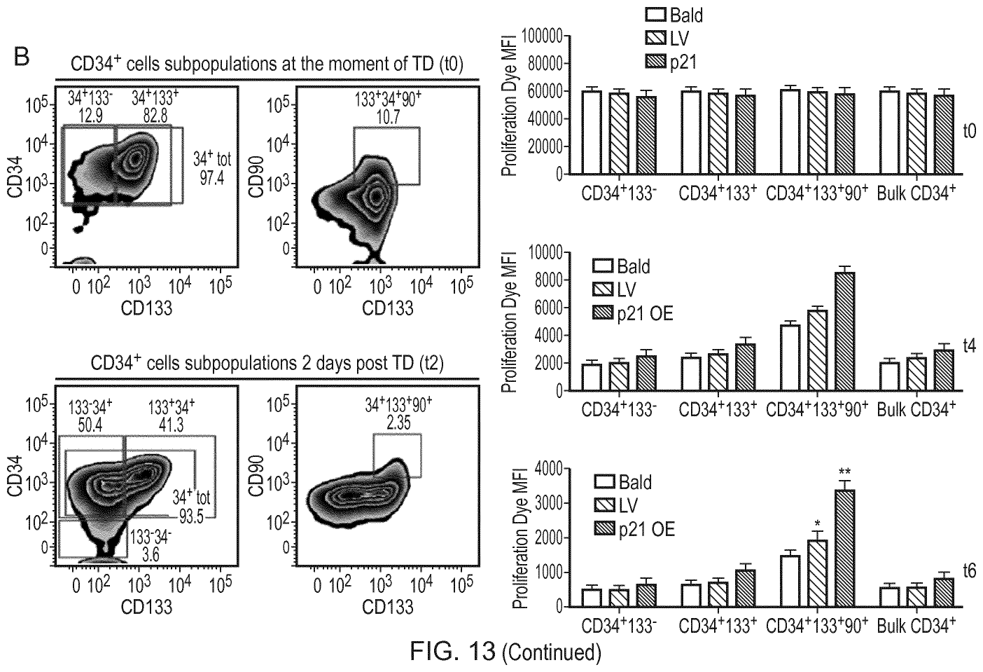

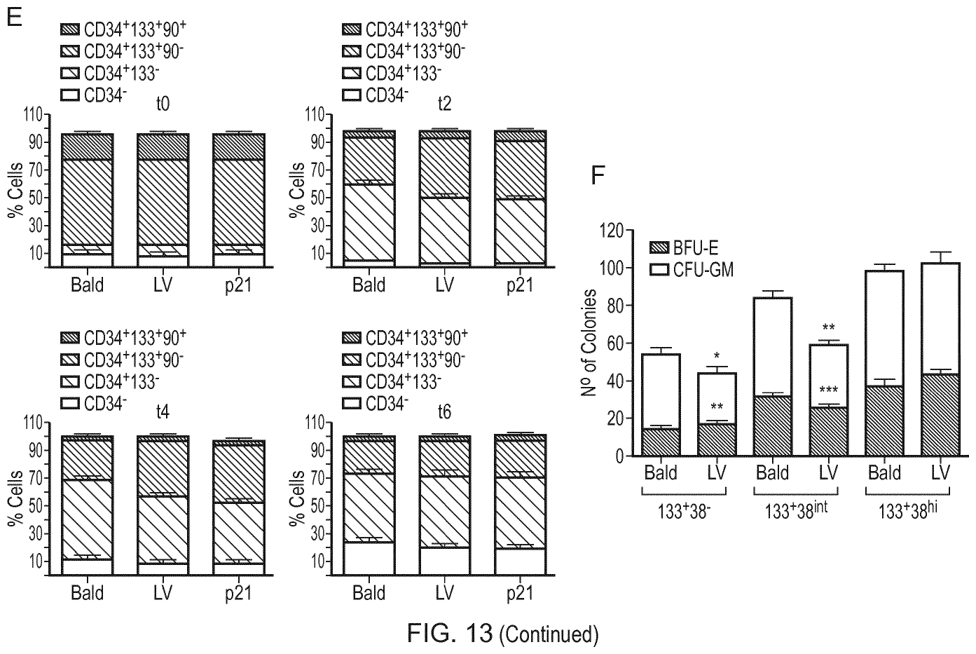

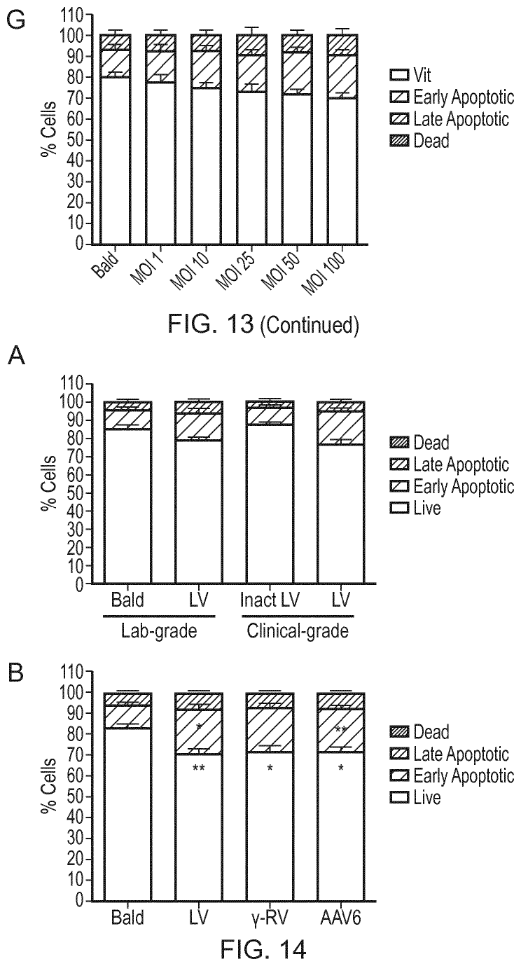

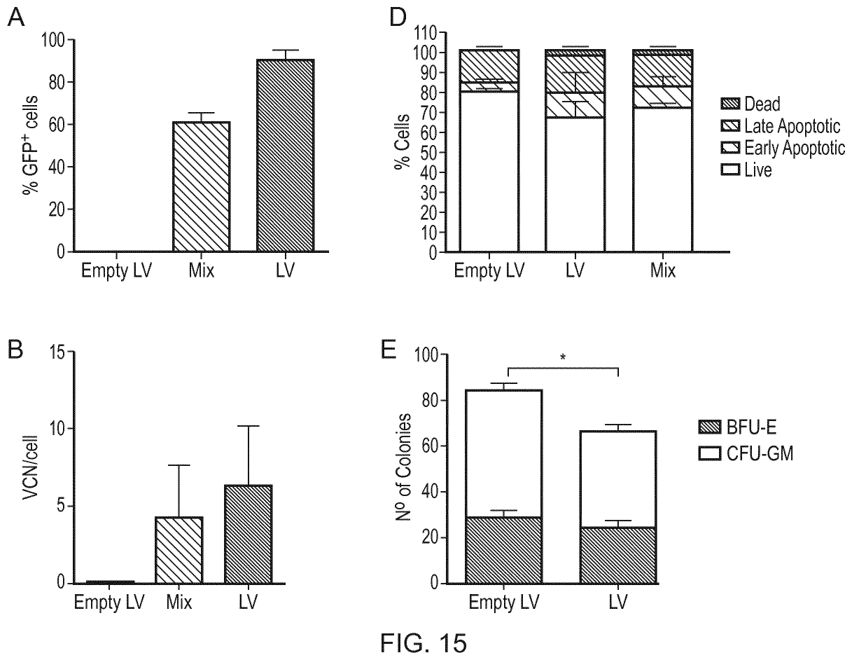

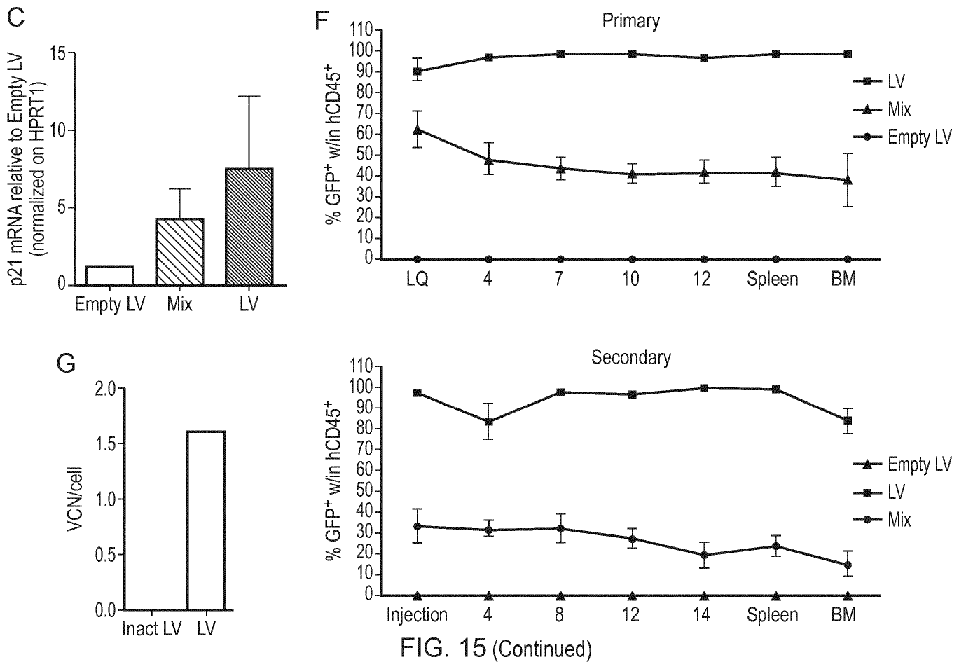

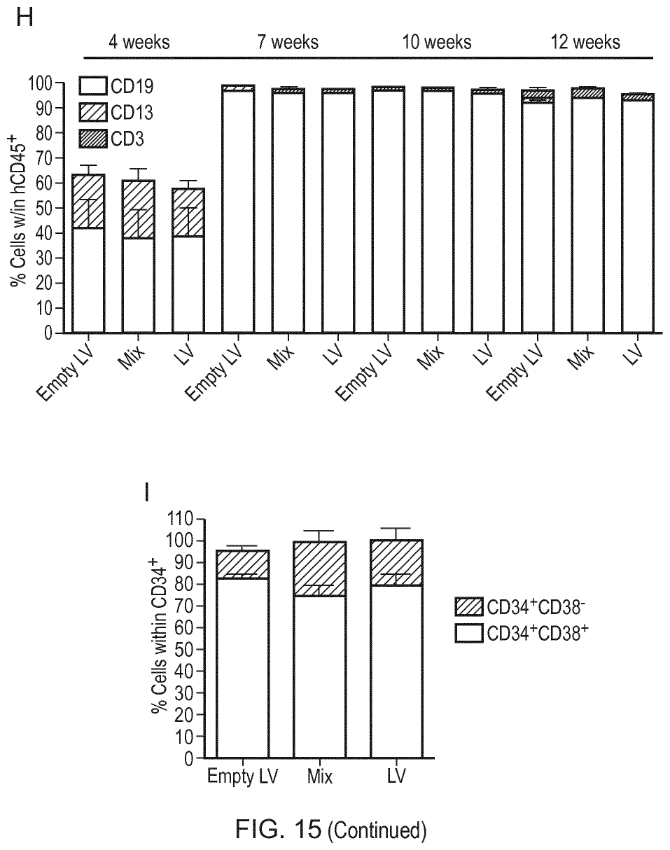

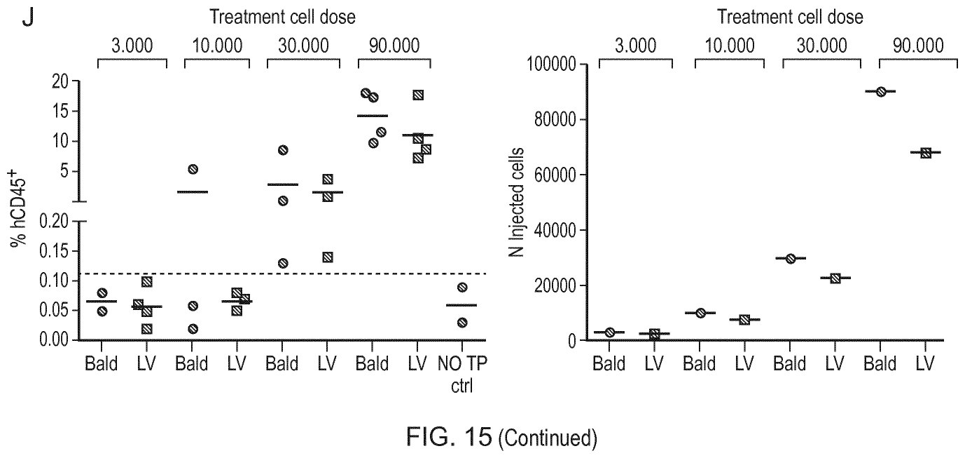

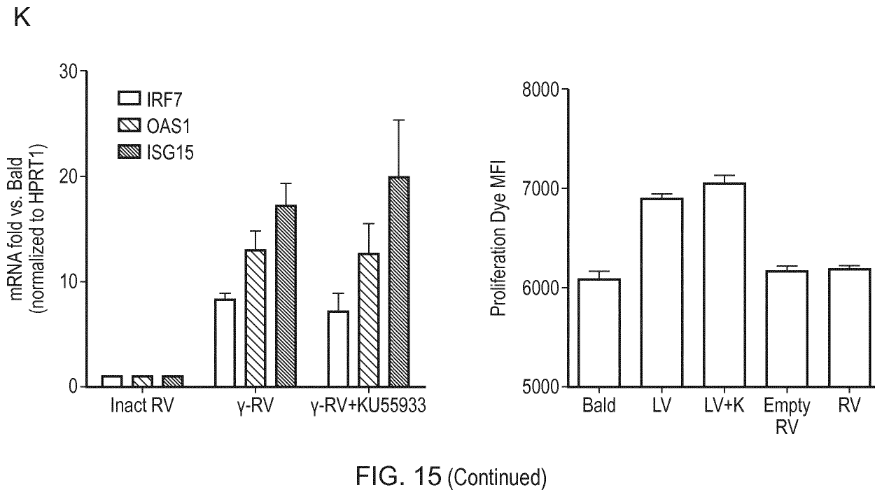

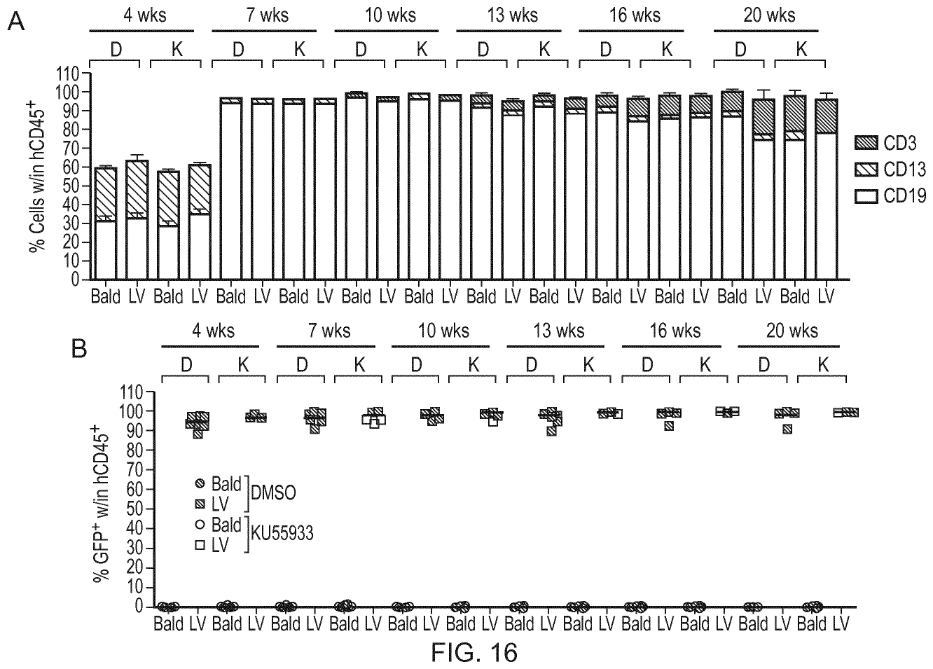

To test whether blocking the p53 signalling during ex vivo HSPC transduction could prevent some of the above described functional consequences, we exposed HSPCs to a control LV or a LV over-expressing a dominant negative form of p53, GSE56 (Milyavsky, M. et al. (2010) Cell Stem Cell 7:186-197; Nucera, S. et al. (2016) Cancer Cell 29:905-921), that we verified to efficiently block p53 signalling in HSPCs even upon strong DNA damage by Captothecin (CPT) (FIG. 6E). Activation of the p53 signalling in terms of p21 induction upon a second round of LV or AAV6 transduction was completely prevented in GSE56-expressing cells compared to control transduced counterparts (FIG. 6E). In agreement, reduced apoptosis was observed in GSE56-expressing HSPCs upon a second round of transduction (FIG. 6F).

Upon cellular stress, p53 signalling can be induced by different upstream signal mediators that will activate it through phosphorylation of certain residues or by inhibiting ubiquitinylation by MDM2 (Riley, T. et al. (2008) Nature Reviews. Molecular Cell Biology 9:402-412). ATM kinase mediated activation leads to phosphorylation of p53 at Serine 15 (Roos, W. P. et al. (2016) Nature Reviews. Cancer 16:20-33), as we observed to occur in HSPCs upon LV exposure (FIG. 3F). To assess involvement of ATM in LV-mediated activation of p53 in human HSPCs, we transduced the cells in the presence of the ATM inhibitor KU55933 (Hickson, I. et al. (2004) Cancer Research 64:9152-9159) and tracked p53 signalling. Inhibition of the ATM kinase during LV exposure significantly reduced the levels of p21 mRNA (FIG. 6A) and partially abrogated induction of apoptosis in HSPCs (FIG. 6B). ATM inhibition did not compromise transduction efficiencies either in terms of GFP+ cells as well as of integrated vector copies (FIG. 6C). Positive impact of ATM inhibition on apoptosis, colony output and cell counts in culture together with reduced p21 induction was confirmed also in the more clinically relevant setting of a two-hit transduction protocol in mPB CD34+ cells using a clinical-grade LV (FIGS. 6H-J). Given that AAV-6-mediated gene transfer triggered similar responses as LV in human HSPCs, we tested whether ATM inhibition could also prevent the in vitro consequences in this setting. Both p21 mRNA induction as well as apoptosis were rescued upon ATM inhibition in CB-CD34+ exposed to AAV-6 (FIG. 6D). ATM inhibition had no impact on γRV-mediated induction of ISG (FIG. 15K). Of note, although ATM inhibition improved cell survival of transduced HSPC it did not affect the observed proliferation delay potentially due to residual p21 activity (FIG. 15K). In agreement with lack of p21 induction, no proliferation delay was detected in γRV transduced HSPCs (FIG. 15K).

To investigate whether transient ATM inhibition during HSPC transduction could rescue their lower engraftment in vivo, cells were exposed to LV or the entry-incompetent Bald vector in presence of KU55933 or the vehicle as control, and transplanted at equal doses into NSG mice (FIG. 6E). Reduced engraftment of LV-exposed HSPCs was confirmed in the control group as compared to Bald-treated cells, both in peripheral blood over time (FIG. 6F). Transient inhibition of p53 signalling during the ex vivo transduction procedure improved LV-exposed HSPC engraftment by around 25% resulting in comparable levels of human CD45+ cells detected in the peripheral blood between transduced and Bald-exposed cells (FIG. 6F). Exposure of HSPCs to the ATM inhibitor did not alter CD34+ nor lineage composition or transduction efficiency in vivo (FIG. 16). Also in this setting long-term human cell engraftment was less affected by LV exposure, further indicating that activation of the p53 signaling upon transduction predominantly impacts short-term HSPC.

Taken together, these results suggest that the in vitro apoptosis and lower in vivo engraftment related to p53 activation in human HSPCs during ex vivo gene transfer can be at least partially prevented by transient inhibition of the upstream mediator ATM without affecting transduction efficiency.

### DISCUSSION

HIV-1 mediated signalling has been widely studied in model cell lines and primary targets of the virus (Towers, G. J. et al. (2014) Cell Host & Microbe 16:10-18) but little information is available in the CD34+ hematopoietic stem cell compartment that remains a debated target for the virus (Josefsson, L. et al. (2012) The Journal of Infectious Diseases 206:28-34; Carter, C. C. et al. (2010) Nature Medicine 16:446-451). The poor permissiveness of these cells to lentiviral transduction in the context of gene therapy approaches has however prompted investigation of the potential immune hurdles and cellular responses to LV as they could hamper efficient and safe gene transfer in HSPCs (Kajaste-Rudnitski, A. et al. (2015) Cellular innate immunity and restriction of viral infection-implications for lentiviral gene therapy in human hematopoietic cells, Human Gene Therapy). Here we have addressed the global transcriptional changes LV transduction triggers early on upon gene transfer in human HSPCs and provide substantial mechanistic insight into the nucleic acid-mediated triggering of DNA damage responses, rather than innate immune signalling, in this primitive stem cell compartment.

Our observation that LV transduction triggers DNA damage responses rather than canonical innate immune activation in human HSPCs underscores their particularity as compared to differentiated hematopoietic cells. Although HIV-1 has been shown to activate p53 signalling in primary CD4+ T cells as well as in the U2OS cell line (Cooper, A. et al. (2013) Nature 498:376-379; Lau, A. et al. (2005) Nat. Cell Biol. 7:493-500), in both cases induction was strictly dependent on viral integration and thus on the generation of physical breaks within the host genome. Furthermore, p21 may not be the preferential downstream effector of p53 activation in this context as we did not observe significant alterations in its expression levels in CD4+ T cells and several other cell lines tested upon exposure to LVs. Our findings suggest that activation of the p53 signalling in human HSPCs upon LV transduction is related instead to nuclear sensing of exogenous DNA, rather than recognition of genomic DNA breaks, as induction occurs independently of integration for LVs and can also be triggered by non-integrating adenoviral DNA.

Nuclear sensing of the double-stranded vector DNA triggered ATM-dependent activation of p53 in human HSPCs. Phosphorylation of the histone variant H2AX is a key feature of ATM-dependent triggering of a cascade of DDR events (Marechal, A. et al. (2013) DNA damage sensing by the ATM and ATR kinases, Cold Spring Harb. Perspect. Biol. 5), but it is not critical for phosphorylation of ATM substrates such as Chk2 and p53 (Kang, J. et al. (2005) Mol. Cell Biol. 25:661-670). In agreement with a break-independent activation of p53 through ATM, we could not detect significant levels of phosphorylated H2AX foci upon transduction. These results also suggest that HIV-1 integration per se may not robustly recruit the DNA repair machinery in human HSPCs, potentially due to steric protection by the viral pre-integration complex (Craigie, R. et al. (2012) Cold Spring Harb Perspect Med 2, a006890). Interestingly, mouse embryonic fibroblasts deficient for the cytoplasmic DNA exonuclease Trex1 show defective G1/S transition and chronic ATM-dependent checkpoint activation, even in the absence of any additional exogenous stress, suggesting that aberrant nucleic acid accumulation, in this case in the ER of Trex1-defective cells, can trigger ATM-dependent DDR responses (Yang, Y. G. et al. (2007) Cell 131:873-886). In the nucleus, ATM has been shown to be activated in the presence of free double-strand (ds) DNA ends in a length-dependent manner (Lee, J. H. et al. (2005) Science 308:551-554). In human cell extracts, ATM is not only activated by blunt dsDNA ends, but also by dsDNA ends with short single-stranded (ss) DNA overhangs (Shiotani, B. et al. (2009) Molecular Cell 33:547-558). This type of molecular pattern, including free 3′ overhangs and secondary structures, often characterise viral and bacterial genetic material and are usually associated with the triggering of innate immune responses through activation of various cytoplasmic nucleic acid sensors (Roers, A. et al. (2016) Immunity 44:739-754).

Both HIV-1 and MLV have been shown to trigger IFN responses through activation of the cytosolic nucleic acid sensor cGAS (Gao, D. et al. (2013) Science 341:903-906). In this context, the retroviral DNA is sensed by cGAS followed by activation of the adaptor protein STING, ultimately leading to synthesis of type I IFN and other pro-inflammatory cytokines. Also the proteins that detect damaged self-DNA in mammalian cells can activate signalling responses that can lead to IFN production, cell cycle regulation and programmed cell death (Jackson, S. P. et al. (2009) Nature 461:1071-1078). Discoveries like ATM-mediated induction of the NF-κB pathways (Wu, Z. H. et al. (2006) Science 311:1141-1146) or DDR-induced IFN signalling in MEF and in the UO2S cell line (Yu, Q. et al. (2015) Cell Reports 11:785-797) highlight the emerging concept of cross-talk between innate immune signalling and DDR. Indeed, poly(I:C)-mediated induction of innate immune responses in HSPCs was also accompanied by significant activation of apoptosis-related pathways. Nevertheless, although we carefully searched for signs of IFN and NF-κB signalling in LV transduced HSPCs, we could not find any significant modulations of these pathways. On the other hand, the MLV-based γRV did trigger substantial expression of several ISG, but this type I IFN activation likely depends on cytosolic recognition of viral RNA, rather than DNA, in human HSPCs. In this setting, the retroviral RNA could be sensed by endosomal TLR, RIG-I or the more recently described zinc-finger antiviral protein (ZAP) (Lee, H. et al. (2013) Proceedings of the National Academy of Sciences of the United States of America 110:12379-12384). Absence of p53 activation is more likely due to the lower transduction efficiencies of YRV in HSPC. The capacity of both LV and AAV to actively enter into the nucleus of non-dividing, quiescent cells (Bushman, F. et al. (2005) Nature Reviews. Microbiology 3:848-858; Nonnenmacher, M. et al. (2012) Gene Therapy 19:649-658), such as the HSPCs, could allow them to evade the cytosolic sensors that instead detect the γRV nucleic acids that potentially accumulate in the cytoplasm while waiting for mitosis to occur. Nevertheless, although less efficient nuclear import upon removal of the cPPT from the LV significantly reduced p21 mRNA induction, it was not accompanied by an induction of ISG. The rates of cytoplasmic accumulation of the ΔcPPT vector may not be sufficient to trigger cytosolic innate sensors, although we cannot exclude that differences in reverse-transcription and/or uncoating rates between LV and γRV or other mechanisms of innate immune evasion exploited by HIV-1 could also avoid IFN responses to LV in human HSPCs (Towers, G. J. et al. (2014) Cell Host & Microbe 16:10-18; Sauter, D. et al. (2016) Curr. Opin. HIV AIDS 11:173-181).

Increased p53 activity has been shown to promote HSC quiescence (Liu, Y. et al. (2009) Cell Stem Cell 4:37-48), but has also been recently associated with loss of HSPCs in the context of a mouse knock-out for type II protein arginine methyltransferase (PRMT5) (Liu, F. et al. (2015) The Journal of Clinical Investigation 125:3532-3544). We observed that in human HSPCs, induction of p53 by LV led to their lower proliferation and a higher fraction of quiescent cells in GO. This, in principle, could preserve the long-term repopulating cells during gene transfer and allow higher engraftment of transduced cells over controls in vivo. The parallel induction of apoptosis seems however to counterbalance these potential benefits, in particular in the fraction containing the short-term repopulating HSPC, as significantly lower percentages of human cells were retrieved from mice having received transduced HSPC. Nevertheless, we did not observe any apparent long-term consequences of the LV-mediated p53 activation in HSPCs, indicating that in the context of ex vivo transduction the transient wave of p53 signalling has only an acute impact on the stem cells.

Although several reports have elegantly addressed the role of DDR and p53 signalling in HSC quiescence and self-renewal, most of these studies have been conducted in the murine context. Quiescent murine HSCs have been shown to resist apoptosis and to repair their DNA by non-homologous end joining (NHEJ), while committed progenitors were more likely to undergo apoptosis or repair their DNA using higher-fidelity homologous recombination (HR) (Mohrin, M. et al. (2010) Cell Stem Cell 7:174-185). In agreement, increased expression of DDR-related genes has been recently shown to characterise more committed murine progenitors over HSCs (Cabezas-Wallscheid, N. et al. (2014) Cell Stem Cell 15:507-522). In contrast to the mouse setting, damaged human HSCs have been shown to preferentially undergo apoptosis after low level irradiation (Milyavsky, M. et al. (2010) Cell Stem Cell 7:186-197). Indeed, we observed that although LV transduction triggered apoptosis in all CD34+ subpopulations, the most affected ones seemed the more primitive CD133+CD38− and CD133+CD38int fractions, also in terms of lower CFU outputs. Nevertheless, we cannot exclude a potential contribution of the higher transduction efficiency observed in the primitive HSC compartment as induction of p53 signalling was vector dose-dependent.

Decreased p53 levels have been shown to rescue human HSCs from irradiation-induced programmed cell death (Milyavsky, M. et al. (2010) Cell Stem Cell 7:186-197). Inhibition of p53 activation during the LV transduction process also partially rescued ex-vivo apoptosis of human HSPCs leading to higher engraftment compared to controls in vivo. Although ATM inhibition has been shown to dramatically decrease LV transduction efficiency in the context of cell lines (Lau, A. et al. (2005) Nat. Cell Biol. 7:493-500), we did not observe any negative impact of KU55933 on LV gene transfer in human HSPCs at similar concentrations. This is most likely due to shorter time of exposure of the cells to the drug in our studies, but cell-type dependent sensitivity to ATM blockade cannot be excluded. Transient inhibition of the p53 signalling cascade could be of clinical benefit for LV-based gene therapy approaches. In particular, LV-mediated signalling could have more pronounced functional consequences in the context of diseases characterised by an elevated pro-inflammatory state or by genetic defects impacting the DDR pathways. However, although we observed only a partial and transient inhibition of p53 induction upon pharmacological ATM blockade in HSPCs, careful evaluation of its potential consequences in terms of LV integrations and genomic stability will be required prior to considering it for clinical testing. Conversely, induction of DDR pathways by IDLV or AAV donor vectors could potentially benefit targeted HSPC gene editing, as one of the major challenges in the field remains the low frequency of HR in the most primitive cell fraction (Naldini, L. (2015) Nature 526:351-360; Genovese, P. et al. (2014) Nature 510:235-240). Development of strategies to specifically block the apoptosis-inducing arm of the vector signalling while preserving the component of DNA damage responses that potentially promote HR could further improve targeted gene editing efficiency in human HSPCs.

Of note, although ATM inhibition rescued LV-induced apoptosis, it did not impact the reduced HSPC proliferation. These data favour the hypothesis that a window of non-apoptotic quiescence can be reached in these conditions yielding improved HSPC engraftment. In this setting, control cells also seemed to benefit from ATM inhibition suggesting that transplantation per se may activate potentially harmful p53 signalling in HSPCs, as also suggested by experiments in which p53 knock-down HSPCs engrafted more compared to control transduced counterparts even in the absence of irradiation-induced DNA damage (Milyavsky, M. et al. (2010) Cell Stem Cell 7:186-197).

The transient wave of p53 signalling did not lead to any apparent long-term consequences as engraftment levels tended to normalise over time between untransduced and treated HSPCs and the long-term repopulating stem cell frequencies remained unaffected, in agreement with unaltered telomere length and gene expression profiles observed in LV-transduced rhesus macaque HSPCs long-term in vivo (Sellers, S. E. et al. (2014) Mol Ther 22:52-58). Our finding that both integrating and non-integrating vectors do not detectably affect the biological properties of long-term HSPCs despite triggering similar molecular responses as observed for the short-term repopulating cells underscores clear biological differences between these two subsets of HSPCs, warranting further investigation in the future. The more persistent proliferation arrest observed in the primitive CD34+CD133+CD90+ fraction could in part account for better preserving the long-term HSC engraftment potential. Furthermore, it is possible that the long-term HSPCs are less sensitive to DDR, as recently reported for quiescent versus activated murine HSPCs (Walter, D. et al. (2015) Nature 520:549-552).

The negative impact LV-mediated p53 signalling has on short-term hematopoietic stem cell (ST-HSC) engraftment is of significant clinical relevance as rapid engraftment of ST-HSC is critical for a safe and successful clinical outcome. Indeed, neutropenia-related infection is reported as the primary cause of death in 8% of autologous hematopoietic stem cell transplantation (HSCT) patients and 17-20% of allogeneic HSCT recipients (Tomblyn, M. et al. (2009) Biol Blood Marrow Transplant 15:1143-1238). Moreover, antibiotic and antifungal prophylaxis can also be associated with significant side effects and does not offer complete protection. Therefore, there is a clear need for alternative strategies to prevent infections in the most vulnerable early neutropenic phase after HSCT (Kandalla, P. K. (2016) J Exp Med 213:2269-2279). Similarly to autologous HSC transplantation, the major cause of treatment related morbidity and mortality in HSC gene therapy can be attributed to prolonged neutropenia due to delayed engraftment. Furthermore, the neutrophil recovery time is longer in the context of gene therapy as compared to normal HSCT, around 4 weeks as compared to 3 weeks for BM-derived HSPC transplantation (Tomblyn, M. et al. (2009) Biol Blood Marrow Transplant 15:1143-1238) and cannot be overcome by increasing the cell dose, as supported also by a recent follow-up report on the metachromatic leukodystrophy (MLD) gene therapy trial (Sessa, M. et al. (2016) Lancet 388:476-487). Clonal tracking studies performed in the context of a gene therapy trial to treat the Wiskott-Aldrich syndrome (WAS) demonstrate the critical role of ST-HSC in these first phases of engraftment and hematopoietic reconstitution in humans (Biasco, L. et al. (2016) Cell Stem Cell 19:107-119). This notion is further consolidated in the murine setting in which the early phase of hematopoietic reconstitution has been shown to be almost exclusively supported by the ST-HSC-enriched CD34+CD38+ fraction of HSPC (Zonari, E. et al. (2017) Stem Cell Reports 8:977-990). Overall, these observations suggest that loss of ST-HSC and the ensuing neutropenia in transplanted patients may be particularly relevant in settings in which ex vivo manipulation of HSCs is required. Although we cannot exclude a potential contribution of the growth conditions applied to HSPCs during their ex vivo manipulation, our results clearly indicate that gene therapy vectors significantly contribute to their delayed engraftment and provide proof-of-principle that dampening the vector signalling can restore hematopoietic reconstitution. Indeed, even a relatively small improvement, such as the 2-fold rescue of ST-HSC engraftment achieved by transiently blocking vector signalling may turn out highly significant from a clinical perspective.

Overall, our studies shed light on the molecular mechanisms and functional consequences of gene therapy vector sensing in human HSPCs. Better knowledge regarding these vector-host interactions will allow the development of more stealth gene therapy protocols tailored for specific disease settings in which vector signalling might impact both gene transfer efficiency as well as HSPC biology. Furthermore, deeper understanding of the signalling cascades activated by non-integrating vector platforms in HSPCs can contribute to the design of more efficient targeted gene editing strategies in the future.

TABLE 1
Number of genes changing over time
under p-value ≤ 0.05.

| | Genes changing |
|---|---|
| Condition | over time |
| Untreated | 5883 |
| Poly(I:C) | 9496 |
| Bald LV | 7307 |
| Lab LV | 7080 |
| Purified LV | 7248 |
| Inactivated purified LV | 7512 |

TABLE 2
Number of differentially expressed genes over time
between the conditions under p-value ≤ 0.05.

| Condition | Genes |
|---|---|
| Untreated Vs. Poly (I:C) | 2691 |
| Untreated Vs. Lab LV | 645 |
| Untreated Vs. Purified LV | 397 |
| Bald LV Vs. Lab LV | 321 |
| Inactivated Purified LV vs. Purified LV | 281 |

TABLE 3
Significance of Selected genes from type I
IFN signalling cascader etrieved from the
comparison between Lab LV and Bald LV

| Gene | p value |
|---|---|
| IFI6 | 0.662955 |
| TVP23A | 0.407102 |
| ISG15 | 0.964351 |
| IFI44L | 0.525873 |
| IFI44 | 0.812837 |
| IFI35 | 0.955392 |
| ISG20 | 0.020014 |
| DDX60 | 0.840026 |
| HERC6 | 0.973159 |
| LGALS3BP | 0.753786 |
| HLA-F | 0.935258 |
| MX1 | 0.904987 |
| PARP12 | 0.924286 |
| EPSTI1 | 0.971483 |
| SAT1 | 0.216319 |
| OAS3 | 0.980706 |
| IFITM1 | 0.861568 |
| HCP5 | 0.831475 |
| IRF7 | 0.80476 |
| XAF1 | 0.796685 |
| DHX58 | 0.764003 |
| H19 | 0.217731 |
| ODF3B | 0.569558 |
| IFI27 | 0.456448 |
| TRIM22 | 0.00061 |
| CSAG3 | 0.369604 |
| USP18 | 0.95428 |
| KLHDC7B | 0.960367 |
| MX2 | 0.948736 |
| OAS1 | 0.983136 |
| HLA-DQA1 | 0.566047 |
| CCL5 | 0.503809 |
| GDF11 | 0.74255 |
| ARG2 | 0.864504 |
| OPTN | 0.953236 |
| OAS2 | 0.971224 |
| GP1BA | 0.714797 |
| USP41 | 0.784604 |
| LAMC2 | 0.256181 |
| STAT1 | 0.586429 |
| PLSCR1 | 0.496799 |
| IFITM3 | 0.441251 |
| HELZ2 | 0.695985 |
| ACO1 | 0.579893 |
| DDX58 | 0.86283 |
| C19orf66 | 0.724447 |
| PARP9 | 0.950643 |
| OASL | 0.832593 |
| CXCL11 | 0.182701 |

TABLE 4
List of anti-human antibodies used for flow cytometry.

| Antibody | Fluorochrome | Dilution | Clone | Company | Code |
|---|---|---|---|---|---|
| hCD235a | APC | 1:25 | GA-R2 | BD Biosciences | 551336 |
| hCD33 | BV421 | 1:25 | WM53 | BD Biosciences | 562854 |
| Anti human FCR | | 1:50 | | Miltenyi Biotec | 120-000-442 |
| Blocking | | | | | |
| Anti murine FCR | | 1:100 | 2.4G2 | BD Pharmigen | 553142 |
| Blocking | | | | | |
| hCD45 | APC-Cy7 | 1:33 | HI30 | eBiosciences | 47-0459-42 |
| hCD19 | PE | 1:25 | SJ25C1 | BD Biosciences | 345789 |
| hCD33 | PeCy7 | 1:25 | P67.6 | BD Biosciences | 333952 |
| hCD3 | APC | 1:25 | UCHT1 | BD Biosciences | 555335 |
| hCD13 | BV | 1:25 | WM15 | BD Biosciences | 562596 |
| hCD34 | PeCy7 | 1:25 | 8G12 | BD Biosciences | 348811 |
| hCD38 | V450 | 1:25 | HB7 | BD Biosciences | 646851 |
| hCD90 | APC | 1:25 | 5E10 | BD Biosciences | 559869 |
| hCD45RA | VioBlue | 1:10 | T6D11 | Miltenyi Biotec | 130-095-464 |
| hCD133 | PE | 1:15 | 293C3 | Miltenyi Biotec | 130-090-853 |
| hCD38 | APC | 1:10 | IB6 | Miltenyi Biotec | 130-092-261 |
| Ki-67 | PE | | B56 | BD Bioesciences | 556027 |
| hCD90 | Brillant Violet | 1:29 | | | |
| Annexin V | Pacific Blue | 1:20 | | Biolegend | 640918 |
| Hoechst | | | | Thermo Fisher | H3570 |
| 7-AAD | | | | Sigma-Aldricht | A9400 |

TABLE 5
List of anti-human antibodies used for WB.

| Antibody | Produced in | Dilution | Incubation | Clone | Company | Code |
|---|---|---|---|---|---|---|
| p53Ser15 | Rabbit | 1:1000 | 5% BSA-TTBS | Polyclonal | Cell signalling Technology | 284S |
| | | | (4° C. O/N) | | | |
| p53 | Mouse | 1:200 | 5% BSA-TTBS | DO-1 | Santa Cruz | Sc126 |
| | | | (4° C. O/N) | | | |
| p21 | Rabbit | 1:1000 | 5% BSA-TTBS | 12D1 | Cell signalling Technology | 2947 |
| | | | (4° C. O/N) | | | |
| γ-H2AX | Rabbit | 1:1000 | 5% BSA-TTBS | 20E3 | Cell signalling Technology | 9718 |
| | | | (4° C. O/N) | | | |
| pSer1981ATM | Rabbit | 1:1000 | 5% BSA-TTBS | D25E5 | Cell signalling Technology | 13050 |
| | | | (4° O/N) | | | |
| ATM | Rabbit | 1:1000 | 5% BSA-TTBS | D2E2 | Cell signalling Technology | 2873 |
| | | | (4° O/N) | | | |

TABLE 6
List of primers used for gene expression.

| Primer | Method | Company | Code |
|---|---|---|---|
| hCDKN1A (p21) | RT-PCR | Thermo Fisher | Hs00355782_m1 |
| hHPRT1 | RT-PCR | Thermo Fisher | Hs01003267_m1 |
| hIRF7 | RT-PCR | Thermo Fisher | Hs01014809_g1 |
| hPHLDA3 | RT-PCR | Thermo Fisher | Hs00385313_m1 |
| hISG15 | RT-PCR | Thermo Fisher | Hs01921425_s1 |
| hOAS1 | RT-PCR | Thermo Fisher | Hs00973637_m1 |
| hDDB2 (p48) | RT-PCR | Thermo Fisher | Hs03044953_m1 |
| hBBC3 (PUMA) | RT-PCR | Thermo Fisher | Hs00248075_m1 |
| mIrf7 | RT-PCR | Thermo Fisher | Mm00516788_m1 |
| mOas1 | RT-PCR | Thermo Fisher | Mm00836412_m1 |
| mIfit1 | RT-PCR | Thermo Fisher | Mm00515153_m1 |
| mIsg15 | RT-PCR | Thermo Fisher | Mm01705338_s1 |
| mHprt | RT-PCR | Thermo Fisher | Mm01545399_m1 |
| mCdkn1a | RT-PCR | Thermo Fisher | Mm04207341_m1 |

TABLE 7
List of primers used for VCN.

| Primer | Sequence | Method | Reference |
|---|---|---|---|
| LV-Sense | 5′-TACTGACGCTCTCGCACC-3′ (SEQ | RT- | (Matrai, J. et al. |
| | ID NO: 9) | PCR/ddPCR | (2011) |
| LV-Antisense | 5′-TCTCGACGCAGGACTCG-3′ (SEQ | RT- | Hepatology |
| | ID NO: 10) | PCR/ddPCR | 53: |
| LV-Probe | FAM-5′- | | 1696- |
| | ATCTCTCTCCTTCCTTCTAGCCTC-3′- | | 1707) |
| | MGBNFQ (SEQ ID NO: 11) | | |
| RT-LV | 5′- | RT- | |
| (ΔU3 sense) | TCACTCCCAACGAAGACAAGATC-3′ | PCR/ddPCR | |
| | (SEQ ID NO: 12) | | |
| RT-LV | 5′-GAGTCCTGCGTCGAGAGAG-3′ | RT- | |
| (Gag antisense) | (SEQ ID NO: 13) | PCR/ddPCR | |
| RT-RV (ΔU3 sense) | 5′-CGAGCTCAATAAAAGAGCCCAC- | ddPCR | |
| | 3′ (SEQ ID NO: 14) | | |
| RT-RV (PBS | 5′-ACAGATAGGTTGCTGGCCAG-3′ | ddPCR | |
| antisense) | (SEQ ID NO: 15) | | |
| hTert fw | 5′-GGCACACGTGGCTTTTCG-3′ (SEQ | RT- | (Lombardo, A. et |
| | ID NO: 16) | PCR/ddPCR | al. (2007) Nature |
| hTert rev | 5′- | RT- | Biotechnology 25: |
| | GGTGAACCTCGTAAGTTTATGCAA- | PCR/ddPCR | 1298-1306) |
| | 3′ (SEQ ID NO: 17) | | |
| hTert Probe | VIC-5′- | | |
| | TCAGGACGTCGAGTGGACACGGTG- | | |
| | 3′-TAMRA (SEQ ID NO: 18) | | |
| 2LTR Fw (2Junct) | 5′- | ddPCR | |
| | CAGTGTGGAAAATCTCTAGCAGTAC- | | |
| | 3′ (SEQ ID NO: 19) | | |
| 2LTR Rev (J2 Rev) | 5′-GCCGTGCGCGCTTCAGCAAGC-3′ | ddPCR | |
| | (SEQ ID NO: 20) | | |

TABLE 8
PCR reactions for dd-PCR.

| | | | | Number | |
|---|---|---|---|---|---|
| | Temperature | | | of | |
| Cycling step | ° C. | Time | | Cycles | |
| Enzyme Activation | 95 | 5 | min | 1 | LV ddPCR |
| Denaturation | 95 | 30 | sec | 40 | |
| Annealing/Extension | 60 | 1 | min | 40 | |
| Signal Stabilisation | 4 | 5 | min | 1 | |
| | 90 | 5 | min | 1 | |
| Hold (Optional) | 4 | Infinite | | 1 | |
| Enzyme Activation | 95 | 5 | min | 1 | RT-LV |
| Denaturation | 95 | 30 | sec | 40 | ddPCR |
| Annealing/Extension | 60-63 | 1 | min | 40 | |
| Signal Stabilisation | 4 | 5 | min | 1 | |
| | 90 | 5 | min | 1 | |
| Hold (Optional) | 4 | Infinite | | 1 | |
| Enzyme Activation | 95 | 5 | min | 1 | RT-RV |
| Denaturation | 95 | 30 | sec | 40 | ddPCR |
| Annealing/Extension | 63 | 1 | min | 40 | |
| Signal Stabilisation | 4 | 5 | min | 1 | |
| | 90 | 5 | min | 1 | |
| Hold (Optional) | 4 | Infinite | | 1 | |
| Enzyme Activation | 95 | 5 | min | 1 | hTERT |
| Denaturation | 95 | 30 | sec | 40 | ddPCR |
| Annealing/Extension | 60-63 | 1 | min | 40 | |
| Signal Stabilisation | 4 | 5 | min | 1 | |
| | 90 | 5 | min | 1 | |
| Hold (Optional) | 4 | Infinite | | 1 | |
| Enzyme Activation | 95 | 5 | min | 1 | 2LTR |
| Denaturation | 95 | 30 | sec | 40 | ddPCR |
| Annealing/Extension | 63 | 1 | min | 40 | |
| Signal Stabilisation | 4 | 5 | min | 1 | |
| | 90 | 5 | min | 1 | |

| | | | |
|---|---|---|---|
| Hold (Optional) | 4 | Infinite | 1 |

TABLE 9
Cell counts of in vivo experiments.

| | N Cell | N Cell | | |
|---|---|---|---|---|
| | Counted | Counted | N Cell | |
| | at TD | at TP | Infused | Source |
| Empty LV | 890000 | 816666 | 80000 | CB |
| LV | 1240000 | 1390000 | 80000 | |
| Empty LV | 840000 | 690000 | 80000 | |
| LV | 1160000 | 900000 | 80000 | |
| Bald DMSO | 700000 | 904000 | 80000 | |
| LV DMSO | 700000 | 840000 | 80000 | |
| Bald KU | 800000 | 957000 | 80000 | |
| LV KU | 800000 | 1030000 | 80000 | |
| Bald DMSO | 625000 | 1000000 | 80000 | |
| LV DMSO | 625000 | 600000 | 80000 | |
| Bald KU | 625000 | 800000 | 80000 | |
| LV KU | 625000 | 600000 | 80000 | |
| Inact LV | 1750000 | 3710000 | 514285 | mPB |
| Clinical-grade LV | 1750000 | 3050000 | 422857 | |
| Bald | 800000 | 1744000 | | CB |
| LV | 800000 | 1216000 | | |
| Bald DMSO | 2000000 | 2200000 | 500000 | CB |
| LV DMSO | 2000000 | 2075000 | 500000 | |

All publications mentioned in the above specification are herein incorporated by reference. Various modifications and variations of the described agents, compositions, uses and methods of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention, which are obvious to those skilled in biochemistry and biotechnology or related fields, are intended to be within the scope of the following claims.

The invention claimed is:

1-26. (canceled)

27. A method of transducing a population of human haematopoietic stem and/or progenitor cells with an integration-defective viral vector, the method comprising:
(a) contacting the population of human haematopoietic stem and/or progenitor cells with an inhibitor of p53 activation during in vitro or ex vivo culture, wherein the inhibition of p53 activation is transient; and
(b) transducing the population of human haematopoietic stem and/or progenitor cells with the integration-defective viral vector during in vitro or ex vivo culture.

28. The method of claim 27, wherein the integration-defective viral vector is an integration-defective lentiviral vector (IDLV) or an adeno-associated viral (AAV) vector.

29. The method of claim 27, wherein the integration-defective viral vector comprises a therapeutic nucleotide of interest.

30. The method of claim 27, wherein the population of human haematopoietic stem and/or progenitor cells is contacted with the inhibitor of p53 activation less than or equal to 48 hours before transducing the population of cells with the integration-defective viral vector.

31. The method of claim 27, wherein the population of human haematopoietic stem and/or progenitor cells is contacted with the inhibitor of p53 activation at the same time as transducing the population of cells with the integration-defective viral vector.

32. The method of claim 27, wherein the population of human haematopoietic stem and/or progenitor cells is contacted with the inhibitor of p53 activation up to 48 hours after transducing the population of cells with the integration-defective viral vector.

33. The method of claim 27, wherein the inhibitor of p53 activation is an ataxia telangiectasia mutated (ATM) kinase inhibitor or a p53 dominant negative peptide.

34. The method of claim 27, wherein the inhibitor of p53 activation is KU-55933 or a derivative thereof; GSE56; KU-60019, CP-466722, Torin 2, CGK 733, or derivatives thereof; or an siRNA, shRNA, miRNA or antisense DNA/RNA.

35. The method of claim 27, wherein the inhibitor of p53 activation is a p53 dominant negative peptide comprising an amino acid sequence having at least 80% identity to SEQ ID NO: 6.

36. The method of claim 27, wherein the inhibitor of p53 activation is KU-55933, GSE56 or KU-60019.

37. The method of claim 27, wherein the population of human haematopoietic stem and/or progenitor cells is obtained from mobilised peripheral blood, bone marrow or umbilical cord blood.

38. The method of claim 27, which includes a further step of enriching the population for haematopoietic stem and/or progenitor cells.

39. A method of gene therapy comprising the steps:
(a) transducing a population of human haematopoietic stem and/or progenitor cells according to the method of claim 27; and
(b) administering the transduced cells to a subject.

40. The method of claim 39, wherein the transduced cells are administered to a subject as part of an autologous stem cell transplant procedure or an allogeneic stem cell transplant procedure.


SEQUENCE LISTING (20 sequences)

SEQ ID NO: 1 - protein, 393 aa, Homo sapiens [sequence omitted]

SEQ ID NO: 2 - protein, 3056 aa, Homo sapiens
MSLVLNDLLICCRQLEHDRATERKKEVEKFKRLIRDPETIKHLDRHSDSKQGKYLNWDAVFRFLQKYIQKETECLRIAKPNVSASTQASRQKKMQEISSLVKYFIKCANRRAPRLKCQELLNYIMDTVKDSSNGAIYGADCSNILLKDILSVRKYWCEISQQQWLELFSVYFRLYLKPSQDVHRVLVARIIHAVTKGCCSQTDGLNSKFLDFFSKAIQCARQEKSSSGLNHILAALTIFLKTLAVNFRIRVCELGDEILPTLLYIWTQHRLNDSLKEVIIELFQLQIYIHHPKGAKTQEKGAYESTKWRSILYNLYDLLVNEISHIGSRGKYSSGFRNIAVKENLIELMADICHQVFNEDTRSLEISQSYTTTQRESSDYSVPCKRKKIELGWEVIKDHLQKSQNDFDLVPWLQIATQLISKYPASLPNCELSPLLMILSQLLPQQRHGERTPYVLRCLTEVALCQDKRSNLESSQKSDLLKLWNKIWCITFRGISSEQIQAENFGLLGAIIQGSLVEVDREFWKLFTGSACRPSCPAVCCLTLALTTSIVPGTVKMGIEQNMCEVNRSFSLKESIMKWLLFYQLEGDLENSTEVPPILHSNFPHLVLEKILVSLTMKNCKAAMNFFQSVPECEHHQKDKEELSFSEVEELFLQTTFDKMDFLTIVRECGIEKHQSSIGFSVHQNLKESLDRCLLGLSEQLLNNYSSEITNSETLVRCSRLLVGVLGCYCYMGVIAEEEAYKSELFQKAKSLMQCAGESITLFKNKTNEEFRIGSLRNMMQLCTRCLSNCTKKSPNKIASGFFLRLLTSKLMNDIADICKSLASFIKKPFDRGEVESMEDDTNGNLMEVEDQSSMNLFNDYPDSSVSDANEPGESQSTIGAINPLAEEYLSKQDLLFLDMLKFLCLCVTTAQTNTVSFRAADIRRKLLMLIDSSTLEPTKSLHLHMYLMLLKELPGEEYPLPMEDVLELLKPLSNVCSLYRRDQDVCKTILNHVLHVVKNLGQSNMDSENTRDAQGQFLTVIGAFWHLTKERKYIFSVRMALVNCLKTLLEADPYSKWAILNVMGKDFPVNEVFTQFLADNHHQVRMLAAESINRLFQDTKGDSSRLLKALPLKLQQTAFENAYLKAQEGMREMSHSAENPETLDEIYNRKSVLLTLIAVVLSCSPICEKQALFALCKSVKENGLEPHLVKKVLEKVSETFGYRRLEDFMASHLDYLVLEWLNLQDTEYNLSSFPFILLNYTNIEDFYRSCYKVLIPHLVIRSHFDEVKSIANQIQEDWKSLLTDCFPKILVNILPYFAYEGTRDSGMAQQRETATKVYDMLKSENLLGKQIDHLFISNLPEIVVELLMTLHEPANSSASQSTDLCDFSGDLDPAPNPPHFPSHVIKATFAYISNCHKTKLKSILEILSKSPDSYQKILLAICEQAAETNNVYKKHRILKIYHLFVSLLLKDIKSGLGGAWAFVLRDVIYTLIHYINQRPSCIMDVSLRSFSLCCDLLSQVCQTAVTYCKDALENHLHVIVGTLIPLVYEQVEVQKQVLDLLKYLVIDNKDNENLYITIKLLDPFPDHVVFKDLRITQQKIKYSRGPFSLLEEINHFLSVSVYDALPLTRLEGLKDLRRQLELHKDQMVDIMRASQDNPQDGIMVKLVVNLLQLSKMAINHTGEKEVLEAVGSCLGEVGPIDFSTIAIQHSKDASYTKALKLFEDKELQWTFIMLTYLNNTLVEDCVKVRSAAVTCLKNILATKTGHSFWEIYKMTTDPMLAYLQPFRTSRKKFLEVPRFDKENPFEGLDDINLWIPLSENHDIWIKTLTCAFLDSGGTKCEILQLLKPMCEVKTDFCQTVLPYLIHDILLQDTNESWRNLLSTHVQGFFTSCLRHFSQTSRSTTPANLDSESEHFFRCCLDKKSQRTMLAVVDYMRRQKRPSSGTIFNDAFWLDLNYLEVAKVAQSCAAHFTALLYAEIYADKKSMDDQEKRSLAFEEGSQSTTISSLSEKSKEETGISLQDLLLEIYRSIGEPDSLYGCGGGKMLQPITRLRTYEHEAMWGKALVTYDLETAIPSSTRQAGIIQALQNLGLCHILSVYLKGLDYENKDWCPELEELHYQAAWRNMQWDHCTSVSKEVEGTSYHESLYNALQSLRDREFSTFYESLKYARVKEVEEMCKRSLESVYSLYPTLSRLQAIGELESIGELFSRSVTHRQLSEVYIKWQKHSQLLKDSDFSFQEPIMALRTVILEILMEKEMDNSQRECIKDILTKHLVELSILARTFKNTQLPERAIFQIKQYNSVSCGVSEWQLEEAQVFWAKKEQSLALSILKQMIKKLDASCAANNPSLKLTYTECLRVCGNWLAETCLENPAVIMQTYLEKAVEVAGNYDGESSDELRNGKMKAFLSLARFSDTQYQRIENYMKSSEFENKQALLKRAKEEVGLLREHKIQTNRYTVKVQRELELDELALRALKEDRKRFLCKAVENYINCLLSGEEHDMWVFRLCSLWLENSGVSEVNGMMKRDGMKIPTYKFLPLMYQLAARMGTKMMGGLGFHEVLNNLISRISMDHPHHTLFIILALANANRDEFLTKPEVARRSRITKNVPKQSSQLDEDRTEAANRIICTIRSRRPQMVRSVEALCDAYIILANLDATQWKTQRKGINIPADQPITKLKNLEDVVVPTMEIKVDHTGEYGNLVTIQSFKAEFRLAGGVNLPKIIDCVGSDGKERRQLVKGRDDLRQDAVMQQVFQMCNTLLQRNTETRKRKLTICTYKVVPLSQRSGVLEWCTGTVPIGEFLVNNEDGAHKRYRPNDFSAFQCQKKMMEVQKKSFEEKYEVFMDVCQNFQPVFRYFCMEKFLDPAIWFEKRLAYTRSVATSSIVGYILGLGDRHVQNILINEQSAELVHIDLGVAFEQGKILPTPETVPFRLTRDIVDGMGITGVEGVFRRCCEKTMEVMRNSQETLLTIVEVLLYDPLFDWTMNPLKALYLQQRPEDETELHPTLNADDQECKRNLSDIDQSFNKVAERVLMRLQEKLKGVEEGTVLSVGGQVNLLIQQAIDPKNLSRLFPGWKAWV

SEQ ID NO: 3 - DNA, 9171 bp, Homo sapiens
  source 1..9171: mol_type = genomic DNA
atgagtctagtacttaatgatctgcttatctgctgccgtcaactagaacatgatagagctacagaacgaaagaaagaagttgagaaatttaagcgcctgattcgagatcctgaaacaattaaacatctagatcggcattcagattccaaacaaggaaaatatttgaattgggatgctgtttttagatttttacagaaatatattcagaaagaaacagaatgtctgagaatagcaaaaccaaatgtatcagcctcaacacaagcctccaggcagaaaaagatgcaggaaatcagtagtttggtcaaatacttcatcaaatgtgcaaacagaagagcacctaggctaaaatgtcaagaactcttaaattatatcatggatacagtgaaagattcatctaatggtgctatttacggagctgattgtagcaacatactactcaaagacattctttctgtgagaaaatactggtgtgaaatatctcagcaacagtggttagaattgttctctgtgtacttcaggctctatctgaaaccttcacaagatgttcatagagttttagtggctagaataattcatgctgttaccaaaggatgctgttctcagactgacggattaaattccaaatttttggactttttttccaaggctattcagtgtgcgagacaagaaaagagctcttcaggtctaaatcatatcttagcagctcttactatcttcctcaagactttggctgtcaactttcgaattcgagtgtgtgaattaggagatgaaattcttcccactttgctttatatttggactcaacataggcttaatgattctttaaaagaagtcattattgaattatttcaactgcaaatttatatccatcatccgaaaggagccaaaacccaagaaaaaggtgcttatgaatcaacaaaatggagaagtattttatacaacttatatgatctgctagtgaatgagataagtcatataggaagtagaggaaagtattcttcaggatttcgtaatattgccgtcaaagaaaatttgattgaattgatggcagatatctgtcaccaggtttttaatgaagataccagatccttggagatttctcaatcttacactactacacaaagagaatctagtgattacagtgtcccttgcaaaaggaagaaaatagaactaggctgggaagtaataaaagatcaccttcagaagtcacagaatgattttgatcttgtgccttggctacagattgcaacccaattaatatcaaagtatcctgcaagtttacctaactgtgagctgtctccattactgatgatactatctcagcttctaccccaacagcgacatggggaacgtacaccatatgtgttacgatgccttacggaagttgcattgtgtcaagacaagaggtcaaacctagaaagctcacaaaagtcagatttattaaaactctggaataaaatttggtgtattacctttcgtggtataagttctgagcaaatacaagctgaaaactttggcttacttggagccataattcagggtagtttagttgaggttgacagagaattctggaagttatttactgggtcagcctgcagaccttcatgtcctgcagtatgctgtttgactttggcactgaccaccagtatagttccaggaacggtaaaaatgggaatagagcaaaatatgtgtgaagtaaatagaagcttttctttaaaggaatcaataatgaaatggctcttattctatcagttagagggtgacttagaaaatagcacagaagtgcctccaattcttcacagtaattttcctcatcttgtactggagaaaattcttgtgagtctcactatgaaaaactgtaaagctgcaatgaattttttccaaagcgtgccagaatgtgaacaccaccaaaaagataaagaagaactttcattctcagaagtagaagaactatttcttcagacaacttttgacaagatggactttttaaccattgtgagagaatgtggtatagaaaagcaccagtccagtattggcttctctgtccaccagaatctcaaggaatcactggatcgctgtcttctgggattatcagaacagcttctgaataattactcatctgagattacaaattcagaaactcttgtccggtgttcacgtcttttggtgggtgtccttggctgctactgttacatgggtgtaatagctgaagaggaagcatataagtcagaattattccagaaagccaagtctctaatgcaatgtgcaggagaaagtatcactctgtttaaaaataagacaaatgaggaattcagaattggttccttgagaaatatgatgcagctatgtacacgttgcttgagcaactgtaccaagaagagtccaaataagattgcatctggctttttcctgcgattgttaacatcaaagctaatgaatgacattgcagatatttgtaaaagtttagcatccttcatcaaaaagccatttgaccgtggagaagtagaatcaatggaagatgatactaatggaaatctaatggaggtggaggatcagtcatccatgaatctatttaacgattaccctgatagtagtgttagtgatgcaaacgaacctggagagagccaaagtaccataggtgccattaatcctttagctgaagaatatctgtcaaagcaagatctacttttcttagacatgctcaagttcttgtgtttgtgtgtaactactgctcagaccaatactgtgtcctttagggcagctgatattcggaggaaattgttaatgttaattgattctagcacgctagaacctaccaaatccctccacctgcatatgtatctaatgcttttaaaggagcttcctggagaagagtaccccttgccaatggaagatgttcttgaacttctgaaaccactatccaatgtgtgttctttgtatcgtcgtgaccaagatgtttgtaaaactattttaaaccatgtccttcatgtagtgaaaaacctaggtcaaagcaatatggactctgagaacacaagggatgctcaaggacagtttcttacagtaattggagcattttggcatctaacaaaggagaggaaatatatattctctgtaagaatggccctagtaaattgccttaaaactttgcttgaggctgatccttattcaaaatgggccattcttaatgtaatgggaaaagactttcctgtaaatgaagtatttacacaatttcttgctgacaatcatcaccaagttcgcatgttggctgcagagtcaatcaatagattgttccaggacacgaagggagattcttccaggttactgaaagcacttcctttgaagcttcagcaaacagcttttgaaaatgcatacttgaaagctcaggaaggaatgagagaaatgtcccatagtgctgagaaccctgaaactttggatgaaatttataatagaaaatctgttttactgacgttgatagctgtggttttatcctgtagccctatctgcgaaaaacaggctttgtttgccctgtgtaaatctgtgaaagagaatggattagaacctcaccttgtgaaaaaggttttagagaaagtttctgaaacttttggatatagacgtttagaagactttatggcatctcatttagattatctggttttggaatggctaaatcttcaagatactgaatacaacttatcttcttttccttttattttattaaactacacaaatattgaggatttctatagatcttgttataaggttttgattccacatctggtgattagaagtcattttgatgaggtgaagtccattgctaatcagattcaagaggactggaaaagtcttctaacagactgctttccaaagattcttgtaaatattcttccttattttgcctatgagggtaccagagacagtgggatggcacagcaaagagagactgctaccaaggtctatgatatgcttaaaagtgaaaacttattgggaaaacagattgatcacttattcattagtaatttaccagagattgtggtggagttattgatgacgttacatgagccagcaaattctagtgccagtcagagcactgacctctgtgacttttcaggggatttggatcctgctcctaatccacctcattttccatcgcatgtgattaaagcaacatttgcctatatcagcaattgtcataaaaccaagttaaaaagcattttagaaattctttccaaaagccctgattcctatcagaaaattcttcttgccatatgtgagcaagcagctgaaacaaataatgtttataagaagcacagaattcttaaaatatatcacctgtttgttagtttattactgaaagatataaaaagtggcttaggaggagcttgggcctttgttcttcgagacgttatttatactttgattcactatatcaaccaaaggccttcttgtatcatggatgtgtcattacgtagcttctccctttgttgtgacttattaagtcaggtttgccagacagccgtgacttactgtaaggatgctctagaaaaccatcttcatgttattgttggtacacttataccccttgtgtatgagcaggtggaggttcagaaacaggtattggacttgttgaaatacttagtgatagataacaaggataatgaaaacctctatatcacgattaagcttttagatccttttcctgaccatgttgtttttaaggatttgcgtattactcagcaaaaaatcaaatacagtagaggacccttttcactcttggaggaaattaaccattttctctcagtaagtgtttatgatgcacttccattgacaagacttgaaggactaaaggatcttcgaagacaactggaactacataaagatcagatggtggacattatgagagcttctcaggataatccgcaagatgggattatggtgaaactagttgtcaatttgttgcagttatccaagatggcaataaaccacactggtgaaaaagaagttctagaggctgttggaagctgcttgggagaagtgggtcctatagatttctctaccatagctatacaacatagtaaagatgcatcttataccaaggcccttaagttatttgaagataaagaacttcagtggaccttcataatgctgacctacctgaataacacactggtagaagattgtgtcaaagttcgatcagcagctgttacctgtttgaaaaacattttagccacaaagactggacatagtttctgggagatttataagatgacaacagatccaatgctggcctatctacagccttttagaacatcaagaaaaaagtttttagaagtacccagatttgacaaagaaaacccttttgaaggcctggatgatataaatctgtggattcctctaagtgaaaatcatgacatttggataaagacactgacttgtgcttttttggacagtggaggcacaaaatgtgaaattcttcaattattaaagccaatgtgtgaagtgaaaactgacttttgtcagactgtacttccatacttgattcatgatattttactccaagatacaaatgaatcatggagaaatctgctttctacacatgttcagggatttttcaccagctgtcttcgacacttctcgcaaacgagccgatccacaacccctgcaaacttggattcagagtcagagcactttttccgatgctgtttggataaaaaatcacaaagaacaatgcttgctgttgtggactacatgagaagacaaaagagaccttcttcaggaacaatttttaatgatgctttctggctggatttaaattatctagaagttgccaaggtagctcagtcttgtgctgctcactttacagctttactctatgcagaaatctatgcagataagaaaagtatggatgatcaagagaaaagaagtcttgcatttgaagaaggaagccagagtacaactatttctagcttgagtgaaaaaagtaaagaagaaactggaataagtttacaggatcttctcttagaaatctacagaagtataggggagccagatagtttgtatggctgtggtggagggaagatgttacaacccattactagactacgaacatatgaacacgaagcaatgtggggcaaagccctagtaacatatgacctcgaaacagcaatcccctcatcaacacgccaggcaggaatcattcaggccttgcagaatttgggactctgccatattctttccgtctatttaaaaggattggattatgaaaataaagactggtgtcctgaactagaagaacttcattaccaagcagcatggaggaatatgcagtgggaccattgcacttccgtcagcaaagaagtagaaggaaccagttaccatgaatcattgtacaatgctctacaatctctaagagacagagaattctctacattttatgaaagtctcaaatatgccagagtaaaagaagtggaagagatgtgtaagcgcagccttgagtctgtgtattcgctctatcccacacttagcaggttgcaggccattggagagctggaaagcattggggagcttttctcaagatcagtcacacatagacaactctctgaagtatatattaagtggcagaaacactcccagcttctcaaggacagtgattttagttttcaggagcctatcatggctctacgcacagtcattttggagatcctgatggaaaaggaaatggacaactcacaaagagaatgtattaaggacattctcaccaaacaccttgtagaactctctatactggccagaactttcaagaacactcagctccctgaaagggcaatatttcaaattaaacagtacaattcagttagctgtggagtctctgagtggcagctggaagaagcacaagtattctgggcaaaaaaggagcagagtcttgccctgagtattctcaagcaaatgatcaagaagttggatgccagctgtgcagcgaacaatcccagcctaaaacttacatacacagaatgtctgagggtttgtggcaactggttagcagaaacgtgcttagaaaatcctgcggtcatcatgcagacctatctagaaaaggcagtagaagttgctggaaattatgatggagaaagtagtgatgagctaagaaatggaaaaatgaaggcatttctctcattagcccggttttcagatactcaataccaaagaattgaaaactacatgaaatcatcggaatttgaaaacaagcaagctctcctgaaaagagccaaagaggaagtaggtctccttagggaacataaaattcagacaaacagatacacagtaaaggttcagcgagagctggagttggatgaattagccctgcgtgcactgaaagaggatcgtaaacgcttcttatgtaaagcagttgaaaattatatcaactgcttattaagtggagaagaacatgatatgtgggtattccgactttgttccctctggcttgaaaattctggagtttctgaagtcaatggcatgatgaagagagacggaatgaagattccaacatataaatttttgcctcttatgtaccaattggctgctagaatggggaccaagatgatgggaggcctaggatttcatgaagtcctcaataatctaatctctagaatttcaatggatcacccccatcacactttgtttattatactggccttagcaaatgcaaacagagatgaatttctgactaaaccagaggtagccagaagaagcagaataactaaaaatgtgcctaaacaaagctctcagcttgatgaggatcgaacagaggctgcaaatagaataatatgtactatcagaagtaggagacctcagatggtcagaagtgttgaggcactttgtgatgcttatattatattagcaaacttagatgccactcagtggaagactcagagaaaaggcataaatattccagcagaccagccaattactaaacttaagaatttagaagatgttgttgtccctactatggaaattaaggtggaccacacaggagaatatggaaatctggtgactatacagtcatttaaagcagaatttcgcttagcaggaggtgtaaatttaccaaaaataatagattgtgtaggttccgatggcaaggagaggagacagcttgttaagggccgtgatgacctgagacaagatgctgtcatgcaacaggtcttccagatgtgtaatacattactgcagagaaacacggaaactaggaagaggaaattaactatctgtacttataaggtggttcccctctctcagcgaagtggtgttcttgaatggtgcacaggaactgtccccattggtgaatttcttgttaacaatgaagatggtgctcataaaagatacaggccaaatgatttcagtgcctttcagtgccaaaagaaaatgatggaggtgcaaaaaaagtcttttgaagagaaatatgaagtcttcatggatgtttgccaaaattttcaaccagttttccgttacttctgcatggaaaaattcttggatccagctatttggtttgagaagcgattggcttatacgcgcagtgtagctacttcttctattgttggttacatacttggacttggtgatagacatgtacagaatatcttgataaatgagcagtcagcagaacttgtacatatagatctaggtgttgcttttgaacagggcaaaatccttcctactcctgagacagttccttttagactcaccagagatattgtggatggcatgggcattacgggtgttgaaggtgtcttcagaagatgctgtgagaaaaccatggaagtgatgagaaactctcaggaaactctgttaaccattgtagaggtccttctatatgatccactctttgactggaccatgaatcctttgaaagctttgtatttacagcagaggccggaagatgaaactgagcttcaccctactctgaatgcagatgaccaagaatgcaaacgaaatctcagtgatattgaccagagtttcaacaaagtagctgaacgtgtcttaatgagactacaagagaaactgaaaggagtggaagaaggcactgtgctcagtgttggtggacaagtgaatttgctcatacagcaggccatagaccccaaaaatctcagccgacttttcccaggatggaaagcttgggtgtga

SEQ ID NO: 4 - protein, 2644 aa, Homo sapiens
MGEHGLELASMIPALRELGSATPEEYNTVVQKPRQILCQFIDRILTDVNVVAVELVKKTDSQPTSVMLLDFIQHIMKSSPLMFVNVSGSHEAKGSCIEFSNWIITRLLRIAATPSCHLLHKKICEVICSLLFLFKSKSPAIFGVLTKELLQLFEDLVYLHRRNVMGHAVEWPVVMSRFLSQLDEHMGYLQSAPLQLMSMQNLEFIEVTLLMVLTRIIAIVFFRRQELLLWQIGCVLLEYGSPKIKSLAISFLTELFQLGGLPAQPASTFFSSFLELLKHLVEMDTDQLKLYEEPLSKLIKTLFPFEAEAYRNIEPVYLNMLLEKLCVMFEDGVLMRLKSDLLKAALCHLLQYFLKFVPAGYESALQVRKVYVRNICKALLDVLGIEVDAEYLLGPLYAALKMESMEIIEEIQCQTQQENLSSNSDGISPKRRRLSSSLNPSKRAPKQTEEIKHVDMNQKSILWSALKQKAESLQISLEYSGLKNPVIEMLEGIAVVLQLTALCTVHCSHQNMNCRTFKDCQHKSKKKPSVVITWMSLDFYTKVLKSCRSLLESVQKLDLEATIDKVVKIYDALIYMQVNSSFEDHILEDLCGMLSLPWIYSHSDDGCLKLTTFAANLLTLSCRISDSYSPQAQSRCVFLLTLFPRRIFLEWRTAVYNWALQSSHEVIRASCVSGFFILLQQQNSCNRVPKILIDKVKDDSDIVKKEFASILGQLVCTLHGMFYLTSSLTEPFSEHGHVDLFCRNLKATSQHECSSSQLKASVCKPFLFLLKKKIPSPVKLAFIDNLHHLCKHLDFREDETDVKAVLGTLLNLMEDPDKDVRVAFSGNIKHILESLDSEDGFIKELFVLRMKEAYTHAQISRNNELKDTLILTTGDIGRAAKGDLVPFALLHLLHCLLSKSASVSGAAYTEIRALVAAKSVKLQSFFSQYKKPICQFLVESLHSSQMTALPNTPCQNADVRKQDVAHQREMALNTLSEIANVFDFPDLNRFLTRTLQVLLPDLAAKASPAASALIRTLGKQLNVNRREILINNFKYIFSHLVCSCSKDELERALHYLKNETEIELGSLLRQDFQGLHNELLLRIGEHYQQVFNGLSILASFASSDDPYQGPRDIISPELMADYLQPKLLGILAFFNMQLLSSSVGIEDKKMALNSLMSLMKLMGPKHVSSVRVKMMTTLRTGLRFKDDFPELCCRAWDCFVRCLDHACLGSLLSHVIVALLPLIHIQPKETAAIFHYLIIENRDAVQDFLHEIYFLPDHPELKKIKAVLQEYRKETSESTDLQTTLQLSMKAIQHENVDVRIHALTSLKETLYKNQEKLIKYATDSETVEPIISQLVTVLLKGCQDANSQARLLCGECLGELGAIDPGRLDFSTTETQGKDFTFVTGVEDSSFAYGLLMELTRAYLAYADNSRAQDSAAYAIQELLSIYDCREMETNGPGHQLWRRFPEHVREILEPHLNTRYKSSQKSTDWSGVKKPIYLSKLGSNFAEWSASWAGYLITKVRHDLASKIFTCCSIMMKHDFKVTIYLLPHILVYVLLGCNQEDQQEVYAEIMAVLKHDDQHTINTQDIASDLCQLSTQTVFSMLDHLTQWARHKFQALKAEKCPHSKSNRNKVDSMVSTVDYEDYQSVTRFLDLIPQDTLAVASFRSKAYTRAVMHFESFITEKKQNIQEHLGFLQKLYAAMHEPDGVAGVSAIRKAEPSLKEQILEHESLGLLRDATACYDRAIQLEPDQIIHYHGVVKSMLGLGQLSTVITQVNGVHANRSEWTDELNTYRVEAAWKLSQWDLVENYLAADGKSTTWSVRLGQLLLSAKKRDITAFYDSLKLVRAEQIVPLSAASFERGSYQRGYEYIVRLHMLCELEHSIKPLFQHSPGDSSQEDSLNWVARLEMTQNSYRAKEPILALRRALLSLNKRPDYNEMVGECWLQSARVARKAGHHQTAYNALLNAGESRLAELYVERAKWLWSKGDVHQALIVLQKGVELCFPENETPPEGKNMLIHGRAMLLVGRFMEETANFESNAIMKKYKDVTACLPEWEDGHFYLAKYYDKLMPMVTDNKMEKQGDLIRYIVLHFGRSLQYGNQFIYQSMPRMLTLWLDYGTKAYEWEKAGRSDRVQMRNDLGKINKVITEHTNYLAPYQFLTAFSQLISRICHSHDEVFVVLMEIIAKVFLAYPQQAMWMMTAVSKSSYPMRVNRCKEILNKAIHMKKSLEKFVGDATRLTDKLLELCNKPVDGSSSTLSMSTHFKMLKKLVEEATFSEILIPLQSVMIPTLPSILGTHANHASHEPFPGHWAYIAGFDDMVEILASLQKPKKISLKGSDGKFYIMMCKPKDDLRKDCRLMEFNSLINKCLRKDAESRRRELHIRTYAVIPLNDECGIIEWVNNTAGLRPILTKLYKEKGVYMTGKELRQCMLPKSAALSEKLKVFREFLLPRHPPIFHEWFLRTFPDPTSWYSSRSAYCRSTAVMSMVGYILGLGDRHGENILFDSLTGECVHVDFNCLFNKGETFEVPEIVPFRLTHNMVNGMGPMGTEGLFRRACEVTMRLMRDQREPLMSVLKTFLHDPLVEWSKPVKGHSKAPLNETGEVVNEKAKTHVLDIEQRLQGVIKTRNRVTGLPLSIEGHVHYLIQEATDENLLCQMYLGWTPYM

SEQ ID NO: 5 - DNA, 7935 bp, Homo sapiens
  source 1..7935: mol_type = genomic DNA
atgggggaacatggcctggagctggcttccatgatccccgccctgcgggagctgggcagtgccacaccagaggaatataatacagttgtacagaagccaagacaaattctgtgtcaattcattgaccggatacttacagatgtaaatgttgttgctgtagaacttgtaaagaaaactgactctcagccaacctccgtgatgttgcttgatttcatccagcatatcatgaaatcctccccacttatgtttgtaaatgtgagtggaagccatgaggccaaaggcagttgtattgaattcagtaattggatcataacgagacttctgcggattgcagcaactccctcctgtcatttgttacacaagaaaatctgtgaagtcatctgttcattattatttctttttaaaagcaagagtcctgctatttttggggtactcacaaaagaattattacaactttttgaagacttggtttacctccatagaagaaatgtgatgggtcatgctgtggaatggccagtggtcatgagccgatttttaagtcaattagatgaacacatgggatatttacaatcagctcctttgcagttgatgagtatgcaaaatttagaatttattgaagtcactttattaatggttcttactcgtattattgcaattgtgttttttagaaggcaagaactcttactttggcagataggttgtgttctgctagagtatggtagtccaaaaattaaatccctagcaattagctttttaacagaactttttcagcttggaggactaccagcacaaccagctagcacttttttcagctcatttttggaattattaaaacaccttgtagaaatggatactgaccaattgaaactctatgaagagccattatcaaagctgataaagacactatttccctttgaagcagaagcttatagaaatattgaacctgtctatttaaatatgctgctggaaaaactctgtgtcatgtttgaagacggtgtgctcatgcggcttaagtctgatttgctaaaagcagctttgtgccatttactgcagtatttccttaaatttgtgccagctgggtatgaatctgctttacaagtcaggaaggtctatgtgagaaatatttgtaaagctcttttggatgtgcttggaattgaggtagatgcagagtacttgttgggcccactttatgcagctttgaaaatggaaagtatggaaatcattgaggagattcaatgccaaactcaacaggaaaacctcagcagtaatagtgatggaatatcacccaaaaggcgtcgtctcagctcgtctctaaacccttctaaaagagcaccaaaacagactgaggaaattaaacatgtggacatgaaccaaaagagcatattatggagtgcactgaaacagaaagctgaatcccttcagatttcccttgaatacagtggcctaaagaatcctgttattgagatgttagaaggaattgctgttgtcttacaactgactgctctgtgtactgttcattgttctcatcaaaacatgaactgccgtactttcaaggactgtcaacataaatccaagaagaaaccttctgtagtgataacttggatgtcattggatttttacacaaaagtgcttaagagctgtagaagtttgttagaatctgttcagaaactggacctggaggcaaccattgataaggtggtgaaaatttatgatgctttgatttatatgcaagtaaacagttcatttgaagatcatatcctggaagatttatgtggtatgctctcacttccatggatttattcccattctgatgatggctgtttaaagttgaccacatttgccgctaatcttctaacattaagctgtaggatttcagatagctattcaccacaggcacaatcacgatgtgtgtttcttctgactctgtttccaagaagaatattccttgagtggagaacagcagtttacaactgggccctgcagagctcccatgaagtaatccgggctagttgtgttagtggattttttatcttattgcagcagcagaattcttgtaacagagttcccaagattcttatagataaagtcaaagatgattctgacattgtcaagaaagaatttgcttctatacttggtcaacttgtctgtactcttcacggcatgttttatctgacaagttctttaacagaacctttctctgaacacggacatgtggacctcttctgtaggaacttgaaagccacttctcaacatgaatgttcatcttctcaactaaaagcttctgtctgcaagccattccttttcctactgaaaaaaaaaatacctagtccagtaaaacttgctttcatagataatctacatcatctttgtaagcatcttgattttagagaagatgaaacagatgtaaaagcagttcttggaactttattaaatttaatggaagatccagacaaagatgttagagtggcttttagtggaaatatcaagcacatattggaatccttggactctgaagatggatttataaaggagctttttgtcttaagaatgaaggaagcatatacacatgcccaaatatcaagaaataatgagctgaaggataccttgattcttacaacaggggatattggaagggccgcaaaaggagatttggtaccatttgcactcttacacttattgcattgtttgttatccaagtcagcatctgtctctggagcagcatacacagaaattagagctctggttgcagctaaaagtgttaaactgcaaagttttttcagccagtataagaaacccatctgtcagtttttggtagaatcccttcactctagtcagatgacagcacttccgaatactccatgccagaatgctgacgtgcgaaaacaagatgtggctcaccagagagaaatggctttaaatacgttgtctgaaattgccaacgttttcgactttcctgatcttaatcgttttcttactaggacattacaagttctactacctgatcttgctgccaaagcaagccctgcagcttctgctctcattcgaactttaggaaaacaattaaatgtcaatcgtagagagattttaataaacaacttcaaatatattttttctcatttggtctgttcttgttccaaagatgaattagaacgtgcccttcattatctgaagaatgaaacagaaattgaactggggagcctgttgagacaagatttccaaggattgcataatgaattattgctgcgtattggagaacactatcaacaggtttttaatggtttgtcaatacttgcctcatttgcatccagtgatgatccatatcagggcccgagagatatcatatcacctgaactgatggctgattatttacaacccaaattgttgggcattttggctttttttaacatgcagttactgagctctagtgttggcattgaagataagaaaatggccttgaacagtttgatgtctttgatgaagttaatgggacccaaacatgtcagttctgtgagggtgaagatgatgaccacactgagaactggccttcgattcaaggatgattttcctgaattgtgttgcagagcttgggactgctttgttcgctgcctggatcatgcttgtctgggctcccttctcagtcatgtaatagtagctttgttacctcttatacacatccagcctaaagaaactgcagctatcttccactacctcataattgaaaacagggatgctgtgcaagattttcttcatgaaatatattttttacctgatcatccagaattaaaaaagataaaagccgttctccaggaatacagaaaggagacctctgagagcactgatcttcagacaactcttcagctctctatgaaggccattcaacatgaaaatgtcgatgttcgtattcatgctcttacaagcttgaaggaaaccttgtataaaaatcaggaaaaactgataaagtatgcaacagacagtgaaacagtagaacctattatctcacagttggtgacagtgcttttgaaaggttgccaagatgcaaactctcaagctcggttgctctgtggggaatgtttaggggaattgggggcgatagatccaggtcgattagatttctcaacaactgaaactcaaggaaaagattttacatttgtgactggagtagaagattcaagctttgcctatggattattgatggagctaacaagagcttaccttgcgtatgctgataatagccgagctcaagattcagctgcctatgccattcaggagttgctttctatttatgactgtagagagatggagaccaacggcccaggtcaccaattgtggaggagatttcctgagcatgttcgggaaatactagaacctcatctaaataccagatacaagagttctcagaagtcaaccgattggtctggagtaaagaagccaatttacttaagtaaattgggtagtaactttgcagaatggtcagcatcttgggcaggttatcttattacaaaggttcgacatgatcttgccagtaaaattttcacctgctgtagcattatgatgaagcatgatttcaaagtgaccatctatcttcttccacatattctggtgtatgtcttactgggttgtaatcaagaagatcagcaggaggtttatgcagaaattatggcagttctaaagcatgacgatcagcataccataaatacccaagacattgcatctgatctgtgtcaactcagtacacagactgtgttctccatgcttgaccatctcacacagtgggcaaggcacaaatttcaggcactgaaagctgagaaatgtccacacagcaaatcaaacagaaataaggtagactcaatggtatctactgtggattatgaagactatcagagtgtaacccgttttctagacctcataccccaggatactctggcagtagcttcctttcgctccaaagcatacacacgagctgtaatgcactttgaatcatttattacagaaaagaagcaaaatattcaggaacatcttggatttttacagaaattgtatgctgctatgcatgaacctgatggagtggccggagtcagtgcaattagaaaggcagaaccatctctaaaagaacagatccttgaacatgaaagccttggcttgctgagggatgccactgcttgttatgacagggctattcagctagaaccagaccagatcattcattatcatggtgtagtaaagtccatgttaggtcttggtcagctgtctactgttatcactcaggtgaatggagtgcatgctaacaggtccgagtggacagatgaattaaacacgtacagagtggaagcagcttggaaattgtcacagtgggatttggtggaaaactatttggcagcagatggaaaatctacaacatggagtgtcagactgggacagctattattatcagccaaaaaaagagatatcacagctttttatgactcactgaaactagtgagagcagaacaaattgtacctctttcagctgcaagctttgaaagaggctcctaccaacgaggatatgaatatattgtgagattgcacatgttatgtgagttggagcatagcatcaaaccacttttccagcattctccaggtgacagttctcaagaagattctctaaactgggtagctcgactagaaatgacccagaattcctacagagccaaggagcctatcctggctctccggagggctttactaagcctcaacaaaagaccagattacaatgaaatggttggagaatgctggctgcagagtgccagggtagctagaaaggctggtcaccaccagacagcctacaatgctctccttaatgcaggggaatcacgactcgctgaactgtacgtggaaagggcaaagtggctctggtccaagggtgatgttcaccaggcactaattgttcttcaaaaaggtgttgaattatgttttcctgaaaatgaaaccccacctgagggtaagaacatgttaatccatggtcgagctatgctactagtgggccgatttatggaagaaacagctaactttgaaagcaatgcaattatgaaaaaatataaggatgtgaccgcgtgcctgccagaatgggaggatgggcatttttaccttgccaagtactatgacaaattgatgcccatggtcacagacaacaaaatggaaaagcaaggtgatctcatccggtatatagttcttcattttggcagatctctacaatatggaaatcagttcatatatcagtcaatgccacgaatgttaactctatggcttgattatggtacaaaggcatatgaatgggaaaaagctggccgctccgatcgtgtacaaatgaggaatgatttgggtaaaataaacaaggttatcacagagcatacaaactatttagctccatatcaatttttgactgctttttcacaattgatctctcgaatttgtcattctcacgatgaagtttttgttgtcttgatggaaataatagccaaagtatttctagcctatcctcaacaagcaatgtggatgatgacagctgtgtcaaagtcatcttatcccatgcgtgtgaacagatgcaaggaaatcctcaataaagctattcatatgaaaaaatccttagagaagtttgttggagatgcaactcgcctaacagataagcttctagaattgtgcaataaaccggttgatggaagtagttccacattaagcatgagcactcattttaaaatgcttaaaaagctggtagaagaagcaacatttagtgaaatcctcattcctctacaatcagtcatgatacctacacttccatcaattctgggtacccatgctaaccatgctagccatgaaccatttcctggacattgggcctatattgcagggtttgatgatatggtggaaattcttgcttctcttcagaaaccaaagaagatttctttaaaaggctcagatggaaagttctacatcatgatgtgtaagccaaaagatgacctgagaaaggattgtagactaatggaattcaattccttgattaataagtgcttaagaaaagatgcagagtctcgtagaagagaacttcatattcgaacatatgcagttattccactaaatgatgaatgtgggattattgaatgggtgaacaacactgctggtttgagacctattctgaccaaactatataaagaaaagggagtgtatatgacaggaaaagaacttcgccagtgtatgctaccaaagtcagcagctttatctgaaaaactcaaagtattccgagaatttctcctgcccaggcatcctcctatttttcatgagtggtttctgagaacattccctgatcctacatcatggtacagtagtagatcagcttactgccgttccactgcagtaatgtcaatggttggttatattctggggcttggagaccgtcatggtgaaaatattctctttgattctttgactggtgaatgcgtacatgtagatttcaattgtcttttcaataagggagaaacctttgaagttccagaaattgtgccatttcgcctgactcataatatggttaatggaatgggtcctatgggaacagagggtctttttcgaagagcatgtgaagttacaatgaggctgatgcgtgatcagcgagagcctttaatgagtgtcttaaagacttttctacatgatcctcttgtggaatggagtaaaccagtgaaagggcattccaaagcgccactgaatgaaactggagaagttgtcaatgaaaaggccaagacccatgttcttgacattgagcagcgactacaaggtgtaatcaagactcgaaatagagtgacaggactgccgttatctattgaaggacatgtgcattaccttatacaggaagctactgatgaaaacttactatgccagatgtatcttggttggactccatatatgtga

SEQ ID NO: 6 - protein, 94 aa, Homo sapiens
CPGRDRRTEEENFRKKEEHCPELPPGSAKRALPTSTSSSPQQKKKPLDGEYFTLKIRGRERFEMFRELNEALELKDARAAEESGDSRAHSSYPK

SEQ ID NO: 7 - DNA, 22 bp, synthetic construct
  misc_feature 1..22: note = Primer
  source 1..22: mol_type = other DNA
cgagctcaataaaagagcccac

SEQ ID NO: 8 - DNA, 20 bp, synthetic construct
  misc_feature 1..20: note = Primer
  source 1..20: mol_type = other DNA
gagtcctgcgtcggagagag

SEQ ID NO: 9 - DNA, 18 bp, synthetic construct
  misc_feature 1..18: note = Primer
  source 1..18: mol_type = other DNA
tactgacgctctcgcacc

SEQ ID NO: 10 - DNA, 17 bp, synthetic construct
  misc_feature 1..17: note = Primer
  source 1..17: mol_type = other DNA
tctcgacgcaggactcg

SEQ ID NO: 11 - DNA, 24 bp, synthetic construct
  misc_feature 1..24: note = Primer
  source 1..24: mol_type = other DNA
atctctctccttccttctagcctc

SEQ ID NO: 12 - DNA, 23 bp, synthetic construct
  misc_feature 1..23: note = Primer
  source 1..23: mol_type = other DNA
tcactcccaacgaagacaagatc

SEQ ID NO: 13 - DNA, 19 bp, synthetic construct
  misc_feature 1..19: note = Primer
  source 1..19: mol_type = other DNA
gagtcctgcgtcgagagag

SEQ ID NO: 14 - DNA, 22 bp, synthetic construct
  misc_feature 1..22: note = Primer
  source 1..22: mol_type = other DNA
cgagctcaataaaagagcccac

SEQ ID NO: 15 - DNA, 20 bp, synthetic construct
  misc_feature 1..20: note = Primer
  source 1..20: mol_type = other DNA
acagataggttgctggccag

SEQ ID NO: 16 - DNA, 18 bp, synthetic construct
  misc_feature 1..18: note = Primer
  source 1..18: mol_type = other DNA
ggcacacgtggcttttcg

SEQ ID NO: 17 - DNA, 24 bp, synthetic construct
  misc_feature 1..24: note = Primer
  source 1..24: mol_type = other DNA
ggtgaacctcgtaagtttatgcaa

SEQ ID NO: 18 - DNA, 24 bp, synthetic construct
  misc_feature 1..24: note = Primer
  source 1..24: mol_type = other DNA
tcaggacgtcgagtggacacggtg

SEQ ID NO: 19 - DNA, 25 bp, synthetic construct
  misc_feature 1..25: note = Primer
  source 1..25: mol_type = other DNA
cagtgtggaaaatctctagcagtac

SEQ ID NO: 20 - DNA, 21 bp, synthetic construct
  misc_feature 1..21: note = Primer
  source 1..21: mol_type = other DNA
gccgtgcgcgcttcagcaagc